(19) United States
(12) Patent Application Publication
(10) Pub. No.: US 2020/0155526 A1
(43) Pub. Date: May 21, 2020

(54) TARGETING LYSINE DEMETHYLASES (KDMS) AS A THERAPEUTIC STRATEGY FOR DIFFUSE LARGE B-CELL LYMPHOMA

(71) Applicant: THE CHILDREN'S MEDICAL CENTER CORPORATION, Boston, MA (US)

(72) Inventors: George Q. DALEY, Cambridge, MA (US); Deepak K. JHA, Boston, MA (US)

(73) Assignee: THE CHILDREN'S MEDICAL CENTER CORPORATION, Boston, MA (US)

(21) Appl. No.: 16/616,782

(22) PCT Filed: May 31, 2018
(86) PCT No.: PCT/US2018/035336

§ 371 Date: Nov. 25, 2019

Related U.S. Application Data
(60) Provisional application No. 62/512,924, filed on May 31, 2017.

(51) Int. Cl. A61K 31/444; C12Q 1/6886; A61P 35/00

(52) U.S. Cl. CPC A61K 31/444; C12Q 1/6886; A61K 31/506; C12Q 2600/106; C12Q 2600/156; A61P 35/00

(57) ABSTRACT

Described herein are methods for treating cancer. Aspects of the invention relate to administering to a subject a compound that targets a KDM4 or KDM5 family member, wherein the subject has at least one mutation in an epigenetic modifier selected from the group consisting of: EZH2, KMT2D, CREBPP, and EP300. In one embodiment, the compound is J1B04. Another aspect of the invention relates to a method of treating diffuse large B-cell lymphoma.

### CROSS-REFERENCE TO RELATED APPLICATIONS

This application claims benefit under 35 U.S.C. § 119(e) of the U.S. Provisional Application Ser. No. 62/512,924, filed May 31, 2017, content of which is incorporated by reference in its entirety.

### FIELD OF THE INVENTION

The field of the invention relates to methods for the treatment of cancer. More specifically, the invention relates to a method of treating cancer by administering to a subject a compound that targets a KDM4 or KDM5 family member.

### BACKGROUND

Despite considerable insights from cancer genome sequencing and advances in targeted chemotherapy and immuno-oncology, cancer remains the second leading killer in the US. Diffuse large B cell lymphoma (DLBCL) is the most common form of Non-Hodgkin Lymphoma (NHL) in the US, accounting for 30% of NHL per year (N. Howlader et al., SEER Cancer Statistics Review, 1975-2012., (2015)). It is the most common type of non-Hodgkin lymphoma among adults, with an annual incidence of 7-8 cases per 100,000 people per year. This cancer occurs primarily in older individuals, with a median age of diagnosis at approximately 70 years of age, though it can also occur in children and young adults in rare cases. DLBCL is an aggressive tumor which can arise in virtually any part of the body, and the first sign of this illness is typically the observation of a rapidly growing mass, sometimes associated with systematic symptoms, e.g., fever, weight loss, and night sweats.

A combination of chemotherapy and the monoclonal antibody rituximab (Rituxan), with or without radiation therapy, is used to treat the majority of patients with DLBCL. The most widely used treatment for DLBCL is R-CHOP (rituximab, cyclophosphamide, doxorubicin, vincristine, and prednisone) that is usually given in 21-day cycles. Sometimes another chemotherapy drug, etoposide (VePesid, Toposar, Etopophos), is added to the R-CHOP regimen, resulting in a drug combination called R-EPOCH. For many patients, the initial treatment is effective and DLBCL does not return after treatment; however, for patients in whom the disease becomes refractory (does not respond to treatment) or relapses (returns after treatment), high-dose chemotherapy coupled with stem cell transplantation can be used to treat patients with DLBCL are used. Relapsedirefractory patients who are not candidates for stem cell transplant, or who choose not to have a stem cell transplant, do have various combination chemotherapy regimens that can sometimes be used for treatment. Bendamustine (Treanda) plus rituximab, single-agent rituximab, lenalidomide (Revlimid) plus rituximab, and gemcitabine-based combinations are secondary therapies that may also be used in these patients, although none of these agents or regimens has been indicated for DLBCL patients.

Using the standard-of-care regimen of Rituximab-Cyclophosphamide-Doxorubicin-Vincristine and Prednisolone (R-CHOP), the current five-year survival rate is approximately 50-60%. For the remaining patient population, R-CHOP either does not work or the patient develops resistance. DLBCL can further be classified on the basis of gene expression profiles into two broad classes—the germinal center B-cells (GCB), and the activated B-cell (ABC) types (Alizadeh, A. A., et al., Nature, 403, 503-511, (2000)). The GCB-DLBCL lymphoma has better prognosis than the ABC type.

Recent cancer genome sequencing studies have revealed that DLBCL patients have a panoply of mutations in several epigenetic modifiers. These mutations are commonly seen in KMT2D, EP300, CREBBP, and EZH2 (Jiang, Y. et al., Seminars hematology, 52, 86-96, (2015); Pasqualucci, L. et. al., Seminars in hematology, 52, 67-76, (2015)). KMT2D is an H3K4 methyltransferase responsible for H3K4me3 while EP300 and CREBBP are histoneacetyltransferases (Black, J. C., et al., Molecular cell, 48, 491-507 (2012)). EZH2 is the major H3K27 methyltransferase (Rothbart, S. B. et al Biochimica et biophysica acta, 1839, 627-643, (2014)). While H3K4me3 and histone acetylation is generally associated with open chromatin, and therefore active transcription, H3K27me3 is associated with heterochromatin formation, and thus regulates gene repression. The mutations that are commonly seen in DLBCL are loss of function (LOF) mutations in KMT2D, EP300, and CREBBP, while they are gain of function (GOF) mutations for EZH2. Therefore, in effect, both these classes (LOF and GOF) of mutations could have the same reinforcing impact on key genes for oncogenesis such as the tumor-suppressor genes. That is, tumor suppressor genes could get suppressed due to high levels of H3K27me3 (arising due to GOF mutations in EZH2), and concomitant reduction of H3K4me3 and, likely, histone acetylation (arising due to LOF of KMT2D, CREBBP and EP300). Consistent with this hypothesis, previous work on KMT2D shows that the LOF of KM2D results in hyper-proliferative phenotype associated with decreased apoptosis and suppression of a number of tumor-suppressor genes. Furthermore, the LOF mutation in CREBBP and EP300 results in mis-regulation of TP53, and BCL6 targets (Pasqualucci, L., et al. Nature, 471, 189-195, (2011); Pasqualucci, L., et al., Nature genetics, 43, 830-837, (2011)). Contrarily, EZH2 GOF mutations repressed cell cycle checkpoints, and key regulatory loci for germinal cell differentiation (Beguelin, W., et al., Cancer cell, 23, 67-692 (2013)). In addition, and interestingly, EZH2 GOF mutations are restricted to the GCB type of DLBCL, while CREBBP/EP300, and KMT2D mutations are found in both GCB and ABC type of lymphoma.

The high mortality rates and ineffective treatments for DLBCL underscoring the need to develop new therapeutic targets, and identify vulnerabilities that can be exploited based on current knowledge. While the inhibition of general transcription using small molecules such as jumonji inhibitors as an approach for cancer treatment shows promise, the application to DLBCL remains an unmet need. The present invention addresses some of these needs.

### SUMMARY

The methods disclosed herein are based, in part, on the discovery that cancer cells having a mutation in at least one of the epigenetic modifiers EZH2, KMT2D, CREBPP, and/or EP300, a mutation in the canonical Wnt-signaling pathway, over-expression of a Ikaros family member, or overexpression of KDM4A and/or KDM4B are more susceptible to treatments that target a KDM4 or KDM5 family member. Accordingly, in one aspect, disclosed herein is a method of treating cancer. Generally, the method comprises administering a therapeutically effective amount of a compound that targets a KDM4 or a KDM5 family member. Further, the subject selected for treatment generally has one or more of the following indications: (i) at least one mutation in an epigenetic modifier selected from the group consisting of EZH2, KMT2D, CREBPP, and EP300; (ii) over-expression of at least one Ikaros family member; (iii) over-expression of KDM4A and/or KDM4C; and/or (iv) at least one mutation in canonical Wnt signaling.

In another aspect, disclosed herein is a method of treating cancer, the method comprising administering an effective amount of JIB04 to a subject in need thereof, wherein: (i) the subject has at least one mutation in an epigenetic modifier selected from the group consisting of EZH2, KMT2D, CREBPP, and EP300; (ii) the subject has over-expression of at least one Ikaros family member; (iii) the subject has over-expression of KDM4A and/or KDM4C; and/or (iv) the subject has at least one mutation in canonical Wnt signaling.

In yet another aspect, disclosed herein is a method for treating DLBCL. Generally, the method comprises diagnosing and/or selecting a subject as having diffuse large B-cell lymphoma (DLBCL) and administering a therapeutically effective amount of a compound targeting a KDM4/KDM5 family member to the subject.

In some embodiments of the various aspects disclosed herein, the method further comprises administering to the subject a therapeutically effective amount of a cyclin-dependent kinase 7 (CDK7) inhibitor or a Burton's tyrosine kinase (BTK) inhibitor.

In some embodiments of the various aspects disclosed herein, the method can include a step of selecting a subject, wherein: (i) the subject has at least one mutation in an epigenetic modifier selected from the group consisting of EZH2, KMT2D, CREBPP, and EP300; (ii) the subject has over-expression of at least one Ikaros family member; (iii) the subject has over-expression of KDM4A and/or KDM4C; and/or (iv) the subject has at least one mutation in canonical Wnt signaling.

Without limitations, selecting a subject can include detecting or assaying for one or more of the following: (i) a mutation in an epigenetic modifier selected from the group consisting of EZH2, KMT2D, CREBPP, and EP300; (ii) over-expression of at least one Ikaros family member; (iii) over-expression of KDM4A and/or KDM4C; and/or (iv) a mutation in canonical Wnt signaling. For example, selecting a subject can include assaying a sample from the subject for presence of a mutation in EZH2, KMT2D, CREBPP, and/or EP300. In another example, selecting a subject can include assaying a sample from the subject for over-expression of a Ikaros family member. In still another example, selecting a subject can include assaying a sample from the subject for over-expression of KDM4A and/or KDM4C. In yet another example, selecting a subject can include assaying a sample from the subject for a mutation in canonical Wnt signaling.

Without limitations, the compound targeting the KDM4 or KDM5 family member can be an inhibitor or activator of said family member. For example, the compound can be an inhibitor of a KDM4 or KDM5 family member.

In embodiments of the various aspects described herein, the compound is an inhibitor of a KDM4 family member. For example, the compound can be a KDM4A, KDM4B, KDM4C or KDM4D inhibitor. In some embodiments, the compound is a KDM4A and/or KDM4C inhibitor.

The compound targeting the KDM4 or KDM5 family member can be an inhibitor of a KDM5 family member. For example, the compound can be a KDM5A or KDM5B inhibitor.

In some embodiments of the various aspects disclosed herein, the compound inhibits KDM4A, KDM4C and/or KDM5A.

In some embodiments, the compound is 5-Chloro-2-[(E)-2-[phenyl(pyridin-2-yl)methylidene]hydrazin-1-yl]pyridine (JIB04).

Nucleic acids that can bind with and reduce the expression of a target nucleic acid are well known in the art. Thus, in some embodiments, the compound targeting the KDM4/KDM5 family member can be a nucleic acid which binds to and reduces or inhibits the expression of a nucleic acid encoding a KDM4/KDM5 family member. Exemplary nucleic acids that can bind with and reduce the expression of a target nucleic acid include, but are not limited to, siRNAs, shRNAs, and antisense oligonucleotides.

In some embodiments of the various aspects disclosed herein, the method further comprises administering an additional anti-cancer therapy to said subject. For example, administering a standard of care chemotherapeutic to said subject.

In some other embodiments of the various aspects disclosed herein, the compound targeting KDM4 or KDM5 family member is administered as a monotherapy. In other words, the method does not include administering an additional anti-cancer therapy to the subject and only the compound targeting KDM4 or KDM5 family member, as is or as comprised in a pharmaceutical composition, is administered.

### BRIEF DESCRIPTION OF THE DRAWINGS

FIG. 1 is a bar graph showing frequent epigenetic alterations in DLBCL.

FIG. 2 is a line graph showing the dose response of cells to two days of treatment with indicated JIB04 concentrations. The response of five DLBCL type cells; Farage, SUDHL5, DB, OCI-Ly1 and Toledo; and four non-DLBCL type cells MV4-11, HL-60, THP-1 and Jurkat, are shown.

FIG. 3 is a block graph of gene mutations for DLBCL and non-DLBCL cell lines.

FIG. 4A shows plotted data from an apoptosis assay using Annexin V staining.

FIG. 4B shows increased production of PARP.

FIG. 5 is a line graph showing the dose response of OCI-Ly1 cells to various Jumonji-inhibitors.

FIG. 6 is a proliferation curve showing the effect of shRNA knockdown.

FIGS. 7A-7C depicts the results of transcriptomics experiments and shows induction of programs that prevent cell proliferation, induce cell death, and, differentiation. FIG. 7A shows GSEA analysis of RNA_seq data revealing key pathways that get altered (up or down).

FIG. 7B is a volcano plot of differentially expressed genes. Includes the key cell cycle regulator CDKN1A. FIG. 7C B shows CDKN1A induced cell cycle arrest at different dosage (top panel) and different times (bottom panel).

FIGS. 8A-8C show that KDM4 inhibition induces the expression of extra-lineage transcription factors. FIG. 8A shows upregulation of transcription factors that contribute to other cell lineages. FIG. 8B shows downregulation of key B-cell genes including the target of Rituximab, anti-CD20. FIG. 8C shows upregulation of genes that regulate Monocyte fate of blood cells.

FIG. 8 is a schematic representation showing KDM4A and KDM4C regulate B-cell identity through Ikaros function.

FIG. 10 shows the CTG signal relative to DMSO on Day 2 of JIB04 and Day 5 of EZH2 inhibitor (GSK126) treatment, and the combination of both treatments in a DLBCL cell line, OCI-Ly1.

FIG. 11 shows the CTG signal relative to DMSO on Day 3 of JIB04 and Day 6 of EZH2 inhibitor (GSK126) treatment, and the combination of both treatments in a DLBCL cell line, OCI-Ly1.

FIG. 12 shows a proliferation assay following treatment with the indicated concentrations of JIB04 in a DLBCL cell line, OCI-Ly1.

FIG. 13 shows that JIB04 in the trans configuration (E isomer) is more is effective at reducing CTG signal relative to the cis configuration (Z isomer).

FIG. 14 shows the CTG signal relative to DMSO following JIB04 treatment, EED226 (an structural inhibitor of the PRC2 component, Novartis), and the combination of both treatments in a DLBCL cell line, OCI-Ly1. Viability is measured.

FIG. 15 shows a synergistic effect of a combined treatment of JIB04 and THZ-1 (a CDK7 inhibitor at a low dose, as compared to treatment of low dose JIB04 or THZ-1 alone).

FIG. 16 shows that JIB04 potency (as measured by assessing CTG signal relative to DMSO) is increased when combined with Ibrutinib, as compared to JIB04 treatment alone.

FIG. 17 is a line graph showing the dose response of JIB04 in various cancer cell types.

FIGS. 18A and 18B show KDM4 inhibition results in reduction in tumor burden within a short window of treatment. Pre-treatment (FIG. 18A) and 2 weeks post treatment (FIG. 18) with JIB04 at 30 mg/kg, 3 times weekly.

FIG. 19 shows a survival curve of mice at the indicated time post treatment with JIB04. Treated mice have increased survival as compared to vehicle treated mice.

FIG. 20 shows that KDM4 inhibition induces the downregulation of Ikaros family members and proximal components of BCR signaling pathway such as SYK. This demonstrates that targeting KDM4 can be a viable therapeutic option for cancers that show over-expression of Ikaros family members, such as IKZF1 and IKZF3.

FIG. 21A-21C show KDM4 inhibition induces the expression of extra-lineage transcription factors: including those that can inhibit Wnt-beta catenin signaling. FIG. 21A shows upregulation of transcription factors that contribute to inhibition of Wnt signaling (shown with square brackets and arrows). FIG. 21B is Venn diagram showing 51 genes show upregulation and gain activating epigenetic modifications. Analysis of these 51 genes by GSEA (FIG. 21C) shows that the top most upregulated GSEA category are the genes that get upregulated by inhibiting Wnt-beta catenin signaling. Other categories include regulation of cell death, Smad2-3 signaling pathway.

FIG. 22 is a line graph showing DLBCL cell lines are sensitive to Pyrvinium but not to Porcupine inhibitors.

FIG. 23 shows that that high expression of KDM4C in DLBCL patients correlated with poor prognosis.

### DETAILED DESCRIPTION

As described herein, the inventors have discovered inter alia that sensitivity of cancer cells to certain treatments (e.g., treatments that target KDM4/KDM5 family members) is dependent on whether the cancer cell contains one or more of the following:
(i) at least one mutation in an epigenetic modifier selected from the group consisting of EZH2, KMT2D, CREBPP, and EP300;
(ii) over-expression of at least one Ikaros family member;
(iii) over-expression of KDM4A and/or KDM4C; and/or
(iv) at least one mutation in canonical Wnt signaling.

Without wishing to be bound by a theory, a mutation in one of the above-noted epigenetic modifiers, i.e., EZH2, KMT2D, CREBPP, and/or EP300, can inhibit the pro-apoptotic mechanisms in the cancer cell. The inventors have discovered inter alia that targeting KDM4 and KDM5 family members can suppress this pro-apoptotic inhibition and this can sensitize cancer cells harboring these mutations to these treatments. Thus, targeting KDM4/KDM5 family members in cells harboring a mutation in at least one of epigenetic modifiers selected from EZH2, KMT2D, CREBPP, and/or EP300 gene promotes cancer cell apoptosis. Accordingly, provided herein are methods of treating cancer that relate to the mutations in these epigenetic modifiers.

Inventors have discovered inter alia that an exemplary compound targeting KDM4/KDM5, i.e., JIB-04, downregulates the level of key transcription factors that regulate cell fate. For example, the exemplary compound, JIB-04, downregulates levels of members of Ikaros family, such as IKZF1 and IKZF3. Thus targeting KDM4/KDM5 family members in cells having over-expression of an Ikaros family member, such as IKZF1 and/or IKZF3 can promote cancer cell apoptosis. Accordingly, provided herein are methods of treating cancer that relate to over-expression of an Ikaros family member, such as IKZF1 and/or IKZF3.

As the exemplary compound, JIB-04, targets KDM4A and KDM4C in cells which are sensitive to the compound. Thus, targeting KDM4/KDM5 family members in cells having over-expression of KDM4A and/or KDM4C can promote cancer cell apoptosis. Accordingly, provided herein are methods of treating cancer that relate to over-expression KDM4A and/or KDM4C.

Inventors have also discovered that the exemplary compound, JIB-04, inhibits canonical Wnt signaling. Thus, targeting KDM4/KDM5 family members in cells having a mutation in canonical Wnt-signaling can promote cancer cell apoptosis. Accordingly, provided herein are methods of treating cancer that relate to mutations in Wnt-signaling, such as activating Wnt-mutations.

Generally, the method comprises administering a compound targeting a KDM4/KDM5 family member to a subject in need of cancer treatment. As used herein, the term “targeting” with respect to targeting of a KDM4/KDM5 family member refers to modulating an activity of said family member. Further, “modulating” includes inhibiting or activating said family member. In some embodiments, the compound inhibits a KDM4/KDM5 family member.

Histone methylation is known to occur on the lysine residues of histones 3 and 4 (H3, H4), and the linker histone H1, isotype 4 (H1.4). On H3, four N-terminal lysine residues (K4, K9, K27, K36) and two structural residues (K56, K79) are able to be methylated [1, 7-10]. The linker histone H1.4, which is associated with intergenic regions of the genome, can also be methylated at lysine 26 (H1.4K26). At these histone lysine residues, methyltransferases and demethylases can, respectively, add or remove mono- (me1), di- (me2), or trimethyl (me3) marks, the degree of which alters chromatin compaction and gene expression. Methylation of H3K4, H3K36 and H3K79 is generally associated with gene activation, while methylation of H3K9, H3K27, H3K56, H4K20 and H1.4K26 is linked to transcriptional repression.

Demethylases are a class of enzymes that remove methyl (CH₃—) groups from nucleic acids, proteins (in particular histones), and other molecules. Demethylase enzymes are important in epigenetic modification mechanisms. Demethylase proteins can alter transcriptional regulation of the genome by controlling the methylation levels that occur on DNA and histones and, in turn, regulate the chromatin state at specific gene loci within organisms. Histone demethylase proteins have a variety of domains that serve different functions. These functions include binding to the histone (or sometimes the DNA on the nucleosome), recognizing the correct methylated amino acid substrate and catalyzing the reaction, and binding cofactors. There are several families of histone demethylases, which act on different substrates and play different roles in cellular function. For example, families of histone demethylases include KDM4 (e.g., KDM4A, KDM4B, KDM4C, and KDM4D) and KDM5 (e.g., KDM5A, KDM5B, KDM5C, KDM5D) families.

In some embodiments the various aspects disclosed herein, the compound targeting a KDM4/KDM5 family member is an inhibitor of a KDM4 family member. Members of the KDM family include includes KDM4A (SEQ ID NO: 1), KDM4B (SEQ ID NO: 2), KDM4C (SEQ ID NO: 3), and KDM4D (SEQ ID NO: 4). These are also referred to as JMDM3A/JMJD2A, JMDM3B/JMJD2B, JMDM3C/JMJD2C, and JMDM3DJMJD2D, respectively. These enzymes can act on di- and trimethylated H3K9, H3K36, H1K26 and catalyze the removal of methyl group(s) from histone lysine residues to epigenetically regulate chromatin structure and gene expression. KDM4 expression is tightly regulated to insure proper function in diverse biological processes, such as cellular differentiation. Mounting evidence has shown that disrupting KDM4 expression is implicated in the establishment and progression of multiple diseases including cancer. In particular, genomic regions encoding the KDM4A, B and C genes are often amplified, disrupting normal cellular proliferation. KDM4B and KDM4C have roles in tumorigenesis, and the role of KDM4D is unknown. The KDM4 family of proteins has been linked to malignant transformation. Specifically, KDM4C amplification has been documented in oesophageal squamous carcinomas, medulloblastomas and breast cancers; amplification of KDM4B has also been found in medulloblastomas. Other gene expression data has also suggested KDM4A, KDM4B, and KDM4C are over-expressed in prostate cancer.

As described herein, “KDM4A,” “Lysine-specific demethylase 4A,” or “JMJD2A” refers to a H3K9/36me3 lysine demethylase of the Jumonji domain 2 (JMJD2) family which converts specific trimethylated histone residues to the dimethylated form. KDM4A encodes a polypeptide having a JmjN domain, JmjC domain, two TUDOR domains, and two PHD-type zinc fingers.

KDM4A activity refers to the removal of a methyl from a trimethylated target histone to produce a dimethylated histone. Assays for measuring the activity of KDM4A are known in the art. Non-limiting examples of assays for KDM4A activity can include, MALDI-TOF spectrometry, and immunblotting or immunofluorescence microscopy with antibodies specific for tri and dimethylated histone targets, e.g. as described in Whetstine et al. Cell 2006 3:467-481; which is incorporated by reference herein in its entirety.

Specifically, the KDM4C gene encodes lysine-specific demethylase 4C. This gene is a member of the Jumonji domain 2 (JMJD2 or also known as JMDM3C) family and encodes a protein with one JmjC domain, one JmjN domain, two PHD-type zinc fingers, and two Tudor domains. This nuclear protein functions as a trimethylation-specific demethylase, converting specific trimethylated histone residues to the dimethylated form. Chromosomal aberrations and increased transcriptional expression of this gene are associated with esophageal squamous cell carcinoma.

The compound targeting a KDM4/KDM5 family member can inhibit one of the KDM4 activities described above. For example, the compound can inhibit KDM4A, KDM4B, KDM4C or KDM4D. In some embodiments, the compound is a KDM4A and/or KDM4C inhibitor.

Non-limiting inhibitors of KDM4 family members include, but are not limited to, hydroxyquinoline (8HQ), prolyl hydroxylase domain 2 (PHD2), ML324, 3-((furan-2-ylmethyl)amino)pyridine-4-carboxylic acid, 3-(((3-methylthiophen-2-yl)methyl)amino)pyridine-4-carboxylic acid, Suv39H1, HP1, succinate and miRNA, such as miR23a, miR23b, miR200a, miR200b, miR200c, and miR137a or variants thereof. In some embodiments, the inhibitor of KDM4 family is the compound JIB04 or derivatives thereof. JIB04 is a pan-JumonjiC demethylase inhibitor, i.e. it inhibits two or more JmjC enzymes. The inventors have now discovered that JIB04 can also inhibit KDM4C. Structure of JIB04 is shown below.

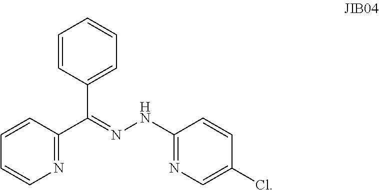 [SMILES: Clc1ccc(N/N=C(\c2ccccc2)c2ccccn2)nc1]

Members of the KDM5 family include KDM5A (SEQ ID NO: 5), KDM5B (SEQ ID NO: 6), KDM5C (SEQ 1D NO: 7), and KDM5D (SEQ ID NO: 8). These are also referred to as JAR1D1A/RBP2, JARID1B/PLU-1, JARID1C/SMCX, and JARID1DiSMCY, respectively. These enzymes remove tri- and di-methylations of lysine 4 of histone H3-modifications that occur at the start site of transcription in actively transcribed genes. KDM5 protein family appear to play key developmental functions. The deletion of the JmjC domain of retinoblastoma binding protein related 2 (RBR-2) in C. elegans express defects in vulva formation. Mutations to the JmjC domain in Drosophila causes either lethal effects on larval or many developmental defects in those that survive. KDM5A in cell culture systems have also shown links to regulation of differentiation, mitochondrial function, cell cycle progression. KDM5B and KDM5C have also shown to interaction with PcG proteins, which are involved in transcriptional repression. KDM5C mutations (found on the X-chromosome) have also been found in patients with X-linked mental retardation. Depletion of KDM5C homologs in D. rerio have shown brain-patterning defects and neuronal cell death. KDM5 family members of histone demethylases have a prevalent role in human cancer. In particular, KDM5A (JARID1A/RBP2) and KDM5B (JARID1B/PLU1) contribute to cancer cell proliferation, reduce the expression of tumor suppressor genes, promote the development of drug tolerance and maintain tumor-initiating cells.

In some embodiments the various aspects disclosed herein, the compound targeting the KDM4/KDM5 family member is an inhibitor of a KDM5A or KDM5B. Non-limiting inhibitors of KDM5 family members include e.g., CPI-455 and KDM5-C70.

In some embodiment, the compound targeting KDM4/KDM5 family member inhibits KDM5A and/or KDM5B.

Compounds targeting a KDM4/KDM5 family member can also include nucleic acids. For example, nucleic acids that can bind with and reduce or inhibit expression of a nucleic acid encoding the family member. Without wishing to be bound by a theory, reduction or inhibition of the expression can inhibit activity of the family member. Exemplary nucleic acid for reducing or inhibiting expression of a KDM4/KDM5 family member include, but are not limited to, small interfering RNAs (siRNAs), short hairpin RNAs (shRNAs), antisense oligonucleotides, triplex forming oligonucleotide, and ribozymes.

The term “siRNA” refers to any non-endogenous and synthetic RNA duplex designed to specifically target a particular mRNA for degradation. Accordingly, “siRNA” refers to an RNA capable of down-regulating its target expression level via activation of the DICER complex. The term “mRNA” refers to a nucleic acid transcribed from a gene from which a polypeptide is translated, and can include non-translated regions such as a 5′UTR and/or a 3′UTR. An siRNA can include a 21 base-pair nucleotide sequence that is completely complementary to any sequence of an mRNA molecule, including translated regions, the 5′UTR, the 3′UTR, and sequences that include both a translated region and a portion of either 5′UTR or 3′UTR. In some embodiments, the siRNA comprises a sequence complimentary to at least a part of a sequence selected from the group consisting of SEQ ID NOs: 1-6 and 8. In some further embodiments, the siRNA comprises a sequence complimentary to at least a part of a sequence selected from the group consisting of SEQ ID NOs: 1 and 2.

The term “shRNA” refers to any non-endogenous artificial RNA molecule with a tight hairpin turn that can be used to silence target gene expression via RNA interference. Accordingly, “shRNA” refers to an RNA capable of down-regulating its target expression level via activation of Drosha. An shRNA can include a nucleotide sequence that is completely complementary to any sequence of an mRNA molecule, including translated regions, the 5′UTR, the 3′UTR, and sequences that include both a translated region and a portion of either 5′UTR or 3′UTR. In some embodiments, the shRNA comprises a sequence complimentary to at least a part of a sequence selected from the group consisting of SEQ ID NOs: 1-6 and 8. In some further embodiments, the shRNA comprises a sequence complimentary to at least a part of a sequence selected from the group consisting of SEQ ID NOs: 1 and 2.

The term “oligonucleotide” as used herein refers to an oligomer or polymer of ribonucleic acid (RNA) or deoxyribonucleic acid (DNA) or mimetics thereof, as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleomonomers covalently coupled to each other by linkages (e.g., phosphodiesters) or substitute linkages. The term “antisense oligonucleotides” refers a 15-20 base-pair polymer comprising chemically-modified deoxynucleotides. Its sequence in antisense (3′-5′) such that it is complementary to its target mRNA. Accordingly, “antisense oligonucleotides” refers to a polymer that, upon mRNA binding prevents synthesis of the target and promotes degradation of the target. In some embodiments, the antisense oligonucleotide comprises a sequence complimentary to at least a part of a sequence selected from the group consisting of SEQ ID NOs: 1-6 and 8. In some further embodiments, the antisense oligonucleotide comprises a sequence complimentary to at least a part of a sequence selected from the group consisting of SEQ ID NOs: 1 and 2.

In some embodiments of the various aspects disclosed herein, means for targeting the KDM4/KDM5 family member can be a CRISPR/Cas system

Cyclin dependent kinases (CDK) are a family of protein kinases involved in regulating the cell cycle. Aberrant expression of CDKs has been linked to numerous cancers, and agents that target CDK activity have been an attractive target for development of anti-tumor therapies. Data presented herein show a synergistic effect between JIB04 and THZ-1, a CDK7 inhibitor, when administered in combination, as compared to JIB04 or THZ-1 administration alone. Specifically, co-administration of 10 nM JIB04 and 100 nm THZ-1 was effective at inducing cell death, whereas administration of 10 nM JIB04 alone was not.

Accordingly, in some embodiments of the various aspects disclosed herein, the method further comprises administering a cyclin-dependent kinase inhibitor, for example, a CDK7 inhibitor. Without limitations, the CDK inhibitor can be administered to the subject prior to, simultaneously with or after administering the compound targeting the KDM4/KDM5 family member. When the CDK inhibitor is administered simultaneously with the compound targeting the KDM4/KDM5, the CDK inhibitor (e.g., a CDK7 inhibitor, such as THZ-1) and the compound targeting the KDM4/KDM5 family member can be formulated as a single composition.

In some embodiments of the various aspects disclosed herein, a CDK inhibitor and a compound targeting the KDM4/KDM5 family member are co-administered to the subject, where at least one of the CDK inhibitor and the compound targeting the KDM4/KDM5 family member is administered in an amount that is not effective to treat cancer when administered alone. For example, the CDK inhibitor and the compound targeting the KDM4/KDM5 family member are co-administered to the subject, where the CDK inhibitor is administered in an amount that is not effective to treat cancer when the CDK inhibitor is administered alone. In another non-limiting example, the CDK inhibitor and the compound targeting the KDM4/KDM5 family member are co-administered to the subject, where the compound targeting the KDM4/KDM5 family member is administered in an amount that is not effective to treat cancer when the compound is administered alone.

In still another non-limiting example, the CDK inhibitor and the compound targeting the KDM4/KDM5 family member are co-administered to the subject, where the CDK inhibitor and the compound targeting the KDM4/KDM5 family member are administered in an amount that is not effective to treat cancer when the CDK inhibitor and the compound targeting the KDM4/KDM5 family member are administered alone.

In some embodiments of the various aspects disclosed herein, the CDK inhibitor is a CDK7 inhibitor. CDK7 is a member of the cyclin-dependent kinase family that functions to regulate the cell cycle, e.g., to regulate the G1 phase of the cell cycle. CDK7 forms a trimeric complex with cyclin H and MAT1 to function as a Cdk-activating kinase (CAK). The activity of CDKs is regulated by multiple mechanisms such as positive and negative phosphorylation, binding of regulatory proteins like cyclins and CDK inhibitors. Exemplary CDK7 inhibitors include, but are not limited to, BS-181 HCl, PHA-793887, SNS-032, Milciclib, Flavopiridol (Alvocidib), Flavopiridol (Alvocidib) HCl, AT7519, P276-00, and THZ-1.

Recently, selective CDK7 inhibitors have been identified, which target only CDK7 or target CDK7 with a higher inhibitory activity than any other CDK. Thus, in some embodiments, the CDK inhibitor is a selective CDK7 inhibitor selected from those disclosed in WO2015/058163, WO 2015/154022, WO 2015/154038, WO 2015/154039, WO 2015/058140 and WO 2014/063068, contents of all which are incorporated herein by reference in their entirety.

In some embodiments of the various aspect disclosed herein, the CDK7 inhibitor is THZ-1. For example, the compound targeting a KDM4/KDM5 family member is JIB04, and the CDK7 inhibitor is THZ-1.

In some embodiments of the various aspects disclosed herein, the compound targeting a KDM4/KDM5 family member is JIB-04 and CDK7 inhibitor is THZ-1, where at least one of JIB-04 and THZ-1 is administered in an amount that is not effective to treat cancer when the JIB-04 or THZ-1 is administered alone. For example, the compound targeting a KDM4/KDM5 family member is JIB-04 and CDK7 inhibitor is THZ-1, where JIB-04 is administered in an amount that is not effective to treat cancer when the JIB-04 is administered alone. In another example, the compound targeting a KDM4/KDM5 family member is JIB-04 and CDK7 inhibitor is THZ-1, where THZ-1 is administered in an amount that is not effective to treat cancer when THZ-1 is administered alone. In still another example, the compound targeting a KDM4/KDM5 family member is JIB-04 and CDK7 inhibitor is THZ-1, where both of JIB-04 and THZ-1 are administered in an amount that is not effective to treat cancer when the JIB-04 or THZ-1 are administered alone.

B-cell receptor (BCR) is important for normal B-cell development and is associated in the development of the most common B-cell malignancies. BCR serves as an antigen receptor and regulates multiple cellular processes, including proliferation, differentiation, apoptosis and cell migration. The BCR consists of a transmembrane immunoglobin (Ig), receptor associated with the Ig-alpha (CD79a) and Ig-beta (CD79b) heterodimers. Once the antigen binds to the receptor, the tyrosine kinases LYN and SYK initiate a signaling cascade that involves downstream kinases, adapter molecules and generation of second messengers. BCR signaling is critical in both GCB and ABC type DLBCL, and inventors have discovered that both of those lines are sensitive to JIB04. In addition, presented herein are data showing that the potency of JIB04 (e.g., JIB04's capacity to induce cell death in a target cell, e.g., a cancer cell) is significantly increased when administered in combination with an inhibitor of Bruton's Tyrosine Kinase (BTK), as compared to JIB04 administration alone. Bruton's Tyrosine Kinase is one of the signaling molecules that is essential in the BCR pathway.

Accordingly, in some embodiments of the various aspects disclosed herein, the method further comprises administering an inhibitor of BCR signaling, for example an inhibitor of BTK, to the subject. Without limitations, the inhibitor of BCR signaling can be administered to the subject prior to, simultaneously with or after administering the compound targeting the KDM4/KDM5 family member. When the inhibitor of BCR signaling is administered simultaneously with the compound targeting the KDM4/KDM5, the inhibitor of BCR signaling (e.g., an inhibitor of BTK, such as Ibrutimb) and the compound targeting the KDM4/KDM5 family member can be formulated as a single composition.

In some embodiments of the various aspects disclosed herein, BCR signaling inhibitor and a compound targeting the KDM4/KDM5 family member are co-administered to the subject, where at least one of the BCR signaling inhibitor and the compound targeting the KDM4/KDM5 family member is administered in an amount that is not effective to treat cancer when administered alone. For example, the BCR signaling inhibitor and the compound targeting the KDM4/KDM5 family member are co-administered to the subject, where the BCR signaling inhibitor is administered in an amount that is not effective to treat cancer when the BCR signaling inhibitor is administered alone. In another non-limiting example, the BCR signaling inhibitor and the compound targeting the KDM4/KDM5 family member are co-administered to the subject, where the compound targeting the KDM4/KDM5 family member is administered in an amount that is not effective to treat cancer when the compound is administered alone.

In still another non-limiting example, the BCR signaling inhibitor and the compound targeting the KDM4/KDM5 family member are co-administered to the subject, where the BCR signaling inhibitor and the compound targeting the KDM4/KDM5 family member are administered in an amount that is not effective to treat cancer when the BCR signaling inhibitor and the compound targeting the KDM4/KDM5 family member are administered alone.

BTK plays a critical role in B-cell activation and mast cell activation via, e.g., the high affinity IgE receptor. BTK contains a PH domain that binds phosphatidylinositol (3,4,5)-trisphosphate (PIP3). PIP3 binding induces BTK to phosphorylate phospholipase C, which in turn hydrolyzes PIP2, a phosphatidylinositol, into two second messengers, inositol triphosphate (IP3) and diacylglycerol (DAG), which then modulates the activity of downstream proteins during B-cell signaling. Accordingly, in some embodiments of the various aspects disclosed herein, the inhibitor of BCR signaling is an inhibitor of Bruton's Tyrosine Kinase, i.e., a BTK inhibitor. Exemplary BTK inhibitors include, but are not limited to, Spebrutinib (CC-292, AVL-292), CNX-774, ONO-4059 (GS-4059) HCl, and Ibrutinib. In some embodiments, the BTK inhibitor is Ibrutinib. For example, the compound targeting a KDM4/KDM5 family member is JIB04, and the BTK inhibitor is Ibrutinib.

In some embodiments of the various aspects disclosed herein, the compound targeting a KDM4/KDM5 family member is JIB-04 and the BTK inhibitor is Ibrutinib, where at least one of JIB-04 and Ibrutinib is administered in an amount that is not effective to treat cancer when the JIB-04 or Ibrutinib is administered alone. For example, the compound targeting a KDM4/KDM5 family member is JIB-04 and the BTK inhibitor is Ibrutinib, where JIB-04 is administered in an amount that is not effective to treat cancer when the JIB-04 is administered alone. In another example, the compound targeting a KDM4/KDM5 family member is JIB-04 and the BTK inhibitor is Ibrutinib, where Ibrutinib is administered in an amount that is not effective to treat cancer when Ibrutinib is administered alone. In still another example, the compound targeting a KDM4/KDM5 family member is JIB-04 and the BTK inhibitor is Ibrutinib, where both of JIB-04 and Ibrutinib are administered in an amount that is not effective to treat cancer when the JIB-04 or Ibrutinib are administered alone.

Pyrvinium, an FDA-approved anti helminth, can delay or inhibit tumor cell proliferation in cancer models including colon, breast, lung and prostate cancer, and some hematological malignancies. Pyrvinium has also been found to inhibit canonical Wnt-signaling. In some embodiments of the various aspects disclosed herein, Pyrvinium and a compound targeting the KDM4/KDM5 family member are co-administered to the subject, where at least one of Pyrvinium and the compound targeting the KDM4/KDM5 family member is administered in an amount that is not effective to treat cancer when administered alone. For example, Pyrvinium and the compound targeting the KDM4/KDM5 family member are co-administered to the subject, where Pyrvinium is administered in an amount that is not effective to treat cancer when Pyrvinium is administered alone. In another non-limiting example, Pyrvinium and the compound targeting the KDM4/KDM5 family member are co-administered to the subject, where the compound targeting the KDM4/KDM5 family member is administered in an amount that is not effective to treat cancer when the compound is administered alone.

In some embodiments of the various aspects disclosed herein, the compound targeting a KDM4/KDM5 family member is JIB-04 and where at least one of JIB-04 and Pyrvinium is administered in an amount that is not effective to treat cancer when the JIB-04 or Pyrvinium is administered alone. For example, JIB-04 and Pyrvinium are co-administered to the subject, where JIB-04 is administered in an amount that is not effective to treat cancer when the JIB-04 is administered alone. In another example, JIB-04 and Pyrvinium are co-administered to the subject, where Pyrvinium is administered in an amount that is not effective to treat cancer when Pyrvinium is administered alone. In still another example, JIB-04 and Pyrvinium are co-administered to the subject, where both of JIB-04 and Pyrivinium are administered in an amount that is not effective to treat cancer when the JIB-04 or Pyrivinium are administered alone.

As discussed herein, inventors have discovered inter alia that sensitivity of cancer cells to treatments that target KDM4/KDM5 family members can be enhanced when the cells have a mutation in at least one of the epigenetic modifiers selected from the group consisting of EZH2, KMT2D, CREBPP, and EP300. Enhancer of zeste homolog 2 (EZH2) is a histone-lysine N-methyltransferase enzyme (EC 2.1.1.43) encoded by EZH2 gene, that participates in DNA methylation and, ultimately, transcriptional repression. EZH2 catalyzes the addition of methyl groups to histone H3 at lysine 27, by using the cofactor S-adenosyl-L-methionine. Methylation activity of EZH2 facilitates heterochromatin formation thereby silences gene function. Remodeling of chromosomal heterochromatin by EZH2 is also required during cell mitosis. EZH2 is the functional enzymatic component of the Polycomb Repressive Complex 2 (PRC2), which is responsible for healthy embryonic development through the epigenetic maintenance of genes responsible for regulating development and differentiation. EZH2 is responsible for the methylation activity of PRC2, and the complex also contains proteins required for optimal function (EED, SUZ12, JARID2, AEBP2, RbAp46/48, and PCL). Mutation or over-expression of EZH2 has been linked to many forms of cancer. EZH2 inhibits genes responsible for suppressing tumor development, and blocking EZH2 activity may slow tumor growth. EZH2 has been targeted for inhibition because it is upregulated in multiple cancers including, but not limited to, breast, prostate, melanoma, and bladder cancer. Mutations in the EZH2 gene are also associated with Weaver syndrome, a rare congenital disorder, and EZH2 is involved in causing neurodegenerative symptoms in the nervous system disorder, ataxia telangiectasia.

Histone-lysine N-methyltransferase 2D (KMT2D), also known as MLL4 and sometimes MLL2 in humans and Mll4 in mice, is a major mammalian histone H3 lysine 4 (H3K4) mono-methyltransferase. It is part of a family of six Set1-like H3K4 methyltransferases that also contains KMT2A (or MLL1), KMT2B (or MLL2), KMT2C (or MLL3), KMT2F (or SET1A), and KMT2G (or SET1B). KMT2D is a large protein over 5,500 amino acids in size and is widely expressed in adult tissues. The protein co-localizes with lineage determining transcription factors on transcriptional enhancers and is essential for cell differentiation and embryonic development. It also plays critical roles in regulating cell fate transition, metabolism, and tumor suppression. Mutations in KMT2D have been associated with Kabuki Syndrome, congenital heart disease, and various forms of cancer.

CREB-binding protein (CREBBP) is a protein encoded by the CREBBP gene that carries out its function by activating transcription, where interaction with transcription factors is managed by one or more CREB domains: the nuclear receptor interaction domain (RID), the CREB and MYB interaction domain (KIX), the cysteine/histidine regions (TAZ1/CH1 and TAZ2/CH3) and the interferon response binding domain (IBiD). CREBBP has intrinsic histone acetyltransferase activity and also acts as a scaffold to stabilize additional protein interactions with the transcription complex. CREBBP acetylates both histone and non-histone proteins. This protein shares regions of very high-sequence similarity with protein EP300 in its bromodomain, cysteine-histidine-rich regions, and histone acetyltransferase domain. The CREB protein domains, KIX, TAZ1 and TAZ2, each bind tightly to a sequence spanning both transactivation domains 9aaTADs of transcription factor p53. This gene is ubiquitously expressed and is involved in the transcriptional coactivation of many different transcription factors. It plays critical roles in embryonic development, growth control, and homeostasis by coupling chromatin remodeling to transcription factor recognition.

Histone acetyltransferase p300 (EP300) also known as p300 HAT or E1A-associated protein p300′ (where E1A=adenovirus early region 1A) is an enzyme that, in humans, is encoded by the EP300 gene. It functions as histone acetyltransferase that regulates transcription of genes via chromatin remodeling. This enzyme plays an essential role in regulating cell growth and division, prompting cells to mature and assume specialized functions (differentiate), and preventing the growth of cancerous tumors. The EP300 protein appears to be critical for normal development before and after birth.

In some embodiments of the various aspects disclosed herein, a mutation is present in at least one of the epigenetic modifiers selected from the group consisting of EZH2, KMT2D, CREBPP, and EP300. Non-limiting examples of mutations include missense mutation, nonsense mutation, insertion mutation, deletion mutation, duplication mutation, frameshift mutation, point mutation, amorphic mutation, antimorphic mutation, hypermorphic mutation, gain of function mutation, loss of function mutation, hypomorphic mutation, neomorphic mutation, or null mutation. Non-limiting causes of mutations can be spontaneous mutations (molecular decay), mutations due to error-prone replication bypass of naturally occurring DNA damage (also called error-prone translesion synthesis), errors introduced during DNA repair, or induced mutations caused by mutagens.

Methods, reagents and systems for determining presence of mutations are well known For example, the presence of the mutation can be determined using an assay selected from the group consisting of hybridization, sequencing, high-throughput sequencing, PCR, qPCR, exome capture, FISH, RFLP, and immunochemical detection methods.

As described herein, one or more of the epigenetic modifiers selected from the group consisting of EZH2, KTM2D, CREBBP, and EP300 can comprise a mutation. When two or more of these epigenetic modifiers comprise a mutation, all the epigenetic modifiers can comprise the same type of mutation, different types of mutations or some combination of same or different mutations. In other words, when two or more of these epigenetic modifiers comprise a mutation, each mutation can be selected independently from the group consisting of missense mutation, nonsense mutation, insertion mutation, deletion mutation, duplication mutation, frameshift mutation, point mutation, amorphic mutation, antimorphic mutation, hypermorphic mutation, gain of function mutation, loss of function mutation hypomorphic mutation, neomorphic mutation, and null mutation.

Without limitation, a subject can have one mutation in any epigenetic modifier selected from the group consisting of EZH2, KTM2D, CREBBP, and EP300. For example, the subject can have a point mutation in EZH2, KTM2D, CREBBP or EP300. In another example, the subject can have a mutation in any two epigenetic modifiers selected from the group consisting of EZH2, KTM2D, CREBBP, and EP300. For example, a point mutation in CREBBP and a missense mutation in EP300). In yet another example, the subject can have a mutation in any three epigenetic modifiers selected from the group consisting of EZH2, KTM2D, CREBBP, and EP300. For example, a point mutation in CREBBP, a missense mutation in EP300, and a frameshift mutation in EZH2. In still another example, the subject can have a mutation in all four of the epigenetic modifiers. For example, a point mutation in CREBBP, a missense mutation in EP300, a frameshift mutation in EZH2, and a deletion mutation in KTM2D.

Further, the mutation can be an activating mutation, e.g., a gain of function mutation, or the mutation can be a deactivating mutation, e.g., a loss of function mutation. For example, the mutation in KMT2D, EP300, and/or CREBPP can be a loss of function mutation. A mutation in EZH2 can be a gain of function mutation.

The Wnt family of glycoproteins control a variety of developmental processes including cell fate specification, proliferation, polarity and migration. Consequently, the Wnt pathway is instrumental in ensuring proper tissue development in embryos and tissue maintenance in adults. There are at least three signaling pathways involved in the Wnt signal transduction process. The canonical (or B-catenin dependent) Wnt pathway was discovered first and has been studied most. In the absence of a Wnt signal, the transcriptional activator B-catenin is a phosphorylated intracellular multi-protein complex which is subsequently degraded. Within this complex the AXIN and adenomatous polyposis coli (APC) proteins form a scaffold that facilitates B-catenin phosphorylation by casein-kinasela (CK1a) and glycogen synthase kinase 33 (GSK-3B). Phosphorylated B-catenin is subsequently ubiquitinylated, resulting in its degradation in the proteasome. When Wnt signaling is inactive and therefore levels of free B-catenin are low, DNA-binding T-cell factor/lymphoid enhancer factor (TCF/LEF) proteins interact with transcriptional repressors to block Wnt target gene expression in the nucleus. Binding of Wnt molecules to FZD-LRP receptor complexes at the membrane leads to a cascade of events that lead to the inactivation of the 13-catenin destruction complex. This allows B-catenin to accumulate and enter the nucleus where it interacts with members of the Tcf/Lef family and converts the Tcf proteins into potent transcriptional activators by recruiting co-activator proteins ensuring efficient activation of Wnt target genes.

Blocking canonical Wnt activity in colorectal and other Wnt deregulated cancers has been shown to cause cell cycle arrest in G1 and this is a crucial step in inhibiting tumor cell growth (van de Wetering et al., Cell 111: 241-250, 2002; and Sukhdeo et al., Proc. Natl. Acad. Sci. USA 104: 7516-7521, 2007). In recent years, several classes of small-molecules have been shown to act as Wnt inhibitors. These drugs exert their inhibitory effects at various levels of the Wnt signaling pathway. Small molecules, interfering with nuclear TCF/I3-catenin binding and with the cyclic AMP response element-binding protein (CBP), have been identified and described (Emami et al., Proc. Natl. Acad. Sci. USA 101: 12682-12687, 2004; and Lepourcelet M et al., Cancer Cell 5: 91-102, 2004). Topo Ha and PARP-1 (Shitashige et al., Cancer Sci. 99: 631-637, 2008) or TBP, BRG1, BCL9, pygopus and Hyrax (Barker et al. supra) have been proposed to be potential targets for inhibiting canonical Wnt signaling. Recently, two groups of chemical substances (IWR-1 and XAV939) have been identified which stabilize the destruction complex (Chen et al., Nat. Chem. Biol. 5: 100-107, 2009; and Huang et al., Nature: 461: 614-620, 2009). By blocking the PARP domain of Tankyrase, XAV939 and IWR-1 are thought to alter the PARsylation and ubiquitination of AXIN2 that results in its increased stability and in inhibition of canonical Wnt signaling. Since elevated levels of 13-catenin in the nucleus are a common feature of abnormal canonical Wnt signaling, down-regulation of canonical Wnt activity by reducing the presence of 13-catenin represents a potential therapeutic strategy.

Canonical Wnt signaling is over-activated in a variety of tumors where it plays a central role in cell growth and tumor progression (Barker et al., Nat. Rev. Drug. Discov. 5: 997-1014, 2006; Grigoryan et al., Genes Dev. 22: 2308-2341, 2008; and Shitashige et al., Cancer Sci. 99: 631637, 2008). About 90% of sporadic colon cancers show aberrant Wnt signaling (Liu et al., Nat. Genet. 26: 146-147, 2000; and Morin et al., Science 275: 1787-1790, 1997), while all pancreatic adenocarcinomas exhibit alterations in Wnt/Notch signaling (Jones et al., Science 321: 18011806, 2008).

Wnt activating mutations are present in a variety of cancers including gastric cancer, hepatocellular carcinoma, Wilms tumor of the kidney, medulloblastoma, melanoma, non-small cell lung cancer, ovarian endometriod cancer, anaplastic thyroid cancer, pancreas adenocarcinoma, and prostate cancer. Mutations in the adenomatous polyposis coli gene (APC), 13-catenin, or Axin genes lead to accumulation of nuclear 13-catenin and such mutations are frequently associated with colon cancer (Morin et al. supra). Furthermore, alterations in extracellular proteins which silence Wnt signaling including secreted frizzled related proteins (SFRPs) (Suzuki et al., Nat. Genet 36: 417-422, 2004), Dickkopf (Dkk) (Aguilera et al., Oncogene 25: 4116-4121, 2006) and members of the Wnt inhibitor factor (WIF) family (Mazieres et al., Cancer Res. 64: 4717-4720, 2004) can also lead to abnormal pathway activity (Polakis, Curr. Opin. Genet. Dev. 17: 45-51, 2007). Accordingly, in some embodiments of the various aspects disclosed herein, a mutation in canonical Wnt-signaling can be a Wnt activating mutation, also referred to as an activating Wnt-mutation herein.

As discussed herein, inventors have discovered that sensitivity of cancer cells to treatments that target KDM4/KDM5 family members can be enhanced when the cell comprises at least one mutation in the canonical Wnt-signaling pathway. Accordingly, in some embodiments of the various aspects disclosed herein, the cancer to be treated is a Wnt-dependent cancer. For example, the subject to be treated has at least one mutation in canonical Wnt signaling. As used herein, “Wnt-dependent” refers to a cancer that is driven or caused by increased canonical Wnt activity. “Wnt-dependent” cancers can contain, e.g., mutations within the genes or gene products that make up the Wnt signaling pathway (e.g., Axin, β-Catenin, APC, or LRP-5) that results in its abnormal activation. One skilled in the art will be able to determine if a cancer is Wnt-dependent via, e.g., assays that measure activation of canonical Wnt target genes, e.g., TCG/LEF. Exemplary Wnt-dependent cancers include, but are not limited to, hepatocellular cancer, medulloblastoma, colorectal cancer, gastric cancer, lymphoma, leukemia, breast cancer, parathyroid cancer, and Wilm's tumor. Inactivation of canonical Wnt signaling can be indirect. For example, administration of a compound that targets KDM4-KDM5 family members can activate non-canonical Wnt signaling, which then acts to suppress canonical Wnt signaling. Additionally, data presented herein show that an exemplary compound targeting a KDM4/KDM5 family member, e.g., JIB-04, shows that the compound likely up regulates non-canonical Wnt pathway to inhibit canonical Wnt/beta-catenin pathway. It is noted that colon cancer cell lines HCT 116 and Colo205, both have mutations in canonical Wnt signaling. HCT116 also has mutations in EP300 and KMT2D, while Colo205 does not.

In some embodiments, the method for treating cancer comprises administering a therapeutically effective amount of JIB04 to a subject in need thereof, wherein the subject has at least one mutation in canonical Wnt signaling. In some further embodiments, the cancer is selected form the group consisting of DLBCL and colon cancer.

As also discussed herein, inventors have discovered inter alia that sensitivity of cancer cells to treatments that target KDM4/KDM5 family members can be enhanced when at least one Ikaros transcription factor family member is over-expressed, e.g., protein, gene-expression or activity, in the cells. Ikaros family members all belong to the zinc-finger DNA-binding proteins associated with chromatin remodeling. IKZF1 (IKAROS zinc finger 1; also called Ikaros) functions as a regulator of lymphocyte differentiation. Several alternatively spliced transcript variants encoding different isoforms have been described for this gene. All isoforms share a common C-terminal domain, which contains two zinc finger motifs that are required for hetero- or homo-dimerization and for interactions with other proteins. The isoforms, however, differ in the number of N-terminal zinc finger motifs that bind DNA and contain the nuclear localization signal, resulting in members with and without DNA-binding properties. Only few isoforms contain the requisite three or more N-terminal zinc motifs that confer high affinity binding to a specific core DNA sequence element in the promoters of target genes. The non-DNA-binding isoforms are largely found in the cytoplasm, and thought to function as dominant negative factors Overexpression of some dominant-negative isoforms have been associated with B-cell malignancies, such as acute lymphoblastic leukemia (ALL). IKZF2 (IKAROS zinc finger 2; also called Helios) forms homo- or hetero-dimers with other Ikaros family members, and is thought to function predominantly in early hematopoietic development. IKZF3 (IKAROS zinc finger 3; also called Aiolos) is a transcription factor that is important in the regulation of B lymphocyte proliferation and differentiation Both Ikaros and Aiolos can participate in chromatin remodeling Regulation of gene expression in B lymphocytes by Aiolos is complex as it appears to require the sequential formation of Ikaros homodimers, Ikaros/Aiolos heterodimers, and Aiolos homodimers. IKZF4 (IKAROS zinc finger 4; also called Eos) is expressed in lymphocytes and are implicated in the control of lymphoid development.

In some embodiments of the various aspects disclosed herein, at least one member of the Ikaros family is over-expressed in the subject. Without limitations, said over-expression can be an increased amount of the Ikaros family member (e.g., increased protein level) or increased amount of a nucleic acid encoding the Ikaros family member (e.g., DNA or mRNA). In some embodiments, the over-expression can be an increased activity level. For example, the Ikaros family member comprises a mutation that confers gain of function activity to the family member. In some embodiments of the various aspect disclose herein, the Ikaros family member is IKZF1 and/or IKZF3.

Exemplary methods for determining the over-expression of Ikaros family members include, but are not limited to, qPCR-based diagnostic tests to assay RNA (e.g., mRNA) encoding Ikaros family member, immunohistochemical assay (such as using antibodies against Ikaros family member) to determine protein levels, FISH-based assays to assay DNA amplification of Ikaros family members, and sequencing to assay for mutations that can confer gain of function activity.

In some embodiments, the method for treating cancer comprises administering a therapeutically effective amount of JIB04 to a subject in need thereof, wherein at least one member of Ikaros family is overexpressed in the subject. In some further embodiments, the cancer is selected form the group consisting of DLBCL and colon cancer.

As discussed herein, inventors have also discovered that sensitivity of cancer cells to treatments that target KDM4/KDM5 family members can be enhanced when at least one of KDM4A and KDM4C is over-expressed, e.g., protein, gene-expression or activity, in the cells. Without limitations, said over-expression can be an increased amount of KDM4A and/or KDM4C, or increased amount of a nucleic acid encoding KDM4A and/or KDM4C (e.g., DNA or mRNA). In some embodiments, the over-expression can be an increased activity level. For example, KDM4A and/or KDM4C comprises a mutation that confers gain of function activity, e.g., enhanced demethylation activity. Exemplary methods for determining the over-expression of KDM4A and KDM4C include, but are not limited to, qPCR-based diagnostic tests to assay RNA (e.g., mRNA) encoding KDM4A and/or KDM4C, immunohistochemical assay (such as using antibodies against KDM4A and/or KDM4C) to determine protein levels, FISH-based assays to assay DNA amplification of KDM4A and/or KDM4C, and sequencing to assay for mutations that can confer gain of function activity KDM4A and/or KDM4C.

In some embodiments, the method for treating cancer comprises administering a therapeutically effective amount of JIB04 to a subject in need thereof, wherein KDM4A and/KDM4C is overexpressed in the subject. In some further embodiments, the cancer is selected form the group consisting of DLBCL and colon cancer.

In some embodiments of the various aspects disclosed herein, the method can further comprise administering an additional anti-cancer therapy to the subject. For example, administering a standard of care chemotherapeutic to the subject. Non-limiting examples of a standard of care chemotherapeutics or other anti-cancer therapy can include radiation therapy, surgery, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above. Additional anti-cancer treatment can further include the use of radiation or radiation therapy. Further, the additional anti-cancer treatment can also include the use of surgical treatments.

In some embodiments of the various aspects disclosed herein, the treatment is administered to a subject currently receiving standard of care chemotherapeutics or other alternative anti-cancer treatments. Generally, cancer treatment may involve one or more of the treatment options, but not limited to surgery, radiation, chemotherapy, immunotherapy, targeted therapy and hormonal therapy. The single agent therapy or current combination therapies for the treatment of cancer cause side effects such as nausea, rashes, swelling, flu-like symptoms, fatigue, digestive tract problems, allergic reactions and immunosuppression. In some embodiments, the invention described herein provides a more effective treatment of cancer by administering one or more inhibitors of KDM4 or KDM5 family in combination with other cancer treatments. In some embodiments, the combination therapy induces additive or synergistic therapeutic effect. In some embodiments, the method described herein can reduce or prevent one or more adverse effects or toxicities associated with the administration of a chemotherapeutic agent or radiation therapy. In some embodiments, the method described herein can increase the anti-tumor activity of a chemotherapeutic agent or radiation therapy or increase the selective cytotoxicity of a chemotherapeutic agent.

The phrase “combination therapy” as described herein means administration of one or more inhibitors of KDM4 or KDM5 family and a therapeutic agent as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period. The time period may be in minutes, hours, days or weeks depending upon the combination selected.

Combination therapy includes administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be done, for example, by administering to the subject a single pill having a fixed ratio of each therapeutic agent or in multiple, single pills for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered may or may not be important.

Combination therapy also can mean the administration of one or more inhibitors of KDM4 or KDM5 family in further combination with other compounds and non-drug therapies, such as, but not limited to, surgery or radiation treatment. Where the combination therapy further comprises radiation treatment, the radiation treatment may be conducted at any suitable time so long as a beneficial effect from the co-action of the combination of the therapeutic agents and radiation treatment is achieved.

In some embodiments, the methods described herein relate to treating a subject having or diagnosed as having cancer. Subjects having cancer can be identified by a physician using current methods of diagnosing cancer. Symptoms and/or complications of cancer, e.g. diffuse large B-cell lymphoma, which characterize these conditions and aid in diagnosis are well known in the art and include but are not limited to, swollen lymph nodes, abdominal swelling and discomfort, weight loss, fever, or night sweats. Tests that may aid in a diagnosis of, e.g. diffuse large B-cell lymphoma include, but are not limited to, CT scan, CT/PET scan, endoscopic ultrasound, tissue biopsy, or bone marrow biopsy. A family history of cancer or exposure to risk factors for cancer (e.g. in the case of diffuse large B-cell lymphoma) can also aid in determining if a subject is likely to have cancer or in making a diagnosis of cancer.

In some embodiments, the methods described herein comprises a first step of selecting a subject having been diagnosed with cancer and/or having at least one mutation in an epigenetic selected from the group consisting of EZH2, KMT2D, CREBPP, and EP300. Subjects having cancer can be identified by a physician using current methods of diagnosing cancer. Symptoms and/or complications of cancer which characterize these conditions and aid in diagnosis are well known in the art and include but are not limited to, growth of a tumor, impaired function of the organ or tissue harboring cancer cells, etc. Tests that may aid in a diagnosis of, e.g. cancer include, but are not limited to, tissue biopsies and histological examination. A family history of cancer, or exposure to risk factors for cancer (e.g. tobacco products, radiation, etc.) can also aid in determining if a subject is likely to have cancer or in making a diagnosis of cancer.

In some embodiments of the various aspects disclosed herein, the method described herein comprises a step of assaying a sample from the subject for the following: (i) at least one mutation in an epigenetic modifier selected from the group consisting of EZH2, KMT2D, CREBPP, and EP300; (ii) over-expression of at least one Ikaros family member; (iii) over-expression of KDM4A and/or KDM4C: and/or (iv) at least one mutation in canonical Wnt signaling. Without limitations, the assaying step can include an assay selected from the group consisting of hybridization, sequencing, high-throughput sequencing, PCR, RT-qPCR, exome capture, allele-specific probe hybridization, allele-specific primer extension, allele-specific amplification, 5′ nuclease digestion, molecular beacon assay, oligonucleotide ligation assay, size analysis, single-stranded conformation polymorphism, immunohistochemistry, immunocytochemistry, flow cytometry, fluorescent-activated cell sorting (FACS), immunoblotting, radioimmunoassays, western blotting, immunoprecipitation, enzyme-linked immunosorbant assays (ELISA), lateral flow immunoassay test (LFIA), and any combinations thereof.

In some embodiments of the various aspects disclosed herein, the method comprises a step of assaying a sample from the subject for presence of at least one mutation in an epigenetic modifier selected from the group consisting of EZH2, KMT2D, CREBPP, and EP300, or a mutation in canonical Wnt signaling. Generally, a mutation will typically be present in the genomic DNA of a cell, e.g., a tumor or cancer cell. Accordingly, the mutation can be detected in either or both of the genomic DNA or the mRNA transcripts. The mutation can also be detected in the protein/polypeptide encoded/produced by the genomic DNA or the mRNA transcript.

Methods of detecting mutations in nucleic acids are well known in the art. For example, the presence of a mutation can be detected by determining the sequence of a genomic locus and/or an mRNA transcript Such molecules can be isolated, derived, or amplified from a biological sample, such as a tumor sample Nucleic acid (e.g. DNA) and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials, and proteinase K extraction can be used to obtain nucleic acid from blood (Roiff. A et al. PCR: Clinical Diagnostics and Research, Springer (1994)).

In some embodiments of the various aspects disclosed herein, the nucleic acid sequence of the epigenetic modifier, e.g., EZH2, KMT2D, CREBPP, and/or EP300, in a sample obtained from a subject can be determined and compared to a reference sequence to determine if a mutation is present in the subject. In some embodiments of the various aspects disclosed herein, the sequence of the target gene can be determined by sequencing the target gene (e.g. the genomic sequence and/or the mRNA transcript thereof). Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing, dideoxy chain termination, high-throughput sequencing, next generation sequencing, 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (3 ed.), Cold Spring Harbor Laboratory Press, Cold Spring Harbor, N Y, USA (2001); which are incorporated by reference herein in their entireties.

In some embodiments of the various aspects disclosed herein, sequencing can comprise exome sequencing (i.e. targeted exome capture). Exome sequencing comprises enriching for an exome(s) of interest and then sequencing the nucleic acids comprised by the enriched sample. Sequencing can be according to any method known in the art, e.g. those described above herein. Methods of enrichment can include, e.g. PCR, molecular inversion probes, hybrid capture, and in solution capture. Exome capture methodologies are well known in the art, see, e.g. Sulonen et al. Genome Biology 2011 12:R94; and Teer and Mullikin. Hum Mol Genet 2010 19:R2; which are incorporated by reference herein in their entireties. Kits for performing exome capture are available commercially, e.g. the TRIUSEQ™ Exome Enrichment Kit (Cat. No. FC-121-1008; Illumnia, San Diego, Calif.). Exome capture methods can also readily be adapted by one of skill in the art to enrich specific exomes of interest.

In some embodiments of the various aspects disclosed herein, the presence of a mutation can be determined using a probe that is specific for the sensitizing mutation. In some embodiments of the various aspects disclosed herein, the probe can be detectably labeled. In some embodiments of the various aspects disclosed herein, a detectable signal can be generated by the probe when a sensitizing mutation is present.

In some embodiments of the various aspects disclosed herein, the probe specific for the mutation can be a probe in a hybridization assay, i.e. the probe can specifically hybridize to a nucleic acid comprising a mutation (as opposed to a wild-type nucleic acid sequence) and the hybridization can be detected, e.g. by having the probe and or the target nucleic acid be detectably labeled. Hybridization assays are well known in the art and include, e.g. northern blots and Southern blots.

In some embodiments of the various aspects disclosed herein, the probe specific for the mutation can be a probe in a PCR assay, i.e. a primer. In general, the PCR procedure describes a method of gene amplification which is comprised of (i) sequence-specific hybridization of primers to specific genes within a nucleic acid sample or library, (ii) subsequent amplification involving multiple rounds of annealing, elongation, and denaturation using a thermostable DNA polymerase, and optionally, (iii) screening the PCR products for a band or product of the correct size. The primers used are oligonucleotides of sufficient length and appropriate sequence to provide initiation of polymerization, i.e. each primer is specifically designed to be complementary to a strand of the genomic locus to be amplified. In an alternative embodiment, the presence of a sensitizing mutation in an mRNA transcript can be determined by reverse-transcription (RT) PCR and by quantitative RT-PCR (QRT-PCR) or real-time PCR methods. Methods of RT-PCR and QRT-PCR are well known in the art. In some embodiments of the various aspects disclosed herein, the PCR product can be labeled, e.g. the primers can comprise a detectable label, or a label can be incorporated and/or bound to the PCR product, e.g. EtBr detection methods. Other non-limiting detection methods can include the detection of a product by mass spectroscopy or MALDI-TOF.

The term “label” refers to a composition capable of producing a detectable signal indicative of the presence of a reagent (e.g. a bound antibody reagent). Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

Detection of polypeptides comprising a mutation can be according to any method known in the art (e.g. mass spectroscopy, flow cytometry, and/or immunological-based methods) Immunological methods to detect polypeptides comprising a sensitizing mutation in accordance with the present technology include, but are not limited to antibody techniques such as immunohistochemistry, immunocytochemistry, flow cytometry, fluorescent-activated cell sorting (FACS), immunoblotting, radioimmunoassays, western blotting, immunoprecipitation, enzyme-linked immunosorbant assays (ELISA), and derivative techniques that make use of antibody reagents as described herein.

Immunochemical methods require the use of an antibody reagent specific for the target molecule (e.g. the antigen or in the embodiments described herein, a polypeptide or fragment thereof comprising a sensitizing mutation). In some embodiments of the various aspects disclosed herein, an antibody reagent for determining the presence of a mutation in a sample can be an antibody reagent specific for a polypeptide comprising a mutation, e.g. a mutation of KDM4A.

In some embodiments of the various aspects disclosed herein, the assays, methods, and/or systems described herein can comprise: an antibody reagent. In some embodiments of the various aspects disclosed herein, the antibody reagents and methods of using them described herein can comprise detecting the localization of an Ikaros family member, KDM4A and/or KDM4C, e.g. the relative concentrations of an Ikaros family member. KDM4A and/or KDM4C in the cytoplasm or nucleus. In some embodiments of the various aspects disclosed herein, the antibody reagent can be detectably labeled. In some embodiments of the various aspects disclosed herein, the antibody reagent can be attached to a solid support (e.g. bound to a solid support) In some embodiments of the various aspects disclosed herein, the solid support can comprise a particle (including, but not limited to an agarose or latex bead or particle or a magnetic particle), a bead, a nanoparticle, a polymer, a substrate, a slide, a coverslip, a plate, a dish, a well, a membrane, and/or a grating. The solid support can include many different materials including, but not limited to, polymers, plastics, resins, polysaccharides, silicon or silica based materials, carbon, metals, inorganic glasses, and membranes.

In one embodiment, an assay, method, and/or system as described herein can comprise an ELISA. In an exemplary embodiment, a first antibody reagent can be immobilized on a solid support (usually a polystyrene micro titer plate). The solid support can be contacted with a sample obtained from a subject, and the antibody reagent will bind (“capture”) antigens for which it is specific (e.g. a polypeptide comprising a sensitizing mutation). The solid support can then be contacted with a second labeled antibody reagent (e.g. a detection antibody reagent). The detection antibody reagent can, e.g. comprise a detectable signal, be covalently linked to an enzyme, or can itself be detected by a secondary antibody, which is linked to an enzyme through bio-conjugation. The presence of a signal indicates that both the first antibody reagent immobilized on the support and the second “detection” antibody reagent have bound to an antigen, i.e. the presence of a signal indicated the presence of polypeptide comprising a sensitizing mutation. Between each step the plate is typically washed with a mild detergent solution to remove any proteins or antibodies that are not specifically bound. After the final wash step the plate is developed by adding an enzymatic substrate to produce a visible signal, which indicates the presence of a sensitizing mutation in the sample Older ELISAs utilize chromogenic substrates, though newer assays employ fluorogenic substrates with much higher sensitivity. There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980: Campbell et al., “Methods and Immunology”, W. A. Benjamin, Inc, 1964; and Oellerich, M. 1984, J. Clin. Chem. Clin. Biochem. 22:895-904, contents of all of which are incorporated herein by reference in their entirety.

In some embodiments, the assays, systems, and methods described herein can comprise a lateral flow immunoassay test (LFIA), also known as the immunochromatographic assay, or strip test to measure or determine the presence of a polypeptide comprising a sensitizing mutation LFIAs are a simple device intended to detect the presence (or absence) of a target in a sample. There are currently many LFIA tests are used for medical diagnostics either for home testing, point of care testing, or laboratory use. LFIA tests are a form of immunoassay in which the test sample flows along a solid substrate via capillary action. After the sample is applied to the test it encounters a colored antibody reagent, which mixes with the sample, and if bound to a portion of the sample, transits the substrate encountering lines or zones which have been pretreated with a second antibody reagent. Depending upon the presence or absence of the target in the sample the colored antibody reagent can become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, tumor cell lysates etc. Strip tests are also known as dip stick test, the name bearing from the literal action of “dipping” the test strip into a fluid sample to be tested. LFIA strip test are easy to use, require minimum training and can easily be included as components of point-of-care test (POCT) diagnostics to be use on site in the field. LFIA tests can be operated as either competitive or sandwich assays. Sandwich LFIAs are similar to sandwich ELISA. The sample first encounters colored particles, which are labeled with antibody reagents specific for a target. The test line will also contain antibody reagents. The test line will show as a colored band in positive samples. In some embodiments of the various aspects disclosed herein, the lateral flow immunoassay can be a double antibody sandwich assay, a competitive assay, a quantitative assay or variations thereof. There are a number of variations on lateral flow technology. It is also possible to apply multiple capture zones to create a multiplex test.

A typical test strip consists of the following components: (1) sample application area comprising an absorbent pad (i. e. the matrix or material) onto which the test sample is applied; (2) conjugate or reagent pad—this contains antibody reagent(s) specific to the target which can be conjugated to colored particles (usually colloidal gold particles, or latex microspheres); (3) test results area comprising a reaction membrane—typically a hydrophobic nitrocellulose or cellulose acetate membrane onto which antibody reagents are immobilized in a line across the membrane as a capture zone or test line (a control zone may also be present, containing antibodies specific for the antibody reagents conjugated to the particles or microspheres): and (4) optional wick or waste reservoir—a further absorbent pad designed to draw the sample across the reaction membrane by capillary action and collect it. The components of the strip are usually fixed to an inert backing material and may be presented in a simple dipstick format or within a plastic casing with a sample port and reaction window showing the capture and control zones. While not strictly necessary, most tests will incorporate a second line, which contains an antibody that picks up free latex/gold in order to confirm the test has operated correctly.

The use of “dip sticks” or LFIA test strips and other solid supports have been described in the art in the context of an immunoassay for a number of antigen biomarkers. U.S. Pat. Nos. 4,943,522; 6,485,982; 6,187,598; 5,770,460; 5,622,871, 6,565,808. U.S. patent application Ser. No. 10/278,676; U.S. Ser. No. 09/579,673 and U.S. Ser. No. 10/717,082, which are incorporated herein by reference in their entirety, are non-limiting examples of such lateral flow test devices. Three U.S. patents (U.S. Pat. No. 4,444,880, issued to H. Tom. U.S. Pat. No. 4,305,924, issued to R. N. Piasio; and U.S. Pat. No. 4,135,884, issued to J. T. Shen) describe the use of “dip stick” technology to detect soluble antigens via immunochemical assays. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teaching of these “dip stick” technology for the detection of a sensitizing mutation.

Immunochemistry is a family of techniques based on the use of a specific antibody, wherein antibodies are used to specifically target molecules inside or on the surface of cells. In some embodiments of the various aspects disclosed herein, immunohistochemistry (“IHC”) and immunocytochemistry (“ICC”) techniques can be used to detect the presence of a sensitizing mutation IHC is the application of immunochemistry to tissue sections, whereas ICC is the application of immunochemistry to cells or tissue imprints after they have undergone specific cytological preparations such as, for example, liquid-based preparations. In some instances, signal amplification may be integrated into the particular protocol, wherein a secondary antibody, that includes a label, follows the application of an antibody reagent specific for a polypeptide comprising a sensitizing mutation Typically, for immunohistochemistry, tissue obtained from a subject and fixed by a suitable fixing agent such as alcohol, acetone, and paraformaldehyde, is sectioned and reacted with an antibody. Conventional methods for immunohistochemistry are described in Buchwalow and Bocker (Eds) “Immunohistochemistry. Basics and Methods” Springer (2010): Lin and Prichard “Handbook of Practical Immunohistochemistry” Springer (2011); which are incorporated by reference herein in their entireties. In some embodiments of the various aspects disclosed herein, immunocytochemistry may be utilized where, in general, tissue or cells are obtained from a subject are fixed by a suitable fixing agent such as alcohol, acetone, and paraformaldehyde, to which is reacted an antibody. Methods of immunocytological staining of human samples is known to those of skill in the art and described, for example, in Burry. “Immunocytochemistry. A Practical Guide for Biomedical Research” Springer (2009); which is incorporated by reference herein in its entirety.

In some embodiments of the various aspects disclosed herein, one or more of the antibody reagents described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into an antibody reagent are well known in the art.

In some embodiments of the various aspects disclosed herein, detectable labels can include labels that can be detected by spectroscopic, photochemical, biochemical, immunochemical, electromagnetic, radiochemical, or chemical means, such as fluorescence, chemifluorescence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies) The detectable label can be linked by covalent or non-covalent means to the antibody reagent. Alternatively, a detectable label can be linked such as by directly labeling a molecule that achieves binding to the antibody reagent via a ligand-receptor binding pair arrangement or other such specific recognition molecules Detectable labels can include, but are not limited to radioisotopes, bioluminescent compounds, chromophores, antibodies, chemiluminescent compounds, fluorescent compounds, metal chelates, and enzymes.

In other embodiments, the detection antibody is label with a fluorescent compound. When the fluorescently labeled antibody is exposed to light of the proper wavelength, its presence can then be detected due to fluorescence. In some embodiments of the various aspects disclosed herein, a detectable label can be a fluorescent dye molecule, or fluorophore including, but not limited to fluorescein, phycoerythrin, phycocyanin, o-phthaldehyde, fluorescamine. Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfluorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfluorescein (JOE or J), N,N,N′,N′-tetramethyl-6carboxyrhodamine (TAMRA or T), 6-carboxy-X-rhodamine (ROX or R), S-carboxyrhodamine-6G (R6G5 or G5), 6-carboxyrhodamine-6G (R6G6 or G6), and rhodamine 110, cyanine dyes, e.g. Cy3, Cy5 and Cy7 dyes; coumarins, e.g. umbelliferone; benzimide dyes, e.g. Hoechst 33258, phenanthridine dyes, e.g. Texas Red, ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. In some embodiments of the various aspects disclosed herein, a detectable label can be a radiolabel including, but not limited to ³H, ¹²⁵I, ³⁵S, ¹⁴C, ³²P, and ³³P. In some embodiments of the various aspects disclosed herein, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments of the various aspects disclosed herein, a detectable label is a chemiluminescent label, including, but not limited to lucigenin, luminol, luciferin, isoluminol, theromatic acridinium ester, imidazole, acridinium salt and oxalate ester. In some embodiments of the various aspects disclosed herein, a detectable label can be a spectral colorimetric label including, but not limited to colloidal gold or colored glass or plastic (e.g., polystyrene, polypropylene, and latex) beads.

In some embodiments of the various aspects disclosed herein, antibodies can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e. g. from DAKO; Carpinteria, Calif. An antibody reagent can also be detectably labeled using fluorescence emitting metals such as ¹⁵²Eu, or others of the lanthanide series. These metals can be attached to the antibody reagent using such metal chelating groups as diethylenetriaminepentaacetic acid (DTPA) or ethylenediaminetetraacetic acid (EDTA).

In some embodiments of the various aspects disclosed herein, the sequence, level, activity, and/or localization can be compared to a reference sample or level. In some embodiments of the various aspects disclosed herein, the reference level can be the level in a healthy subject not diagnosed as having or not having cancer. In some embodiments of the various aspects disclosed herein, the reference level can be the level in a healthy, non-cancerous cell from the same subject.

The term “sample” or “test sample” as used herein denotes a sample taken or isolated from a biological organism, e.g., a tumor sample from a subject. Exemplary biological samples include, but are not limited to, a biofluid sample; serum; plasma; urine; saliva; a tumor sample; a tumor biopsy and/or tissue sample etc. The term also includes a mixture of the above-mentioned samples. The term “test sample” also includes untreated or pretreated (or pre-processed) biological samples. In some embodiments of the various aspects disclosed herein, a test sample can comprise cells from subject. In some embodiments of the various aspects disclosed herein, a test sample can be a tumor cell test sample, e.g. the sample can comprise cancerous cells, cells from a tumor, and/or a tumor biopsy.

The test sample can be obtained by removing a sample of cells from a subject, but can also be accomplished by using previously isolated cells (e.g. isolated at a prior timepoint and isolated by the same or another person). In addition, the test sample can be freshly collected or a previously collected sample.

In some embodiments of the various aspects disclosed herein, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments of the various aspects disclosed herein, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments of the various aspects disclosed herein, the test sample is a clarified test sample, for example, by centrifugation and collection of a supernatant comprising the clarified test sample. In some embodiments of the various aspects disclosed herein, a test sample can be a pre-processed test sample, for example, supernatant or filtrate resulting from a treatment selected from the group consisting of centrifugation, filtration, thawing, purification, and any combinations thereof. In some embodiments of the various aspects disclosed herein, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the presence of a sensitizing mutation as described herein.

In some embodiments of the various aspects disclosed herein, the method can further comprise a step of obtaining a test sample from a subject. In some embodiments of the various aspects disclosed herein, the subject can be a human subject.

It is noted that obtaining the test sample, detecting the mutation and administering of the compound can be done by a single person or different persons.

In some embodiments, provided herein is an assay comprising: contacting a tumor cell sample obtained from a subject having cancer with a nucleic acid probe to detect the presence of a mutation in EZH2, KTM2D, CREBBP and/or EP300; and detecting the presence or intensity of a signal which indicates the presence of a mutation in EZH2, KTM2D, CREBBP and/or EP300; wherein the presence of a mutation of EZH2, KTM2D, CREBBP and EP300 indicates the subject has a cancer which will respond to a treatment with a compound targeting KDM4 or KDM5.

In one aspect, provided herein is a method of classifying a cancer as an KDM4 or KDM5 combination treatment responsive cancer, the method comprising: detecting a mutation of EZH2, KTM2D, CREBBP, or EP300 in a tumor cell sample; wherein the presence of a mutation of EZH2, KTM2D, CREBBP, or EP300 indicates the cancer is a KDM4 or KDM5 combination treatment responsive cancer. As used herein, the term “KDM4 or KDM5 combination treatment responsive cancer” refers to a cancer that, after being contacted with an effective dose of compound targeting KDM4 or KDM5 will be more sensitive to at least one chemotherapeutic agent than the same cancer would be in the absence of being contacted with the compound targeting KDM4 or KDM5.

Data presented herein show that the exemplary compound targeting a KDM4/KDM5 family member does not induce apoptosis in all cancer cells. Instead, inventors have discovered inter alia that sensitivity of cancer cells treatments that target KDM4/KDM5 family members is dependent on whether the cancer cell contains one or more of the following: (i) at least one mutation in an epigenetic modifier selected from the group consisting of EZH2, KMT2D, CREBPP, and EP300; (ii) over-expression of at least one Ikaros family member; (iii) over-expression of KDM4A and/or KDM4C; and/or (iv) at least one mutation in canonical Wnt signaling. Accordingly, cancer cells that can be treated by methods and compositions of the invention include cells where the cell comprises: (i) at least one mutation in an epigenetic modifier selected from the group consisting of EZH2, KMT2D, CREBPP, and EP300; (ii) over-expression of at least one Ikaros family member; (iii) over-expression of KDM4A and/or KDM4C; and/or (iv) at least one mutation in canonical Wnt signaling. Exemplary cells can be cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus.

Further, cancers can be treated by methods and compositions of the invention include cancers where the subject has one of the following: (i) at least one mutation in an epigenetic modifier selected from the group consisting of EZH2, KMT2D, CREBPP, and EP300; (ii) over-expression of at least one Ikaros family member; (iii) over-expression of KDM4A and/or KDM4C; and/or (iv) at least one mutation in canonical Wnt signaling. Exemplary cancers include, but are not limited to, neoplasm, malignant: carcinoma; carcinoma, undifferentiated; giant and spindle cell carcinoma; small cell carcinoma, papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; bronchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma, papillary and follicular adenocarcinoma; nonencapsulating sclerosing carcinoma; adrenal cortical carcinoma; endometroid carcinoma, skin appendage carcinoma: apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; leydig cell tumor, malignant; lipid cell tumor, malignant; paraganglioma, malignant; extra-mammary paraganglioma, malignant; pheochromocytoma; glomangiosarcoma: malignant melanoma; amelanotic melanoma; superficial spreading melanoma; malig melanoma in giant pigmented nevus: epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant: brenner tumor, malignant; phyllodes tumor, malignant; synovial sarcoma; mesothelioma, malignant, dysgerminoma, embryonal carcinoma; teratoma, malignant; strumaovarii, malignant: choriocarcinoma, mesonephroma, malignant; hemangiosarcoma: hemangioendothelhoma, malignant: Kaposi's sarcoma: hemangiopericytoma, malignant; lymphangiosarcoma; osteosarcoma; juxtacortical osteosarcoma: chondrosarcoma; chondroblastoma, malignant; mesenchymal chondrosarcoma: giant cell tumor of bone; Ewing's sarcoma, odontogenic tumor, malignant; ameloblasticodontosarcoma; ameloblastoma, malignant; ameloblasticfibrosarcoma; pinealoma, malignant, chordoma; glioma, malignant; ependymoma; astrocytoma; protoplasmic astrocytoma; fibrillary astrocytoma; astroblastoma; glioblastoma; oligodendroghlioma; oligodendroblastoma; primitive neuroectodermal; cerebellar sarcoma; ganglioneuroblastoma, neuroblastoma; retinoblastoma; olfactory neurogenic tumor; meningioma, malignant; neurofibrosarcoma; neurilemmoma, malignant; granular cell tumor, malignant; malignant lymphoma, Hodgkin's disease, hodgkin's, paragranuloma, malignant lymphoma, small lymphocytic; malignant lymphoma, large cell, diffuse; malignant lymphoma, follicular; mycosis fungoides, other specified non-Hodgkin's lymphomas: malignant histiocytosis; multiple myeloma; mast cell sarcoma; immunoproliferative small intestinal disease; leukemia; lymphoid leukemia; plasma cell leukemia, erythroleukemia; lymphosarcomacell leukemia, myeloid leukemia; basophilic leukemia; eosinophilic leukemia; monocytic leukemia; mast cell leukemia; megakaryoblastic leukemia; myeloid sarcoma; and hairy cell leukemia.

For example, cancer can be adrenal cancer, acute lymphoblastic leukemia, acute myelogenous leukemia, astrocytoma, basal cell carcinoma, bile duct cancer, bladder cancer, bone cancer, breast cancer, brain cancer, carcinoma, cardiac tumor, cervical cancer, childhood cancers, chronic lymphocytic leukemia, chronic myelogenous leukemia, colorectal cancer, embryonal tumor, epithelial cancer, esophageal cancer, gastrointestinal cancer, germ cell tumor, gallbladder cancer, gastric cancer, glioma, head and neck cancer, hematological malignancy, Hodgkin's lymphoma, non-Hodgkin's lymphoma, intestinal cancer, intraocular melanoma, kidney cancer, laryngeal cancer, leukemia, lung cancer, liver cancer, malignant peripheral nerve sheath tumor, melanoma, mesothelioma, nasopharyngeal carcinoma, neuroblastoma, neurofibroma, oral cancer, non-small cell lung cancer, osteosarcoma, ovarian cancer, pituitary tumor, prostate cancer, pancreatic cancer, retinoblastoma, rhabdomyosarcoma, sarcoma, small cell lung cancer, testicular cancer, throat cancer, thyroid cancer, transitional cell carcinoma, urogenital cancer, urothelial carcinoma, uterine cancer, vaginal cancer, or Wilms' tumor.

In some embodiments of any of the aspects described herein, the cancer can be diffuse large B-cell lymphoma; pancreatic cancer; pancreatic ductal adenocarcinoma; metastatic breast cancer; breast cancer; bladder cancer; small cell lung cancer; lung cancer; ovarian cancer; stomach cancer; uterine cancer; mesothelioma; adenoid cystic carcinoma; lymphoid neoplasm; kidney cancer; colorectal cancer; adenoid cystic carcinoma; prostate cancer; cervical cancer; head and neck cancer; or glioblastoma. In some embodiments of any of the aspects described herein, the cancer is diffuse large B-cell lymphoma, colorectal cancer, or lung cancer.

In some embodiments, the cancer is diffuse large B-cell lymphoma. Diffuse large B-cell lymphoma is the most common type of non-Hodgkin lymphoma. Genetic analyses have revealed molecular heterogeneity of DLBCL tumors, which has led to the cell-of-origin (COO) classification of DLBCL into two subtypes: germinal center B-cell-like (GCB) and activated B-cell-like (ABC). Accordingly, in some embodiments of the various aspects disclosed herein, DLBCL is germinal center B-cell-like DLBCL. In some other embodiments of the various aspects disclosed herein, DLBCL is activated B-cell-like DLBCL.

### Dosage and Administration

A variety of means for administering the compounds and compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The dosage range depends upon the potency, and includes amounts large enough to produce the desired effect, e.g., a decrease in tumor size. The dosage should not be so large as to cause unacceptable adverse side effects. Generally, the dosage will vary with the type of agent (e.g., an antibody or fragment, small molecule, siRNA, etc.), and with the age, condition, and sex of the patient. The dosage can be determined by one of skill in the art and can also be adjusted by the individual physician in the event of any complication. Typically, the dosage will range from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL.

Administration of the doses recited above can be repeated for a limited period of time or as necessary. In some embodiments, the doses are given once a day, or multiple times a day, for example but not limited to three times a day. In some embodiments, the doses recited above are administered daily for several weeks or months. The duration of treatment depends upon the subject's clinical progress and responsiveness to therapy. Continuous, relatively low maintenance doses are contemplated after an initial higher therapeutic dose.

A therapeutically effective amount is an amount of an agent that is sufficient to produce a statistically significant, measurable change in e.g., tumor size, tumor volume, tumor growth rate, etc. (see “Efficacy Measurement” below). Such effective amounts can be gauged in clinical trials as well as animal studies for a given inhibitor.

Agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. For the treatment of tumors, the agent can be administered systemically, or alternatively, can be administered directly to the tumor e.g., by intratumor injection or by injection into the tumor's primary blood supply.

Therapeutic compositions containing the compound targeting KDM4/KDM5 can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

### Efficacy Measurement

The efficacy of a given treatment for cancer, such as DLBCL can be determined by the skilled clinician. However, a treatment is considered “effective treatment,” as the term is used herein, if any one or all of the signs or symptoms of, as but one example, cancer are altered in a beneficial manner, other clinically accepted symptoms or markers of disease are improved or ameliorated, e.g., by at least 10% following treatment with an inhibitor. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the pathogenic growth of cancer cells; or (2) relieving the disease, e.g., causing regression of symptoms, reducing the size of a tumor; and (3) preventing or reducing the likelihood of the development of a castration-resistant cancer or a metastatic disease thereof.

An effective amount for the treatment of cancer means that amount which, when administered to a mammal in need thereof, is sufficient to result in effective treatment as that term is defined herein, for that disease. Efficacy of an agent can be determined by assessing physical indicators of cancer, such as e.g., tumor size, tumor volume, tumor growth rate, metastatic phenotype, etc.

The term “effective amount” as used herein refers to the amount of a composition (e.g. JIB04) needed to alleviate at least one or more symptom of the disease or disorder, and relates to a sufficient amount of pharmacological composition to provide the desired effect. The term “therapeutically effective amount” therefore refers to an amount of a composition that is sufficient to provide a particular anti-tumor effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active ingredient, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., tumor growth, among others. The dosage can be determined by a physician and adjusted, as necessary, to suit observed effects of the treatment.

For administering to a subject, the compound targeting a KDM4/KDM5 family member can be formulated in a pharmaceutical composition comprising the compound and optionally a pharmaceutically acceptable carrier. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, as described herein.

In some embodiments, the compound targeting a KDM4/KDM5 family member can be co-formulated with at least one of a CDK inhibitor, a BCR inhibitor and Pyrivinium, in a pharmaceutical composition optionally comprising a pharmaceutically acceptable carrier. For example, the compound targeting a KDM4/KDM5 family member can be co-formulated with a CDK inhibitor, such as a Cdk7 inhibitor, e.g, THZ-1, or the compound targeting a KDM4/KDM5 family member can be co-formulated with a BCR inhibitor, such as a BTK inhibitor, e.g., Ibrutinib, or the compound targeting a KDM4/KDM5 family member can be co-formulated with Pyrivinium.

When the compound targeting a KDM4/KDM5 family member can be co-formulated with at least one of a CDK inhibitor, a BCR inhibitor and Pyrivinium, amount of at least one of the KDM4/KDM5 family member, CDK inhibitor, BCR inhibitor and/or Pyrivinium in the composition can be such an amount that is ineffective for treating cancer when that component is administered in that amount.

In some embodiments of the various aspects described herein, the pharmaceutical composition can be a parenteral dose form. Since administration of parenteral dosage forms typically bypasses the patient's natural defenses against contaminants, parenteral dosage forms are preferably sterile or capable of being sterilized prior to administration to a patient Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms as disclosed within are well known to those skilled in the art Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a composition as disclosed herein can also be incorporated into the parenteral dosage forms of the disclosure, including conventional and controlled-release parenteral dosage forms.

Pharmaceutical compositions can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug, 5) reduction in local or systemic side effects; 6) minimization of drug accumulation, 7) reduction in blood level fluctuations; 8) improvement in efficacy of treatment; 9) reduction of potentiation or loss of drug activity; and 10) improvement in speed of control of diseases or conditions Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3.598,123; 4,008,719; 5,674,533; 5,059,595; 5,591,767; 5,120,548; 5,073,543; 5,639,476; 5,354,556; 5,733,566; and 6,365,185 BI: each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

### Definitions

As used herein, the term “epigenetic modifier” refers to a material that affects, is believed to affect, or tends to affect gene expression and function. Also as used herein, “epigenetically driven” may refer to any material that is affected by, or tended to be affected by, gene expression and function.

As used herein, the term “biological sample” refers to a fluid sample, a cell sample, a tissue sample, or an organ sample obtained from a subject or patient. For the purposes of isolating circulating tumor cells, the biological sample is typically a whole blood sample, but can also be a partially separated (e.g., centrifuged) blood sample provided that the biological sample comprises at least one circulating tumor cell, as that term is used herein. In some embodiments, while not necessary, a cell or population of cells, an exosome, a quantity of tissue or fluid are obtained from a subject to first detect the presence of prostate cancer prior to isolation of circulating tumor cells.

The term “sample” or “test sample” as used herein denotes a sample taken or isolated from a biological organism, e.g., a tumor sample from a subject. Exemplary biological samples include, but are not limited to, a biofluid sample; serum; plasma; urine; saliva; a tumor sample; a tumor biopsy and/or tissue sample etc. The term also includes a mixture of the above-mentioned samples. The term “test sample” also includes untreated or pretreated (or pre-processed) biological samples. In some embodiments, a test sample can comprise cells from subject. In some embodiments, a test sample can be a tumor cell test sample, e.g. the sample can comprise cancerous cells, cells from a tumor, and/or a tumor biopsy. Further, the term “sample” includes any material derived by processing such a sample. Derived samples can, for example, include nucleic acids or proteins extracted from the sample or obtained by subjecting the sample to techniques such as amplification or reverse transcription of mRNA, isolation and/or purification of certain components, etc.

As used herein, the term “inhibitor” refers to an agent which can decrease the expression and/or activity of the target, e.g. by at least 10% or more, e.g. by 10% or more, 50% or more, 70% or more, 80% or more, 90% or more, 95% or more, or 98% or more. The efficacy of an inhibitor of a particular target e.g. its ability to decrease the level and/or activity of the target can be determined, e.g. by measuring the level of an expression product and/or the activity of the target. Methods for measuring the level of a given mRNA and/or polypeptide are known to one of skill in the art, e.g. RT-PCR with primers can be used to determine the level of RNA and Western blotting with an antibody can be used to determine the level of a polypeptide. The activity of a target can be determined using methods known in the art, e.g. measuring the expression level of a genes regulated by KDM4 or KDM5 family members as described herein. In some embodiments, the inhibitor can be an inhibitory nucleic acid or a small molecule.

“Inhibitors”, “activators”, and “modulators” of KDM4A, KDM4B, KDM4C, KDM4D, KDM5A, KDM5B, KDM5C, or KDM5D polynucleotide and polypeptide sequences are used to refer to activating, inhibitory, or modulating molecules identified using in vitro and in vivo assays of KDM4A, KDM4B, KDM4C, KDM4D, KDM5A, KDM5B, KDM5C, or KDM5D polynucleotide and polypeptide sequences. Inhibitors are compounds that, e.g., bind to, partially or totally block activity, decrease, prevent, delay activation, inactivate, desensitize, or down regulate the activity or expression of KDM4A, KDM4B, KDM4C, KDM4D, KDM5A, KDM5B, KDM5C, or KDM5D proteins, e.g., antagonists. “Activators” are compounds that increase, open, activate, facilitate, enhance activation, sensitize, agonize, or up regulate KDM4A, KDM4B, KDM4C, KDM4D, KDM5A, KDM5B, KDM5C, or KDM5D protein activity, e.g., agonists. Inhibitors, activators, or modulators also include genetically modified versions of KDM4A, KDM4B, KDM4C, KDM4D, KDM5A, KDM5B, KDM5C, or KDM5D proteins, e.g., versions with altered activity, as well as naturally occurring and synthetic ligands, antagonists, agonists, antibodies, peptides, cyclic peptides, nucleic acids, siRNA molecules, antisense molecules, ribozymes, small chemical molecules and the like. Such assays for inhibitors and activators include, e.g., expressing KDM4A, KDM4B, KDM4C, KDM4D, KDM5A, KDM5B, KDM5C, or KDM5D protein in vitro, in cells, or cell membranes, applying putative modulator compounds, and then determining the functional effects on activity, as described above.

The term “statistically significant” or “significantly” refers to statistical significance and generally means a two standard deviation (2SD) above normal, or higher. The term refers to statistical evidence that there is a difference. It is defined as the probability of making a decision to reject the null hypothesis when the null hypothesis is actually true. The decision is often made using the p-value.

As used herein, the terms “chemotherapy,” “anti-cancer agent,” or “chemotherapeutic agent” refer to any chemical agent with therapeutic usefulness in the treatment of diseases characterized by abnormal cell growth, and particularly cell growth associated with DLBCL. Such diseases include tumors, neoplasms and cancer as well as diseases characterized by hyperplastic growth. An anti-cancer agent or chemotherapeutic agent differs from a hormonal therapy, as the term is used herein, in that an anti-cancer or chemotherapeutic agent does not directly target AR pathways. Typically, in the context of the present disclosure, such chemotherapy agents are considered to be second- or third-line therapies that are applied following failure of a subject to adequately respond to first-line hormonal therapies for treatment of prostate cancer, or more frequently following the emergence of a hormone-resistant phenotype in an individual in which hormonal therapy was initially effective in reducing tumor load. Chemotherapeutic agents as used herein encompass both chemical and biological agents. These agents function to inhibit a cellular activity upon which the cancer cell depends for continued survival. Categories of chemotherapeutic agents include alkylating/alkaloid agents, antimetabolites, and miscellaneous antineoplastic drugs. Most if not all of these agents are directly toxic to cancer cells and do not require immune stimulation. In one embodiment, a chemotherapeutic agent is a radioactive molecule. One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., ©2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993). In one embodiment, the chemotherapeutic agent comprises a taxane chemotherapeutic agent. For example, in one embodiment, the chemotherapeutic agent is docetaxol.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with a malignant condition or cancer. Treatment is generally “effective” if one or more symptoms or clinical markers are reduced. Alternatively, treatment is “effective” if the progression of a disease is reduced or halted. That is, “treatment” includes not just the improvement of symptoms or markers, but can also include a cessation or at least slowing of progress or worsening of symptoms that would be expected in absence of treatment. Beneficial or desired clinical results include, but are not limited to, alleviation of one or more symptom(s) of a malignant disease, diminishment of extent of a malignant disease, stabilized (i.e., not worsening) state of a malignant disease, delay or slowing of progression of a malignant disease, amelioration or palliation of the malignant disease state, and remission (whether partial or total), whether detectable or undetectable. The term “treatment” of a disease also includes providing relief from the symptoms or side-effects of the disease (including palliative treatment).

As used herein, “alleviating a symptom of a cancer” is ameliorating any condition or symptom associated with the cancer. As compared with an equivalent untreated control, such reduction is by at least 5%, 10%, 20%, 40%, 50%, 60%, 80%, 90%, 95%, 99% or more as measured by any standard technique.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of cancer. A subject can be male or female.

A subject can be one who has been previously diagnosed with or identified as suffering from or having a condition in need of treatment (e.g. cancer) or one or more complications related to such a condition, and optionally, have already undergone treatment for cancer or the one or more complications related to cancer. Alternatively, a subject can also be one who has not been previously diagnosed as having cancer or one or more complications related to cancer. For example, a subject can be one who exhibits one or more risk factors for cancer or one or more complications related to cancer or a subject who does not exhibit risk factors.

A “subject in need” of treatment for a particular condition can be a subject having that condition, diagnosed as having that condition, or at risk of developing that condition.

As used herein “an increased likelihood” refers to at least a 1.5-fold greater likelihood of a particular scenario, e.g. a 1.5 fold, or 2-fold, or 2.5-fold, or 3-fold, or 4-fold, or greater risk. As used herein “a decreased likelihood” refers to at least a 80% lower likelihood of a particular scenario, e.g. a 80% lower, a 50% lower, 40% lower, 30% lower, 20% lower, 10% lower, or lower risk.

As used herein, the term “cancer” or “tumor” refers to an uncontrolled growth of cells which interferes with the normal functioning of the bodily organs and systems. A subject that has a cancer or a tumor is a subject having objectively measurable cancer cells present in the subject's body. Included in this definition are benign and malignant cancers, as well as dormant tumors or micrometastases. Cancers which migrate from their original location and seed vital organs can eventually lead to the death of the subject through the functional deterioration of the affected organs.

As used herein “gene copy number” refers to the number of copies of a given gene that occur in the genome. As used herein, “gene amplification” refers to the presence of a greater than normal gene copy number within the cell. In some embodiments, the copies are located on the same chromosome. In some embodiments, the copies are located on more than one chromosome. In some embodiments, gene copy number can include partial copies of a gene, e.g. less than the full coding sequence.

As used herein, “expression level” refers to the number of mRNA molecules and/or polypeptide molecules encoded by a given gene that are present in a cell or sample. Expression levels can be increased or decreased relative to a reference level.

The term “agent” refers generally to any entity which is normally not present or not present at the levels being administered to a cell, tissue or subject. An agent can be selected from a group including but not limited to: polynucleotides; polypeptides; small molecules; and antibodies or antigen-binding fragments thereof. A polynucleotide can be RNA or DNA, and can be single or double stranded, and can be selected from a group including, for example, nucleic acids and nucleic acid analogues that encode a polypeptide. A polypeptide can be, but is not limited to, a naturally-occurring polypeptide, a mutated polypeptide or a fragment thereof that retains the function of interest. Further examples of agents include, but are not limited to a nucleic acid aptamer, peptide-nucleic acid (PNA), locked nucleic acid (LNA), small organic or inorganic molecules; saccharide; oligosaccharides; polysaccharides; biological macromolecules, peptidomimetics; nucleic acid analogs and derivatives; extracts made from biological materials such as bacteria, plants, fungi, or mammalian cells or tissues and naturally occurring or synthetic compositions. An agent can be applied to the media, where it contacts the cell and induces its effects. Alternatively, an agent can be intracellular as a result of introduction of a nucleic acid sequence encoding the agent into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety selected, for example, from unsubstituted or substituted alkyl, aromatic, or heterocyclyl moieties including macrolides, leptomycins and related natural products or analogues thereof. Agents can be known to have a desired activity and/or property, or can be selected from a library of diverse compounds. As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight more than about 50, but less than about 5000 Daltons (5 kD). Preferably the small molecule has a molecular weight of less than 3 kD, still more preferably less than 2 kD, and most preferably less than 1 kD. In some cases it is preferred that a small molecule have a molecular mass equal to or less than 700 Daltons.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein an “antibody” refers to IgG, IgM, IgA, IgD or IgE molecules or antigen-specific antibody fragments thereof (including, but not limited to, a Fab, F(ab′)2, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As used herein, the term “therapeutically effective amount” means that amount necessary, at least partly, to attain the desired effect, or to delay the onset of, inhibit the progression of, or halt altogether, the onset or progression of the particular disease or disorder being treated (e.g., castration-resistant prostate cancer). Such amounts will depend, of course, on the particular condition being treated, the severity of the condition and individual patient parameters including age, physical condition, size, weight and concurrent treatment. These factors are well known to those of ordinary skill in the art and can be addressed with no more than routine experimentation. In some embodiments, a maximum dose of the anti-cancer agent is used, that is, the highest safe dose according to sound medical judgment. It will be understood by those of ordinary skill in the art, however, that a lower dose or tolerable dose can be administered for medical reasons, psychological reasons or for virtually any other reason.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference value or reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level or non-detectable level as compared to a reference sample), or any decrease between 10-100% as compared to a reference level.

The terms “increased”, “increase” or “enhance” or “activate” are all used herein to generally mean an increase by a statistically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

As used herein the term “comprising” or “comprises” is used in reference to compositions, methods, and respective component(s) thereof, that are essential to the claimed invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the claimed invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); Current Protocols in Cell Biology (CPCB) (Juan S. Bonifacino et. al. ed., John Wiley and Sons, Inc.), and Culture of Animal Cells: A Manual of Basic Technique by R Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Some embodiments of the various aspects disclosed herein can be defined according to any of the following numbered paragraphs:
1) A method for treating cancer, the method comprising: administering a therapeutically effective amount of a compound targeting a KDM4 or KDM5 family member to a subject in need thereof, wherein the subject has at least one mutation in an epigenetic modifier selected from the group consisting of: EZH2, KMT2D, CREBPP, and EP300.
2) The method of paragraph 1, wherein the compound is an inhibitor of a KDM4 family member.
3) The method of paragraph 1 or 2, wherein the compound is an inhibitor of KDM4A, KDM4B or KDM4C.
4) The method of any one of paragraphs 1-3, wherein the compound is a KDM4C inhibitor.
5) The method of any one of paragraphs 1-4, wherein the compound is 5-Chloro-2-[(E)-2-[phenyl(pyridin-2-yl)methylidene]hydrazin-1-yl]pyridine (JIB04).
6) The method of paragraph 1, wherein the compound is an inhibitor of KDM5A or KDM5B.
7) The method of paragraph 1, wherein the compound is an activator of KDM5C.
8) A method for treating cancer, the method comprising: administering a therapeutically effective amount of JIB04 to a subject in need thereof, wherein the subject has at least one mutation in an epigenetic modifier selected from the group consisting of: EZH2, KMT2D, CREBPP, and EP300.
9) The method of any one of paragraphs 1-8, wherein the cancer is selected from the group consisting of: diffuse large B-cell lymphoma (DLBCL), colorectal cancer, and lung cancer.
10) The method of any one of paragraphs 1-9, wherein the cancer is diffuse large B-cell lymphoma.
11) The method of paragraphs 8 and 9, wherein the cancer results from increased activation of canonical WNT signaling.
12) The method of any one of paragraphs 1-11, further comprising a first step of selecting a subject having at least one mutation in an epigenetic modifier selected from the group consisting of: EZH2, KMT2D, CREBPP, and/or EP300 gene.
13) The method of any one of paragraphs 1-12, further comprising a first step of detecting presence of at least one mutation in an epigenetic modifier selected from the group consisting of: EZH2, KMT2D, CREBPP, and/or EP300 gene before administering said compound.
14) The method of any one of paragraphs 1 to 13, further comprising administering an additional anti-cancer therapy to said subject.
15) A method for treating DLBCL, the method comprising:
diagnosing and/or selecting a subject as having DLBCL; and
administering to said subject a therapeutically effective amount of a compound targeting a KDM4/KDM5 family member.
16) The method of paragraph 15, wherein the compound is an inhibitor of a KDM4 family member.
17) The method of paragraph 15 or 16, wherein the compound is an inhibitor of KDM4A, KDM4B or KDM4C.
18) The method of any one of paragraphs 15-17, wherein the compound is a KDM4C inhibitor.
19) The method of any one of paragraphs 15-18, wherein the compound is 5-Chloro-2-[(E)-2-[phenyl(pyridin-2-yl)methylidene]hydrazin-1-yl]pyridine (JIB04).
20) The method of paragraph 15, wherein the compound is an inhibitor of KDM5A or KDM5B.
21) The method of paragraph 15, wherein the compound is an activator of KDM5C.
22) A method for treating cancer, the method comprising: administering a therapeutically effective amount of a compound targeting a KDM4 or KDM5 family member and a CDK7 inhibitor to a subject in need thereof, wherein the subject has at least one mutation in an epigenetic modifier selected from the group consisting of: EZH2, KMT2D, CREBPP, and EP300.
23) The method of paragraph 22, wherein the compound is an inhibitor of a KDM4 family member.
24) The method of paragraph 22 or 23, wherein the compound is an inhibitor of KDM4A, KDM4B or KDM4C.
25) The method of any one of paragraphs 22-24, wherein the compound is a KDM4C inhibitor.
26) The method of any one of paragraphs 22-25, wherein the compound is 5-Chloro-2-[(E)-2-[phenyl(pyridin-2-yl)methylidene]hydrazin-1-yl]pyridine (JIB04).
27) The method of paragraph 22, wherein the compound is an inhibitor of KDM5A or KDM5B.
28) The method of paragraph 22, wherein the compound is an activator of KDM5C.
29) The method of paragraph 22, wherein the CDK7 inhibitor is THZ-1.
30) The method of paragraphs 22-29, wherein the compound targeting a KDM4 or KDM5 family member and the CDK7 inhibitor are administered at a low dose.
31) A method for treating cancer, the method comprising: administering a therapeutically effective amount of a compound targeting a KDM4 or KDM5 family member and a BTK inhibitor to a subject in need thereof, wherein the subject has at least one mutation in an epigenetic modifier selected from the group consisting of: EZH2, KMT2D, CREBPP, and EP300.
32) The method of paragraph 31, wherein the compound is an inhibitor of a KDM4 family member.
33) The method of paragraph 31 and 32, wherein the compound is an inhibitor of KDM4A, KDM4B or KDM4C.
34) The method of any one of paragraphs 31-33, wherein the compound is a KDM4C inhibitor.
35) The method of any one of paragraphs 31-34, wherein the compound is 5-Chloro-2-[(E)-2-[phenyl(pyridin-2-yl)methylidene]hydrazin-1-yl]pyridine (JIB04).
36) The method of paragraph 31, wherein the compound is an inhibitor of KDM5A or KDM5B.
37) The method of paragraph 31, wherein the compound is an activator of KDM5C.
38) The method of paragraph 31, wherein the BTK inhibitor is Ibrutinib.
39) The method of any one of paragraphs 1-38, further comprising co-administering a second cancer therapy to the subject.
40) The method of claim any one of paragraphs 1-38, wherein the cancer is resistant to a chemotherapy or radiation therapy.

Additional, exemplary embodiments of the various aspects disclosed herein can be defined according to any of the following numbered paragraphs:
1. A method for treating cancer, the method comprising: administering a therapeutically effective amount of an inhibitor of a histone lysine demethylase (KDM) to a subject in need thereof, wherein the histone lysine demethylase is a KDM4 or KDM5 family member, and wherein:
   (i) the subject has at least one mutation in an epigenetic modifier selected from the group consisting of EZH2, KMT2D, CREBPP, and EP300;
   (ii) the subject has over-expression of at least one Ikaros family member;
   (iii) the subject has over-expression of KDM4A and/or KDM4C; and/or
   (iv) the subject has at least one mutation in canonical Wnt signaling.
2. The method of paragraph 1, wherein the subject has at least mutation in an epigenetic modifier selected from the group consisting of EZH2, KMT2D, CREBPP, and EP300.
3. The method of paragraph 1, wherein subject has over-expression of at least one Ikaros family member.
4. The method of paragraph 3, wherein the at least one Ikaros family member is IKZF1 and/or IKZF3.
5. The method of paragraph 1, wherein subject has over-expression of KDM4A and/or KDM4C.
6. The method of paragraph 1, wherein the subject has at least one mutation in canonical Wnt signaling.
7. The method of paragraph 1, wherein the subject has an activating Wnt-mutation.
8. The method of any one of paragraphs 1-7, further comprising selecting, prior to onset of treatment, a subject, wherein:
   (i) the subject has at least one mutation in an epigenetic modifier selected from the group consisting of EZH2, KMT2D, CREBPP, and EP300;
   (ii) the subject has over-expression of at least one Ikaros family member;
   (iii) the subject has over-expression of KDM4A and/or KDM4C; and/or
   (iv) the subject has at least one mutation in canonical Wnt signaling.
9. The method of any one of paragraphs 1-8, further comprising assaying, prior to onset of treatment, a biological sample from the subject for presence of the following:
   (i) at least one mutation in an epigenetic modifier selected from the group consisting of EZH2, KMT2D, CREBPP, and EP300;
   (ii) over-expression of at least one Ikaros family member;
   (iii) over-expression of KDM4A and/or KDM4C; and/or
   (iv) at least one mutation in canonical Wnt signaling.
10. The method of any one of paragraphs 1-9, wherein the inhibitor is an inhibitor of a KDM4 family member.
11. The method of paragraph 10, wherein the KDM4 family member is selected from the group consisting of KDM4A, KDM4B and KDM4C.
12. The method of paragraph 11, wherein the KDM4 family member is KDM4A and/or KDM4C.
13. The method of any one of paragraphs 1-12, wherein the inhibitor is an inhibitor of a KDM5 family member.
14. The method of paragraph 13, wherein the KDM5 family member is selected from the group consisting of KDM5A and KDM5B.
15. The method of any one of paragraphs 1-14, wherein the inhibitor is 5-Chloro-2-[(E)-2-[phenyl(pyridin-2-yl)methylidene]hydrazin-1-yl]pyridine (JIB04).
16. The method of any one of paragraphs 1-15, wherein the inhibitor is administered as a monotherapy.
17. The method of any one of paragraphs 1-15, further comprising co-administering a cyclin-dependent kinase (Cdk) inhibitor or a Bruton's tyrosine kinase (BTK) inhibitor to the subject.
18. The method of paragraph 17, wherein Cdk inhibitor or the BTK inhibitor is administered in an amount that is not effective to treat the cancer when the Cdk inhibitor or the BTK inhibitor is administered alone.
19. The method of paragraph 18, wherein the Cdk inhibitor is an inhibitor of Cdk7.
20. The method of any one of paragraphs 1-15, further comprising co-administering an inhibitor of B-cell receptor (BCR) signaling.
21. The method of any one of paragraphs 1-20, wherein the cancer results from increased activation of canonical WNT signaling.
22. The method of any one of paragraphs 1-21, wherein the cancer is selected from the group consisting of diffuse large B-cell lymphoma (DLBCL), colorectal cancer, acute myeloid leukemia (AML), thymoma, clear cell renal carcinoma, thyroid cancer, glioblastoma (glioblastoma multiforme, GBM), mesothelioma, ovarian cancer, and testicular cancer (Germ Cell Tumors).
23. The method of paragraph 22, wherein the cancer is DLBCL.
24. The method of any one of paragraphs 1-23, further comprising co-administering a second anti-cancer therapy to the subject.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

### Examples

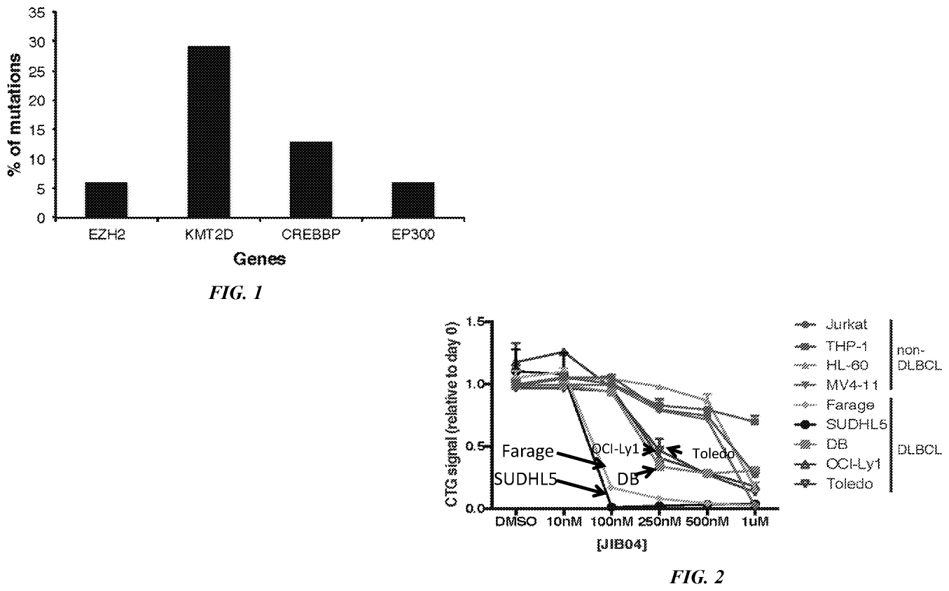

Stratification of patients based on gene expression profile divides DLBCL into two major subtypes—the germinal center B-cell (GCB) type and the activated B-cell (ABC) type (Alizadeh, A. A., et al., Nature 403, 503-511 (2000)). ABC type has a worse prognosis. Recent genome sequencing projects have uncovered an unexpectedly high frequency of mutations in the epigenetic regulators-most notably in KMT2D, EZH2 and CREBBP/EP300 as depicted in FIG. 1 (Lawrence, M. S., et al., Nature, 505, 495-501, (2014)). KMT2D catalyzes H3K4me2/3, and CREBBP/EP300 is responsible for histone acetylation; both histone post-translational modifications (PTMs) are associated with gene activation. EZH2, on the other hand, is responsible for H3K27me, a histone PTM associated with gene repression.

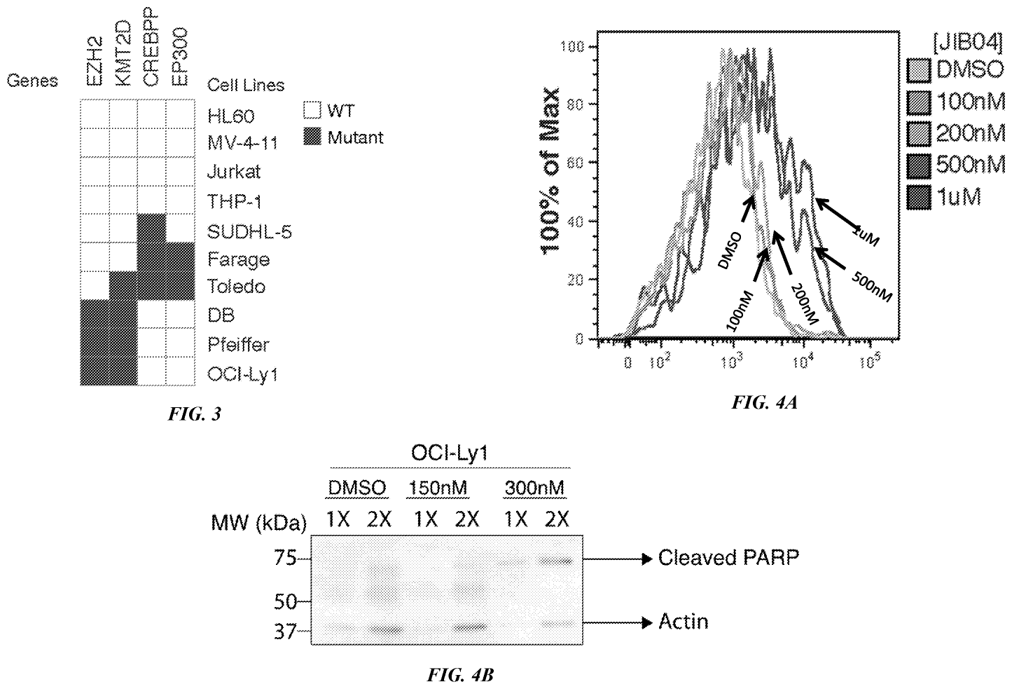

The inventors tested small molecule inhibitors targeting epigenetic modifiers against several lymphoma cell lines, and observed a striking sensitivity of DLBCL lines to JIB04, a pan-lysine demethylase (KDM) inhibitor. FIG. 2 is a dose response curve for 5 lymphoma and 4 non-lymphoma lines after treatment with JIB04 for 2 days. As shown in the FIG. 2, five different DLBCL lines (Farage, SUDHL5, DB, OCI-Ly1 and Toledo) are sensitive to JIB04 in sub-micromolar concentrations (EC50 values in sub-250 nM range) while four different leukemia lines (MV4-11, HL-60, THP-1 and Jurkat) showed an effective EC50 of about 1 μM. The current EZH2 inhibitor, which is in clinical trials for DLBCL, has an EC50 of >7-10 μM in OCI-Ly1 (Day 2), and the cells do not respond to the EZH2 inhibitor before four days of treatment (McCabe, et al., Nature, 492, 108-112 (2012)). Interestingly, all the sensitive lines had mutations in at least one of the four frequently mutated epigenetic modifiers, but none of the nonsensitive lines had a mutation in anyone of the these four frequently mutated epigenetic modifiers (FIG. 3).

To delineate the basis of reduced viability, annexin V staining was performed to measure apoptosis. An increase in annexin-V positive cells in a dose-dependent manner was observed as depicted in the plot shown in FIG. 4A. In addition, the inventors observed increased production of cleaved PARP (FIG. 4B).

JIB04 manifests in vitro selectivity towards KDM4 and KDM5 families. To test, if the DLBCL lines are sensitive to other Jumonji-inhibitors, and which KDM is being inhibited by JIB04, inventors tested five other Jumonji-inhibitors on DLBCL representative cell line OCI-Ly1: CPI-455 (Vinogradova, M., et al., Nature chemical biology, 12, 531-538 (2016)), KDM5-C70 (Johansson, C., et al., Nature chemical biology, 12, 539-545, (2016)), ML324 (Rai et. al Probe Reports from the NIH Molecular Libraries Program. Bethesda (Md.): National Center for Biotechnology Information (US); 2010-2012 Dec. 17 [updated 2013 Sep. 16]), GSK-J4 (Kruidenier et. al Nature, Nature volume 488, pages 404-408), and NSC636819 (Chu, C. H., et al. Journal of medicinal chemistry, 57, 5975-5985, (2014)). The data from these tests is plotted in FIG. 5 and shows that only JIB04 decreased OCI-Ly1 cell viability significantly in the range tested. While CPI-455, and KDM5C-70 inhibit various members of KDM5 family, NSC636819 inhibits KDM4A and KDM4B, GSK-J4 is purported to inhibit KDM5B, KDM5C, KDM6A and KDM6B, and ML324 is a KDM4E. This supports the notion that KDM4C is the most relevant cellular target for JIB04 in DLBCL cell lines.

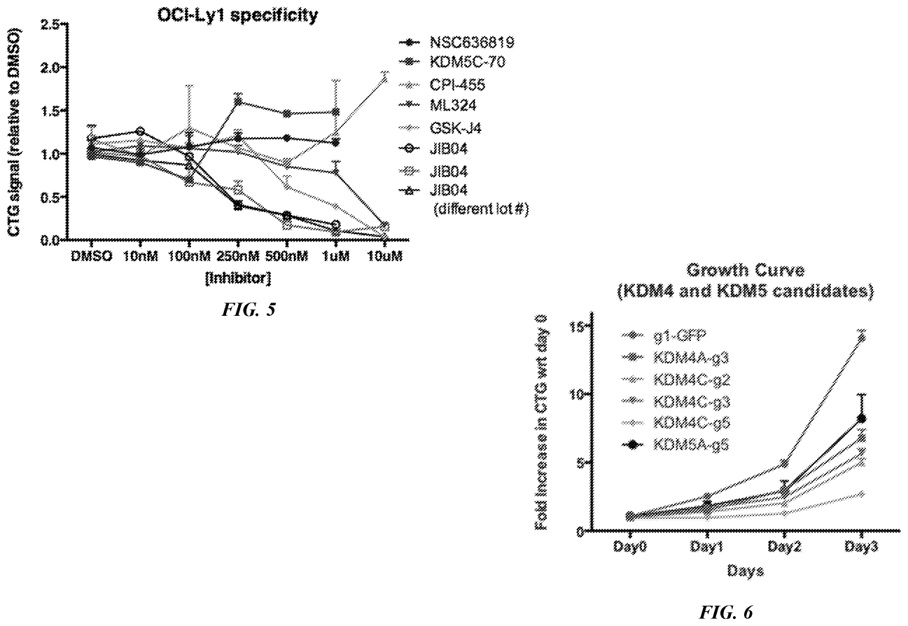

In addition, the inventors performed CRIPR/Cas knockdown. As shown in FIG. 6, multiple different gRNAs against KDM4C all resulted in significant growth-retardation for DLBCL cells in an in vitro proliferation assays addition.

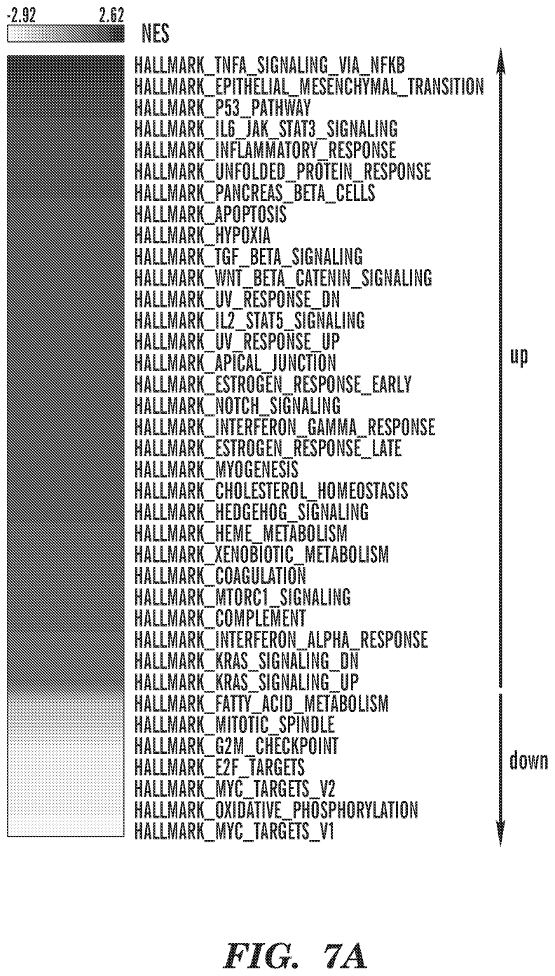

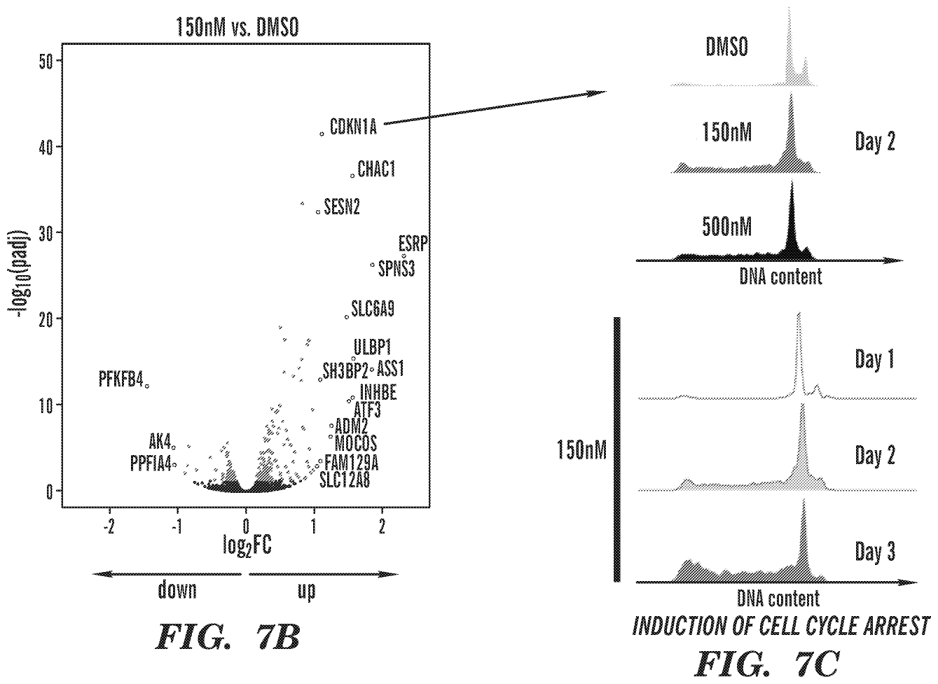

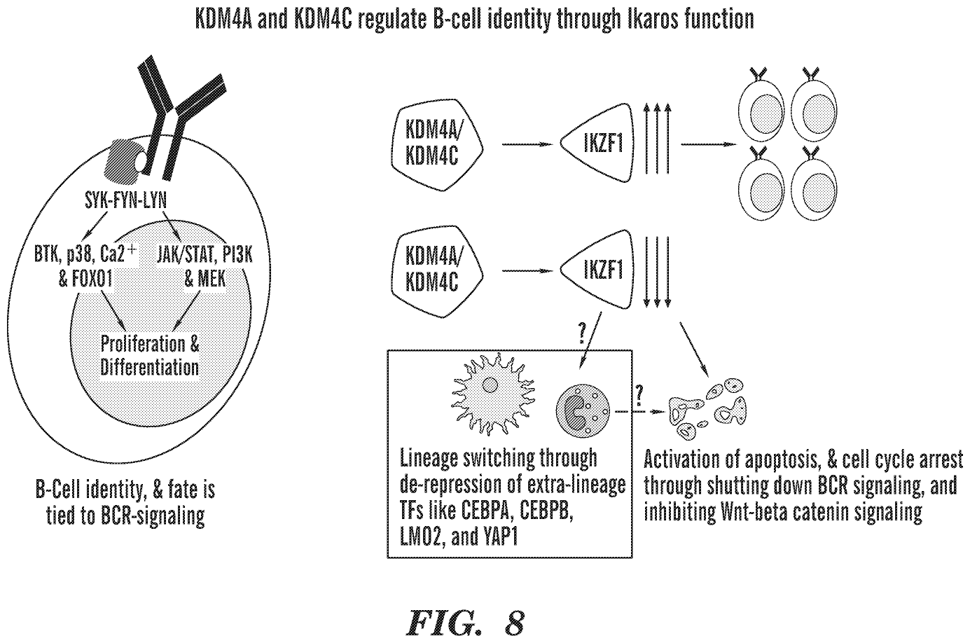

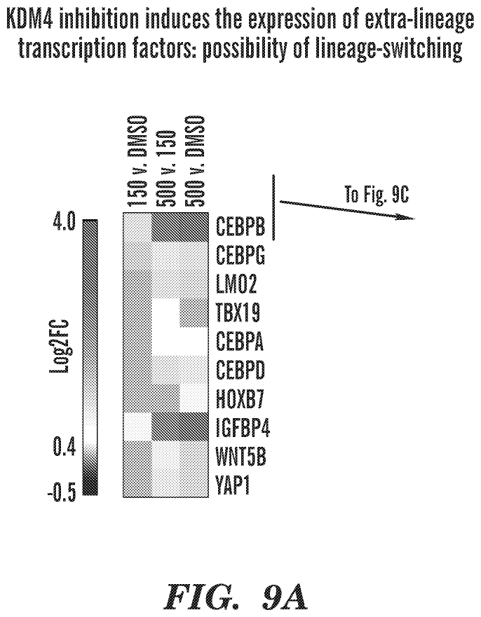

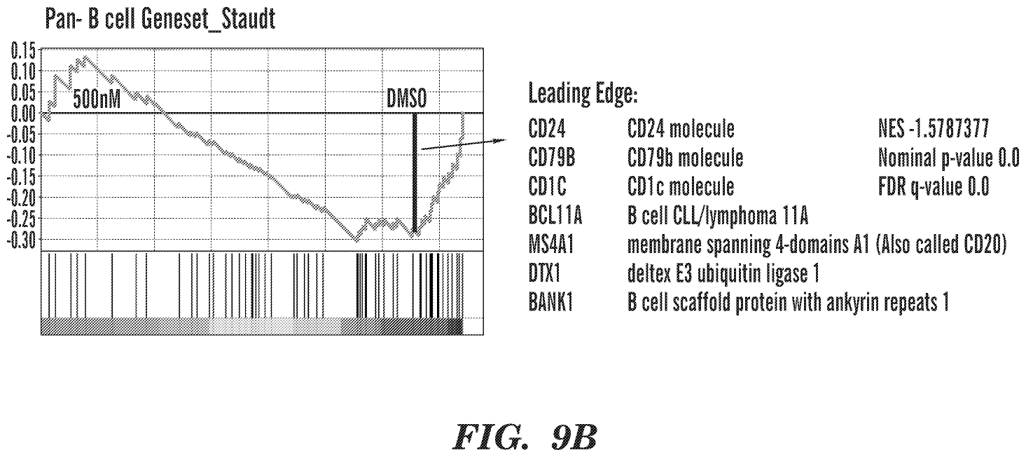

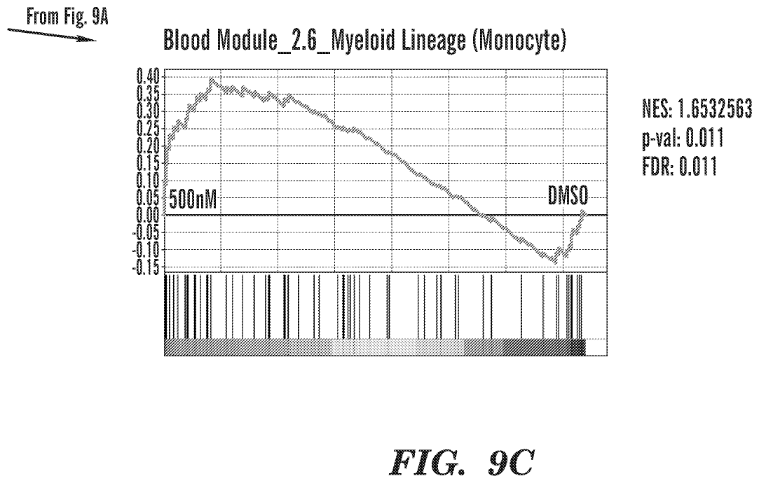

Transcriptomics experiments were performed on cells treated with JIB04 to understand the mechanistic underpinings of the discovered sensitivity of lymphoma cell lines to JIB04. Consistent with the annexin V staining experiments, the inventors observed an upregulation of “apoptosis” and “p53”-pathways while a downregulation of “E2F targets” and “Myc-targets”, demonstrating that treatment of lymphoma cell lines downregulates cell proliferation gene expression program while activating apoptotic programs (FIGS. 7A-7C). This is surprising in view of the fact that apoptotic programs are significantly down regulated, through multiple mechanisms during the tumorigenesis of DLBCL (Steinhardt, J. J. et al., Clinical cancer research: an official journal of the American Association for Cancer Research, 18, 4538-4548 (2012)). In addition, the inventors noticed that the cell cycle regulator, CDKN1A ((FIGS. 7B and 7C, was significantly upregulated. CDKN1A is a key regulator of G1-S arrest in human cells, upregulation of which results in cell cycle arrest (FIG. 7C).

The inventors also analyzed the gene expression profile of KDM4 and KDM5 family members with patient survival data (from a cohort of patients analyzed by Roche-MD Anderson), and observed that high expression of KDM4C in DLBCL patients correlated with poor prognosis (p=0.01) (FIG. 23).

### Methods for Examples

Cell Viability and Proliferation Assay:

For the Cell Titer Glo assays (CTG), between 3000-10,000 cells were plated in a 96 well plate (in triplicates). The cells were allowed to settle for 24 hours before either small molecule(s) or DMSO was added at indicated concentrations. The treatment was performed for indicated days (preferably 2 days for a “snap-shot” experiments). CTG signal was used to assess viability after 2 days according to manufacturer's protocol (Promega Corporation; Madison, Wis.).

When performing the comparative test of GSK126 and JIB04, daily readings were taken using the manufacturer's protocol for CTG signal after day 2 (for JIB04), and after day 5 (for GSK126).

For performing the proliferation assay in the presence of the small molecule, JIB04, 50,000 cells were plated in a 24 well plate in a total volume of 2 mL, and either DMSO or JIB04 (indicated concentrations) were added after 4 hours of cell plating. Samples were immediately collected for “day 0” time-point, and then at every 24 hours, samples were isolated for CTG assay.

ShRNA Knockdown:

shRNAs for each candidate gene was obtained from SIGMA TRC2 collection (Sigma-Aldrich; St. Louis, Mo.), and lentiviruses were made using a standard protocol known to one skilled in the art. Lentivirus infection was carried out using Lipofectamine 9 reagent from Roche (Basel, Switzerland), according to manufacturer's protocol. After 24 hours on lentivirus transduction, cells were grown in media containing puromycin to select for transduced clones. Knock-down efficiency was assessed by qPCR analysis of relevant genes.

Annexin V Staining:

APC-Annexin V was obtained from BD Pharmingen (Franklin Lakes, N.J.), and Annexin V staining was performed as described by the manufacturer.

Tanscriptomics Analysis:

Three independent cultures of OCI-Ly1 cell lines were grown till the cultures achieved the cell numbers of approximately 1-1.5*10\hat{ }6 cells/ml, and then cells were treated with either DMSO or two different JIB04 concentrations (150 nM and 500 nM). RNA was isolated using Trizol method, followed by library preparation using NEBUltraII kit (New England Biolabs; Ipswich, Mass.). After high throughput sequencing of the libraries, FastQc was used for filtering and quality control of the reads followed by read alignment to hg19 assembly of the human genome. Reads were counted by TopHat, differential gene expression analysis was performed in Rstudio. GSEA was performed using the desktop client from the Broad Institute, and the GSEA output was visualized using Rstudio. Results are shown in FIGS. 7A, 9B, 9C and 21C.

Cell Cycle Analysis:

Propidium Iodide Flow Cytometry Kit from Abcam was used to perform cell cycle analysis using propidium iodide staining according to manufacturer's protocol. Results are shown in FIG. 7C.

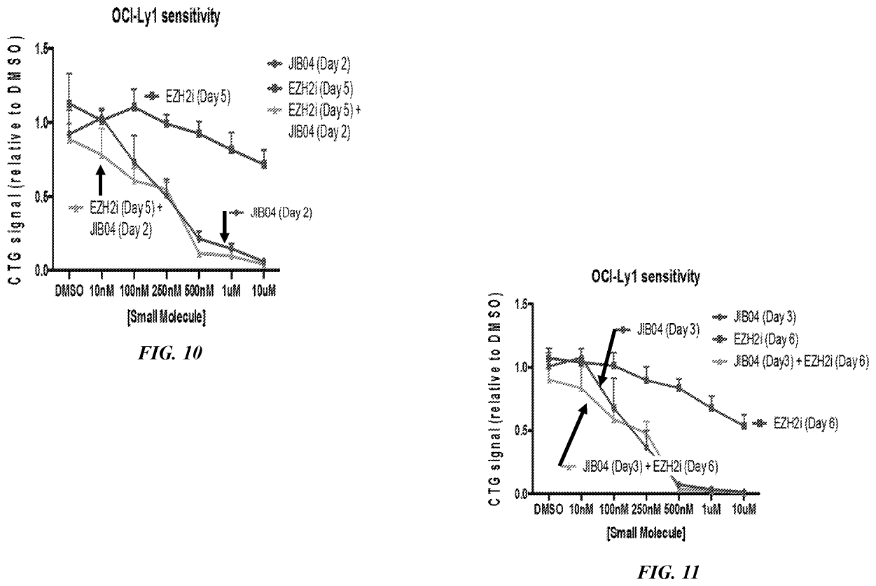

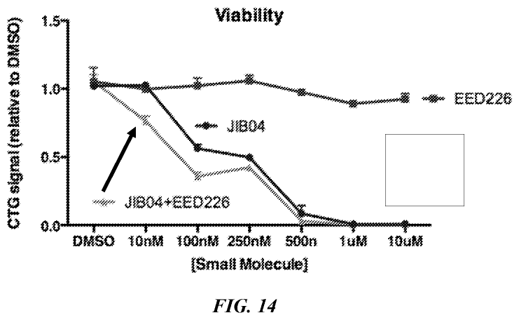

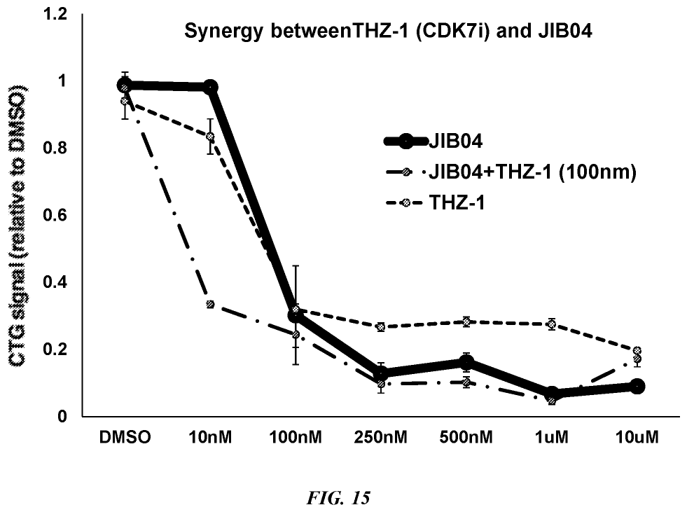

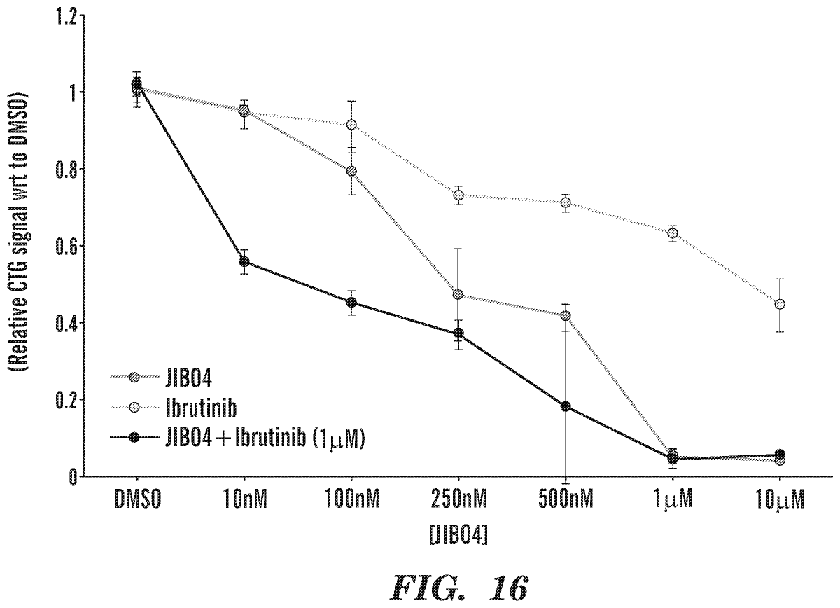

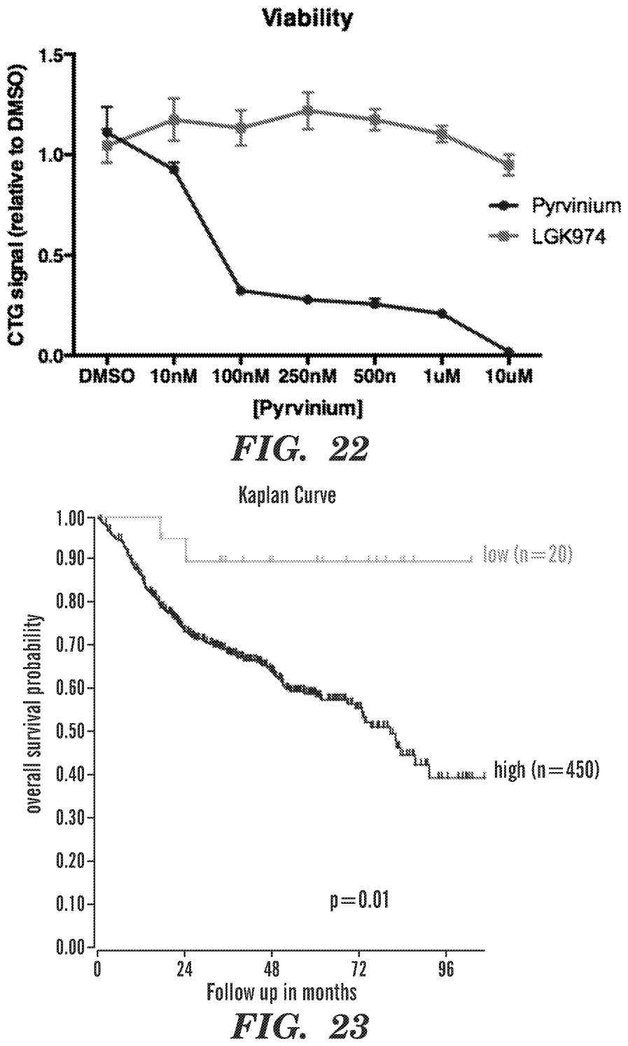

For OCI-Ly1 sensitivity assay, OCI-Ly1 cells were grown at a density of 1-3×10\hat{ }6 cells/mL and 3000-10,000 cells were plated in a 96-well plate (in triplicates). The cells were allowed to settle for 24 hours before either the indicated small molecule(s) or DMSO was added at indicated concentrations. CTG signal was used to assess viability after 2 days according to manufacturer's protocol (Promega Corporation; Madison, Wis.). Small molecules used in each experiment are stated in the respective figures along with their concentrations. Results are shown in FIGS. 10, 11, 13, 14, 15, 16 and 22.

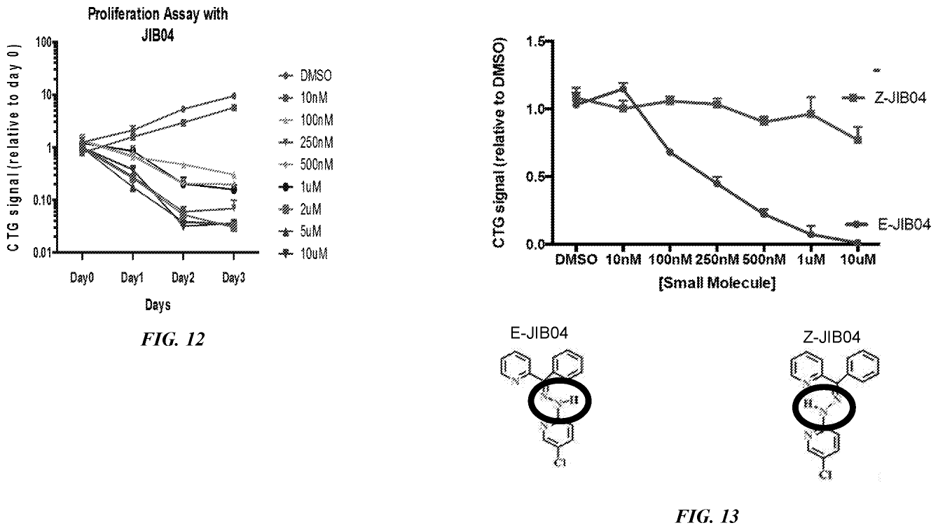

For performing the proliferation assay in the presence of the small molecule, 50,000 cells were plated in a 24 well plate in a total volume of 2 mL, and either DMSO or JIB04 (indicated concentrations) were added after 4 hours of cell plating. Samples were immediately collected for “day 0” time-point, and then at every 24 hours, samples were isolated for CTG assay. Plate readings were normalized to day 0 reading and plotted on Graphpad PRISM. Results are shown in FIG. 12.

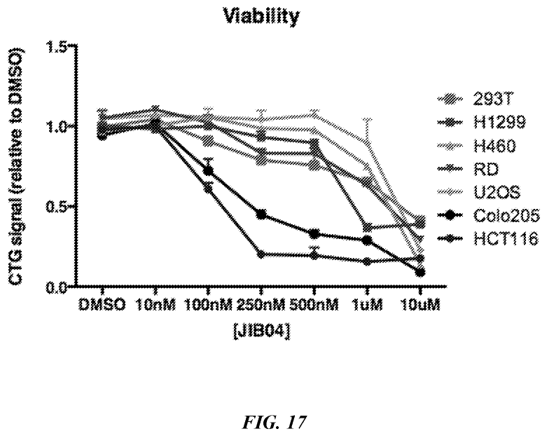

Effect of JIB04 on various cell lines was assayed as follows. Indicated human cell lines were grown in 10 cm dishes to a density of 1-3×10\hat{ }6 cells/mL, and maintained in accordance with the guidelines of American Type Culture Collection (ATCC). 3000-500 cells were then plated in a 96-well plate and they were allowed to settle for 24 hours before either small molecule (JIB04) or DMSO were added at indicated concentrations. CTG signal was used to assess viability after 2 days according to manufacturer's protocol (Promega Corporation; Madison, Wis.). Results are shown in FIG. 17. It is noted that H1299, a lung cancer cell line, harbors silent KMT2D mutation, and HCT116, a colon cancer cell line, harbors KMT2D and EP300 mutations.

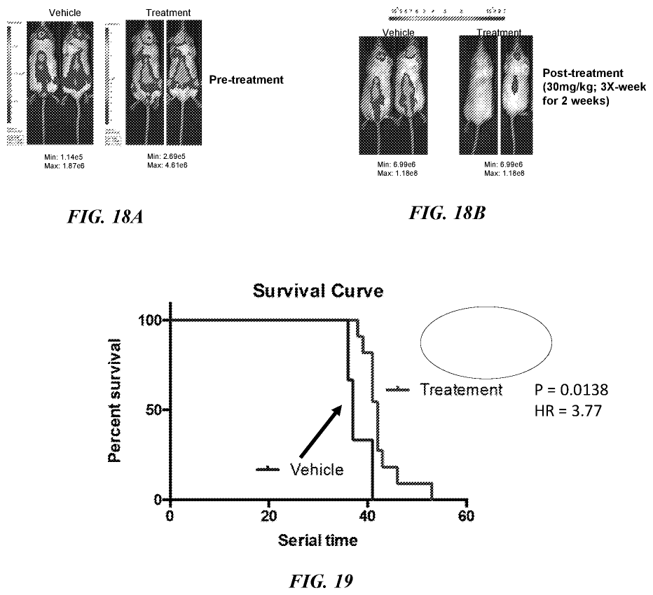

For in vivo studies, Ly1 cells, with a luciferase reporter or with a reporter, were administered to Nod/SCID− mice (NSG) (6-8 weeks) were used for tail vein injection. Mice administered the Ly1 cells comprising the luciferase reporter were imaged every week after injection using the IVIS Spectrum In Vivo Imaging System (Perkin Elmer). At 3 weeks post-injection, 8 mice with comparable bioluminescence (in the range of a e05 to e06) were chosen for treatment, 4 in each group (vehicle vs. treatment). JIB04 was administered three times a week, for two weeks, via intra-peritoneal injection in a mix of 90% sesame oil and 10% DMSO mix (vehicle). Mice were imaged each week and images from pre-treatment and 2-weeks after start of treatment with 30 mg/kg (3× a week) JIB04 are shown in FIG. 18A (pre-treatment) and FIG. 18B (post-treatment).

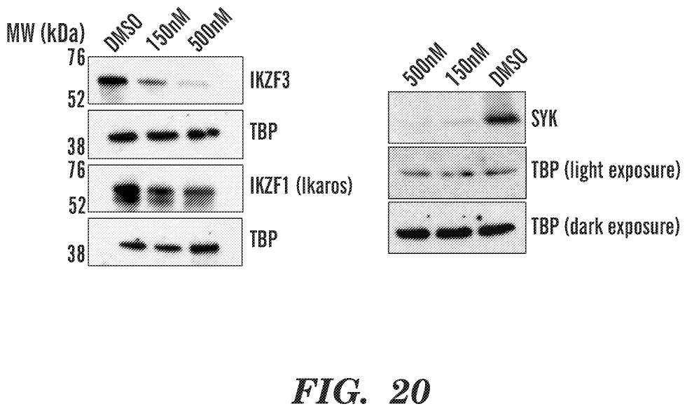

Mice administered Ly1 cells without the luciferase reporter were monitored post administration, and fraction surviving was plotted (FIG. 19) using the Kaplan Meier Survival Analysis of Graphpad Prism.

Effect of JIB-04 on Ikaros family members and proximal components of BCR signaling pathway was assayed as follows: One million cells growing under indicated conditions (DMSO or JIB04 treatment; 100 nM or 500 nM) were pelleted by centrifugation at 1000 rpm for 5 min, and then resuspended in RIPA buffer with protease and phosphatase inhibitors. The cell suspension was left on ice for 20 min, and then sonicated using Diagenode water-bath sonicator (15 s. on; 30 s. off, for 15 cycles at max power). After sonication, cell extract was spun at 12000 rpm for 20 min, and the supernatant was collected for immunoblotting. Immunoblotting was performed using standard methods, and the blots were probed using antibodies recognizing indicated proteins. Results are shown in FIG. 20.

Chip-Seq Analysis:

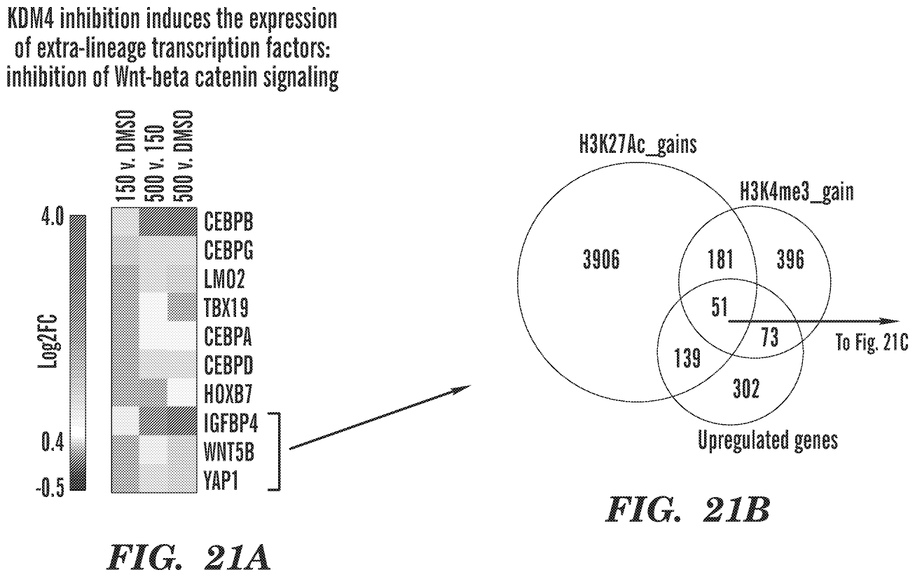

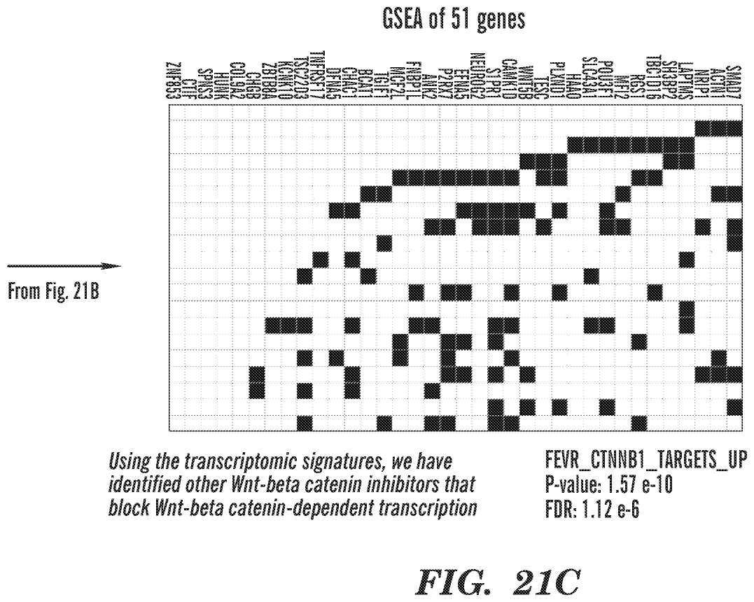

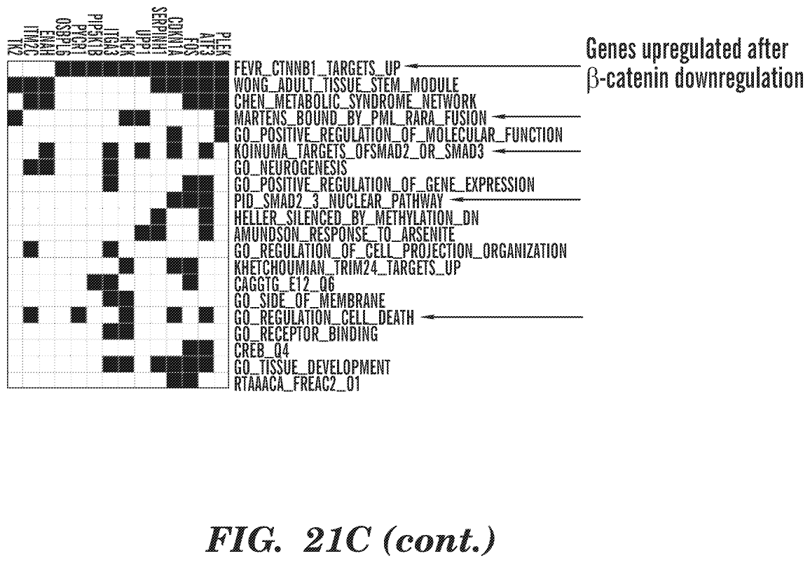

Cells were fixed with 1% formaldehyde for 10 minutes at room temperature before termination with 0.125 M glycine. Cells were then lysed in ChIP buffer (0.6% SDS, 10 mM EDTA, and 50 mM Tris-HCl, pH 8.1) and cross-linked chromatin was sonicated to obtain DNA fragments of 300-800 bp. Chromatin was then centrifuged (10 min, 4° C., 13,000 rpm) and the supernatant was diluted 6-fold in ChIP dilution buffer (0.01% SDS, 1.1% Triton X-100, 1.2 mM EDTA, 16.7 mM Tris-HCl pH 8, and 167 mM NaCl) with protease inhibitors (Roche Diagnostics). Diluted chromatin was incubated with 5 μg of primary antibody overnight at 4C under agitation. Antibodies used were as follows: H3K27Ac (Abcam), H3K27me3 (Abcam), H3K4me1 (Abcam). Antibodies were pelleted using 50 μL of dynabeads (ThermoFisher Scientific). Beads were sequentially washed once with the ChIP wash buffer 1 (0.1% SDS, 1% Triton X-100, 2 mM EDTA, 20 mM Tris-HCl pH 8, and 150 mM NaCl), the ChIP wash buffer 2 (0.1% SDS, 1% Triton X-100, 2 mM EDTA, 20 mM Tris-HCl pH 8, and 500 mM NaCl), the ChIP wash buffer 3 (0.25 M LiCl, 1% IGEPAL-CA630, 1% deoxycholic acid, 1 mM EDTA, 10 mM Tris pH 8), and finally the ChIP wash buffer 4 (10 mM Tris-HCl, 1 mM EDTA). Chromatin was eluted using ChIP Elution Buffer (1% SDS, 100 mM NaHCO₃). The cross-link between protein and DNA was reversed by heat incubation (65° C., overnight), and proteins were then digested with proteinase K. DNA was recovered by phenol-chloroform extraction and ethanol precipitation in presence of 20 μg of glycogen (Sigma Aldrich). ChIP-seq libraries were prepared using NEBNext DNA library preparation reagents (E6040); the protocol and reagents concentrations described in the Illumina Multiplex ChIP-seq DNA sample Prep Kit. Libraries were indexed using a single indexed PCR primer. Libraries were quantified by Qubit (Invitrogen), and sequenced using a HiSeq 2000 (Illumina) to generate 50 bp single end reads. The significantly enriched peaks were determined by Model-Based Analysis of ChIP-Seq (MACS) package based on a p-value≤10⁻⁵ and a false discovery rate (FDR) cut-off≤0.05. Results are shown in FIG. 21B.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

SEQUENCES

SEQ ID NO: 1
KDM4A
MASESETLNPSARIMTYPTMEEFRNFSRYIAYIESQGAHRAGLAKVVPPKEWKPRASY
DDIDDLVIPAPIQQLVTGQSGLFTQYNIQKKAMTVREFRKIANSDKYCTPRYSEFEELER
KYWKNLTFNPPIYGADVNGTLYEKHVDEWNIGRLRTILDLVEKESGITIEGVNTPYLYFG
MWKTSFAWHTEDMDLYSINYLHFGEPKSWYSVPPEHGKRLERLAKGFFPGSAQSCEAF
LRHKMTLISPLMLKKYGIPFDKVTQEAGEFMITFPYGYHAGFNHGFNCAESTNFATRRW
IEYGKQAVLCSCRKDMVKISMDVFVRKFQPERYKLWKAGKDNTNDHTLPTPEAAEFL
KESELPPRAGNEEECPEEDMEGVEDGEEGDLKTSLAKHRIGTKRHRVCLEIPQEVSQSEL
FPKEDLSSEQYEMTECPAALAPVRPTHSSVRQVEDGLTFPDYSDSTEVKFEELKNVKLEE
EDEEEEQAAAALDLSVNPASVGGRLVFSGSKKKSSSSLGSGSSRDSISSDSETSEPLSCRA
QGQTGVLTVHSYAKGDGRVTVGEPCTRKKGSAARSFSERELAEVADEYMFSLEENKKS
KGRRQPLSKLPRHHPLVLQECVSDDETSEQLTPEEEAEETEAWAKPLSQLWQNRPPNFE
AEKEFNETMAQQAPHCAVCMIFQTYHQVEFGGFNQNCGNASDLAPQKQRTKPLIPEMC
FTSTGCSTDINLSTPYLEEDGTSILVSCKKCSVRVHASCYGVPPAKASEDWMCSRCSANA
LEEDCCLCSLRGGALQRANDDRWVHVSCAVAILEARFVNIAERSPVDVSKIPLPRFKLK
CIFCKKRRKRTAGCCVQCSHGRCPTAFHNTSCAQAAGVMMQPDDWPFVVFITCFRHKIP
NLERAKGALQSITAGQKVISKHKNGRFYQCEVVRLTTETFYEVNFDDGSFSDNLYPEDI
VSQDCLQFGPPAEGEVVQVRWTDGQVYGAKFVASHPIQMYQVEFEDGSQLVVKRDDV
YTLDEELPKRVKSRLSVASDMRFNEIFTEKEVKQEKKRQRVINSRYREDYIEPALYRAIM
E
SEQ ID NO: 2
KDM4B
MGSEDHGAQNPSCKIMTFRPTMEEFKDFNKYVAYIESQGAHRAGLAKIIPPKEWKPRQT
YDDIDDVVIPAPIQQVVTGQSGLFTQYNIQKKAMTVGEYRRLANSKEYCTPRHQDFDDL
ERKYWKNLTFVSPIYGADISGSLYDDDVAQWNIGSLRTILDMVERECGTIIEGVNTPYLY
FGMWKTTFAWHTEDMDLYSINYLHFGEPKSWYAIPPEHGKRLERLAIGFFPGSSQGCDA
FLRHKMTLISPIILKKYGIPFSRITQEAGEFMITFPYGYHAGFNHGFNCAESTNFATLRWID
YGKVATQCTCRKDMVKISMDVFVRILQPERYELWKQGKDLTVLDHTRPTALTSPELSS
WSASRASLKAKLLRRSHRKRSQPKKPKPEDPKFPGEGTAGAALLEEAGGSVKEEAGPEV
DPEEEEEEPQPLPHGREAEGAEEDGRGKLRPTKAKSERKKKSFGLLPPQLPPPPAHFPSEE
ALWLPSPLEPPVLGPGPAAMEESPLPAPLNVVPPEVPSEELEAKPRPIIPMLYVVPRPGKA
AFNQEHVSCQQAFEHFAQKGPTWKEPVSPMELTGPEDGAASSGAGRMETKARAGEGQ
APSTFSKLKMEIKKSRRHPLGRPPTRSPLSVVKQEASSDEEASPFSGEEDVSDPDALRPLL
SLQWKNRAASFQAERKFNAAAARTEPYCAICTLFYPYCQALQTEKEAPIASLGEGCPAT
LPSKSRQKTRPLIPEMCFTSGGENTEPLPANSYIGDDGTSPLIACGKCCLQVHASCYGIRP
ELVNEGWTCSRCAAHAWTAECCLCNLRGGALQMTTDRRWIHVICAIAVPEARFLNVIE
RHPVDISAIPEQRWLKCVYCRKRMKKVSGACIQCSYEHCSTSFHVTCAHAAGVLMEP
DDWPYVVSITCLKHKSGGHAVQLLRAVSLGQVVITKNRNGLYYRCRVIGAASQTCYEV
NFDDGSYSDNLYPESITSRDCVQLGPPSEGELVELRWTDGNLYKAKFISSVTSHIYQVEF
EDGSQLTVKRGDIFTLEEELPKRVRSRLSLSTGAPQEPAFSGEEAKAAKRPRVGTPLATE
DSGRSQDYVAFVESLLQVQGRPGAPF
SEQ ID NO: 3
KDM4C
MEVAEVESPLNPSCKIMTFRPSMEEFREFNKYLAYMESKGAHRAGLAKVIPPKEWKPRQ
CYDDIDNLLIPAPIQQMVTGQSGLFTQYNIQKKAMTVKEFRQLANSGKYCTPRYLDYED
LERKYWKNLTFVAPIYGADINGSIYDEGVDEWNIARLNTVLDVVEEECGISIEGVNTPYL
YFGMWKTTFAWHTEDMDLYSINYLHFGEPKSWYAIPPEHGKRLERLAQGFFPSSSQGC
DAFLRHKMTLISPSVLKKYGIPFDKITQEAGEFMITFPYGYHAGFNHGFNCAESTNFATV
RWIDYGKVAKLCTCRKDMVKISMDIFVRKFQPDRYQLWKQGKDIYTIDHTKPTPASTPE
VKAWLQRRRKVRKASRSFQCARSTSKRPKADEEEEVSDEVGAEVPNPDSTDDLKVS
EKSEAAVKLRNTEASSEEESSASRMQVEQNLSDHIKLSGNSCLSTSVTEDIKTEDDKAYA
YRSVPSISSEADDSIPLSSGYEKPEKSDPSELSWPKSPESCSSVAESNGVLTEGEESDVESH
GNGLEPGEIPAVPSGERNSFKVPSIAEGENKTSKSWRHPLSRPPARSPMTLVKQQAPSDE
ELPEVLSIEEEVEETESWAKPLIHLWQTKSPNFAAEQEYNATVARMKPHCAICTLLMPY
HKPDSSNEENDARWETKLDEVVTSEGKTKPLIPEMCFIYSEENIEYSPPNAFLEEDGTSLL
ISCAKCCVRVHASCYGIPSHEICDGWLCARCKRNAWTAECCLCNLRGGALKQTKNNK
WAHVMCAVAVPAEVRFTNVPERTQIDVGRIPLQRLKLGRLGI
SEQ ID NO: 4
KDM4D
METMKSKANCAQNPNCNIMIFHPTKEEFNDFDKYIAYMESQGAHRAGLAKIIPPKEWK
ARETYDNISEILIATPLQQVASGRAGVFTQYHKKKKAMTVGEYRHLANSKKYQTPPHQ
NFEDLERKYWKNRIYNSPIYGADISGSLFDENTKQWNLGHLGTIQDLLEKECGVVIEGV
NTPYLYFGMWKTTFAWHTEDMDLYSINYLHLGEPKTWYVVPPEHGQRLERLARELFPG
SSRGCGAFLRHKVALISPTVLKENGIPFNRITQEAGEFMVTFPYGYHAGFNHGFNCAEAI
NFATPRWIDYGKMASQCSCGEARVTFSMDAFVRILQPERYDLWKRGQDRAVVDHMEP
RVPASQELSTQKEVQLPRRAALGLRQLPSHWARHSPWPMAARSGTRCHTLVCSSLPRRS
AVSGTATQPRAAAVHSSKKPSSTPSSTPGPSAQIIHPSNGRPPQKLRAQELTLQTP
AKRPLLAGTTCTASGPEPEPLPEDGALMDKPVPLSPGLQHPVKASGCSWAPVP
SEQ ID NO: 5
KDM5A
MAGVGPGGYAAEFVPPPECPVFEPSWEEFTDPLSFIGRIRPLAEKTGICKIRPPKDWQPPF
ACEVKSFRFTPRVQLNELEAMTRVRLDFLDQLAKFWELQGSTLKIPVVERKILDLYAL
SKIVASKGGFEMVTKKWSKVGSRLGYLPGKGTGSLLKSHYERILYPYELFQSGVSLM
GVQMPNLDLKEKVEPEVLSTDTQTSPEPGTRMNILPKRTRRVKTQSESGDVSRTELKK
LQIFGAGPKVVGLAMGTKDKEDEVTRRRKVTNRSDAFNMQMRQRKGTLSVNFVDLYV
CMFCGRGNNEDKLLLCDGCDDSYHTFCLIPPLPDVPKGDWRCPKCVAEECSKPREAFGF
EQAVREYTLQSFGEMADNFKSDYFNMPVHMVPTELVEKEFWRLVSSIEEDVIVEYGADI
SSKDFGSGFPVKDGRRKILPEEEEYALSGWNLNNMPVLEQSVLAHINVDISGMKVPWLY
VGMCFSSFCWHIEDHWSYSINYLHWGEPKTWYGVPSHAAEQLEEVMRELAPELFESQP
DLLHQLVTIMNPNVLMEHGVPVYRTNQCAGEFVVTFPRAYHSGFNQGYNFAEAVNFCT
ADWLPIGRQCVNHYRRLRRHCVFSHEELIFKMAADPECLDVGLAAMVCKELTLMTEEE
TRLRESVVQMGVLMSEEEVFELVPDDERQCSACRTTCFLSALTCSCNPERLVCLYHPTD
LCPCPMQKKCLRYRYPLEDLPSLLYGVKVRAQSYDTWVSRTEALSANFNHKKDLIEL
RVMLEDAEDRKYPENDLFRKLRDAVKEAETCASVAQLLLSKKQKHRQSPDSGRTRTKL
TVEELKAFVQQLFSLPCVISQARQVKNLLDDVEEFHERAQEAMMDETPDSSKLQMLID
MGSSLYVELPELPRLKQELQQARWLDEVRLTLSDPQQVTLDVMKKLIDSGVGLAPHHA
VEKAMAELQELLTVSERWEEKAKVCLQARPRHSVASLESIVNEAKNIPAFLPNVLSLKE
ALQKAREWTAKVEAIQSGSNYAYLEQLESLSAKGRPIPVLEALPQVESQVAAARAWR
ERTGRTFLKKNSSHTLLQVLSPRTDIGVYGSGKNRRKKVKELIEKEKEKDLDLEPLSDLE
EGLEETRDTAMVVAVFKEREQKEIEAMHSLRAANLAKMTMVDRIEEVKFCICRKTASG
FMLQCELCKDWFHNSCVPLPKSSSQKKGSSWQAKEVKFLCPLCMRSRRPRLETILSLLV
SLQKLPVRLPEGEALQCLTERAMSWQDRARQALATDELSSALAKLSVLSQRMVEQAAR
EKTEKIISAELQKAAANPDLQGHLPSFQQSAFNRVVSSVSSSPRQTMDYDDEETDSDEDI
RETYGYDMKDTASVKSSSSLEPNLFCDEEIPIKSEEVVTHMWTAPSFCAEHAYSSASKSC
SQGSSTPRKQPRKSPLVPRSLEPPVLELSPGAKAQLEELMMVGDLLEVSLDETQHIWRIL
QATHPPSEDRFLHIMEDDSMEEKPLKVKGKDSSEKKRKRKLEKVEQLFGEGKQKSKEL
KKMDKPRKKKLKLGADKSKELNKLAKKLAKEEERKKKKEKAAAAKVELVKESTEKKR
EKKVLDIPSKYDWSGAEESDDENAVCAAQNCQRPCKDKVDWVQCDGGCDEWFHQVC
VGVSPEMAENEDYICINCAKKQGPVSPGPAPPPSFIMSYKLPMEDLKETS
SEQ ID NO: 6
KDM5B
MEAATTLHPGPRPALPLGGPGPLGEFLPPPECPVFEPSWEEFADPFAFIHKIRPIAEQTGIC
KVRPPPDWQPPFACDVDKLHFTPRIQRLNELEAQTRVKLNFLDQIAKYWELQGSTLKIP
HVERKILDLFQLNKLVAEEGGFAVVCKDRKWTKIATKMGFAPGKAVGSHIRGHYERIL
NPYNLFLSGDSLRCLQKPNLTTDTKDKEYKPHDIPQRQSVQPSETCPPARRAKRMRAER
QSLAVLPRLECSGAILAHCNLRLLDSSNSSASASQAMNIKIEPEETTEARTHNLRRRMGC
PTPKCENEKEMKSSIKQEPIERKDYIVENEKEKPKSRSKKATNAVDLYVCLLCGSGNDE
DRLLLCDGCDDSYHTFCLIPPLHDVPKGDWRCPKCLAQECSKPQEAFGFEQAARDYTLR
TFGEMADAFKSDYFNMPVHMVPTELVEKEFWRLVSTIEEDVTVEYGADIASKEFGSGFP
VRDGKIKLSPEEEEYLDSGWNLNNMPVMEQSVLAHITADICGMKLPWLYVGMCFSSFC
WHIEDHWSYSINYLHWGEPKTWYGVPGYAAEQLENVMKKLAPELFVSQPDLLHQLVTI
MNPNTLMTHEVPVYRTNQCAGEFVITFPRAYHSGFNQGFNFAEAVNFCTVDWLPLGRQ
CVEHYRLLHRYCVFSHDEMICKMASKADVLDVVVASTVQKDMAIMIEDEKALRETVR
KLGVIDSERMDFELLPDDERQCVKCKTTCFMSAISCSCKPGLLVCLHHVKELCSCPPYK
YKLRYRYTLLDLYPMMNALKLRAESYNEWALNVNEALEAKINKKKSLVSFKALIEESE
MKKFPDNDLLRHLRLVTQDAEKCASVAQQLLNGKRQTRYRSGGGKSQNQLTVNELRQ
FVTQLYALPCVLSQTPLLKDLLNRVEDFQQHSWKLLSEETPSAAELQDLLDVSFEFDVEL
PQLAEMRIRLEQARWLEEVQQACLDPSSLTLLDDMRRLIDLGVGLAPYSAVEKAMARLQ
ELLTVSEHWDDKAKSLLKARPRHSLNSLATAVKEIEEIPAYLPNGAALKDSVQRARDWL
QDVEGLQAGGRVPVLDTLIELVTRGRSIPVHLNSLPRLETLVAEVQAWKECAVNTFLTE
NSPYSLLEVLCPRCDIGLLGLKRKQRKLKEPLPNGKKKSTKLESLSDLERALTESKETAS
AMATLGEARLREMEALQSLRLANEGKLLSPLQDVDIKICLCQKAPAAPMIQCELCRDAF
HTSCVAVPSISQGLRIWLCPHCRRSEKPPLEKILPPLLASLQRIRVRLPEGDALRYMIERTV
NWQHRAQQLLSSGNLKFVQDRVGSGLLYSRWQASAGQVSDTNKVSQPPGTTSFSLPDD
WDNRTSYLHSPFSTGRSCIPLHGVSPEVNELLMEAQLLQVSLPEIQELYQTLLAKPSPAQ
QTDRSSPVRPSSEKNDCCRGKRDGINSLERKLKRRLEREGLSSERWERVKKMRTPKKKK
IKLSHPKDMNNFKLERERSYELVSAETHSLPSDTSYSEQEDSEDEDAICPAVSCLQPEG
DEVDWVQCDGSCNQWFHQVCVGVSPEMAEKEDYICVRCTVKDAPSRK
SEQ ID NO: 7
KDM5C
MEPGSDDFLPPPECPVFEPSWAEFRDPLGYIAKIRPIAEKSGICKIRPPAIVVEEGGYEAIC
KDRRWARVAQRLNYPPGKNIGSLLRSHYERIVYPYEMYQSGANLVQCNTRPFDNEEKD
KEYKPHSIPLRQSVQPSKFNSYGRRAKRLQPDPEPTEEDIEKNPELKKLQIYGAGPKMMG
LGLMAKDKTLRKKDKEGPECPPTVVVKEELGGDVKVESTSPKTFLESKEELSHSPEPCT
KMTMRLRRNSHSNAQFIESYVCRMCSRGDEDDKLLLCDGCDDNYHIFCLLPPLPEIPKGV
WRCPKCVMAECKRPPEAFGFEQATREYTLQSFGEMADSFKADYFMPVHMVPTELVE
KEFWRLVNSIEEDVTVEYGADIHSKEFGSGFPVSDSKRHLTPEEEEYATSGWNLNVMPV
LEQSVLCHINADISGMKVPWLYVGMVFSAFCWHIEDHWSYSINYLHWGEPKTWYGVPS
LAAEHLEEVMKKLTPELFDSQPDLLHQLVTLMNPNTLMSHGVPVVRTNQCAGEFVITFP
RAYHSGFNQYNFAEAVNFCTADWLPAGRQCIEHYRRLRRYCVFSHEELICKMAACPE
KLDLNLAAAVHKEMFIMVQEERRLRKALLEKGITEAEREAFELLPDDERQCIKCKTTCF
LSALACYDCPDGLVCLSHINDLCKCSSSRQYLRYRYTLDELPAMLHKLKVRAESFDTW
ANKVRVALEVEDGRKRSLEELRALESEARERRFPNSELLQQLKNCLSEAEACVSRALGL
VSGQEAGPHRVAGLQMTLTELRAFLDMNNLPCAMHQIGDVKGVLEQVEAYQAEARE
ALASLPSSPGLLQSLLERGRQLGVEVPEAQQLQRQVEQARWLDEVKRTLAPSAEEGTLA
VMRGLLVAGASVAPSPAVDKAQAELQELLTIAERWEEKAHLCLEARQKHPPATLEAIIR
EAENIPVHLNIQALKEALAKARAWIADVDEIQNGDHYPCLDDLEGLVAVGRDLPVGLE
ELRQLELQVLTAHSWREKASKTFLKKNSCYTLLEVLCPCADAGSDSTKRSRWMEKELG
LYKSDTELLGLSAQDLRDPGSVIVAFKEGEWKEKEGILQLRRTNSAKPSPLASSSTASSTT
SICVCGQVLAGAGALQCDLCQDWFHGRCVSVPRLLSSPRPNPTSSPLLAWWEWDTKFL
CPLCMRSRRPRLETILALLVALQRLPVRLPEGEALQCLTERAISWQGRARQALASEDVT
ALLGRLAELRQRLQAEPRPEEPPNYPAAPASDPLREGSGKDMPKVQGLLENGDSVTSPE
KVAPEEGSDLELLSSLLPQLTGPVLELPEATRAPLEELMMEGDLLEVTLDENHSIPESLDF
CILTPRYCSDLSSWGPAPGVCPPW
SEQ ID NO: 8
KDM5D
MEPGCDEFLPPPECPVFEPSWAEFQDPLGYIAKIRPIAEKSGICKIRPPADWQPPFAVEVD
NFRFTPRVQRLNELEAQTRVKLNYLDQIAKFWEIQGSSLKIPNVERKILDLYSLSKIVIEE
GGYEAICKDRRWARVAQRLHYPPGKNIGSLLRSHYERIIYPYEMFQSGANHVQCNTHPF
DNEVKDKEYKPHSIPLRQSVQPSLFSSYSRRAKRLQPDPEPTEEDIEKHPELKKLQIYGPG
PKMMGLGLMAKDKDKTVHKKVTCPPTVTVKDEQSGGGNVSSTLLKQHLSLEPCTKTT
MQLRKNHSSAQFIDSYICQVCSRGDEDDKLLFCDGCDDNYHIFCLLPPLPEIPRGIWRCP
KCILAECKQPPEAFGFEQATQEYSLQSFGEMADSFKSDYFNMPVHMVPTELVEKEFWRL
VSSIEEDVTVEYGADIHSKEFGSGFPVSNSKQNLSPEEKRQSLTVLTRLISSFWAQAVLPP
WPPKVLGLQEYATSGWNLVMPVLDQSVLCHINADISGMKVPWLYVGMVFSAFCWHI
EDHWSYSINYLHWGEPKTWYGVPSLAAEHLEEVMKMLTPELFDSQPDLLHQLVTLMNP
NTLMSHGVPVVRTNQCAGEFVITFPRAYHSGFNQGYNFAEAVNFCTADWLPAGRQCIE
HYRRLRRYCVFHEELICKMAAFPETLDLNLAVAVHKEMFIMVQEERRLRKALLEKGV
TEAEREAFELLPDDERQCIKCKTTCFLSALACYDCPDGLVCLSHINDLCKCSSSRQYLRY
RYTLDELPTMLHKLKIRAESFDTWANKVRVALEVEDGRKRSFEELRALESEARERRFPN
SELLQRLKNCLSEVEACIAQVLGLVSGQVARMDTPQLTLTELRVLLEQMGSLPCAMHQI
GDVKDVLEQVEAYQAEAREALATLPSSPGLLRSLLERGQQLGVEVPEAHQLQQQVEQA
QWLDEVKQALAPSAHRGSLVIMQGLLVMGAKIASSPSVDKARAELQELLTIAERWEEK
AHFCLEARQKHPPATLEAIIRETENIPVHLPNIQALKEALTKAQAWIADVDEIQNGDHYP
CLDDLEGLVAVGRDLPVGLEELRQLELQVLTAHSWREKASKTFLKKNSCYTLLEVLCPC
ADAGSDSTKRSRWMEKALGLYQCDTELLGLSAQDLRDPGSVIVAFKEGEQKEKEGILQ
LRRTNSAKPSPLAPSLMASSPTSICVCGQVPAGVGVLQCDLCQDWFHGQCVSVPHLLTS
PKPSLTSSPLLAWWEWDTKFLCPLCMRSRRPRLETILALLVALQRLPVRLPEGEALQCLT
ERAIGWQDRARKALASEDVTALLRQLAERQQLQAKPRPEEASVYTSATACDPIREGSG
NNISKVQGLEENGDSVTSPENMAPGKGSDLELLSSLLPQLTGPVLELPEAIRAPLEELMM
EGDLLEVTLDENHSIWQLLQAGQPPDLDRIRTLLELEKFEHQGSRTRSRALERRRRRQKV
DQGRNVENLVQQELQSKRARSSGIMSQVGREEEHYQEKADRENMFLTPSTDHSPFLKG
NQNSLQHKDSGSSAACPSLMPLLQLSYSDEQQL

What is claimed is:

1. A method for treating cancer, the method comprising: administering a therapeutically effective amount of an inhibitor of a histone lysine demethylase (KDM) to a subject in need thereof, wherein the histone lysine demethylase is a KDM4 or KDM5 family member, and wherein:
(i) the subject has at least one mutation in an epigenetic modifier selected from the group consisting of EZH2, KMT2D, CREBPP, and EP300;
(ii) the subject has over-expression of at least one Ikaros family member;
(iii) the subject has over-expression of KDM4A and/or KDM4C; and/or
(iv) the subject has at least one mutation in canonical Wnt signaling.

2. The method of claim 1, wherein the subject has at least mutation in an epigenetic modifier selected from the group consisting of EZH2, KMT2D, CREBPP, and EP300.

3. The method of claim 1, wherein subject has over-expression of at least one Ikaros family member.

4. The method of claim 1, wherein the at least one Ikaros family member is IKZF1 and/or IKZF3.

5. (canceled)

6. The method of claim 1, wherein the subject has at least one mutation in canonical Wnt signaling.

7. The method of claim 1, wherein the subject has an activating Wnt-mutation.

8. The method of claim 1, further comprising selecting, prior to onset of treatment, a subject, wherein:
(i) the subject has at least one mutation in an epigenetic modifier selected from the group consisting of EZH2, KMT2D, CREBPP, and EP300;
(ii) the subject has over-expression of at least one Ikaros family member;
(iii) the subject has over-expression of KDM4A and/or KDM4C; and/or
(iv) the subject has at least one mutation in canonical Wnt signaling.

9. The method of claim 1, further comprising assaying, prior to onset of treatment, a biological sample from the subject for presence of the following:
(i) at least one mutation in an epigenetic modifier selected from the group consisting of EZH2, KMT2D, CREBPP, and EP300;
(ii) over-expression of at least one Ikaros family member;
(iii) over-expression of KDM4A and/or KDM4C; and/or
(iv) at least one mutation in canonical Wnt signaling.

10. The method of claim 1, wherein the inhibitor is an inhibitor of a KDM4 family member.

11. The method of claim 10, wherein the KDM4 family member is selected from the group consisting of KDM4A, KDM4B and KDM4C.

12. (canceled)

13. The method of claim 1, wherein the inhibitor is an inhibitor of a KDM5 family member.

14. The method of claim 13, wherein the KDM5 family member is selected from the group consisting of KDM5A and KDM5B.

15. The method of claim 1, wherein the inhibitor is 5-Chloro-2-[(E)-2-[phenyl(pyridin-2-yl)methylidene]hydrazin-1-yl]pyridine (JIB04).

16. The method of claim 1, wherein the inhibitor is administered as a monotherapy.

17. The method of claim 1, further comprising co-administering a cyclin-dependent kinase (Cdk) inhibitor, a Bruton's tyrosine kinase (BTK) inhibitor, or an inhibitor of B-cell receptor (BCR) signaling to the subject.

18. The method of claim 17, wherein the Cdk inhibitor or the BTK inhibitor is administered in an amount that is not effective to treat the cancer when the Cdk inhibitor or the BTK inhibitor is administered alone.

19. The method of claim 18, wherein the Cdk inhibitor is an inhibitor of Cdk7.

20. (canceled)

21. The method of claim 1, wherein the cancer results from increased activation of canonical WNT signaling.

22. The method of claim 1, wherein the cancer is selected from the group consisting of diffuse large B-cell lymphoma (DLBCL), colorectal cancer, acute myeloid leukemia (AML), thymoma, clear cell renal carcinoma, thyroid cancer, glioblastoma (glioblastoma multiforme, GBM), mesothelioma, ovarian cancer, and testicular cancer (Germ Cell Tumors).

23. (canceled)

24. The method of claim 1, further comprising co-administering a second anti-cancer therapy to the subject.


SEQUENCE LISTING (8 sequences)

SEQ ID NO: 1 - protein, 1064 aa, Homo sapiens
MASESETLNPSARIMTFYPTMEEFRNFSRYIAYIESQGAHRAGLAKVVPPKEWKPRASYDDIDDLVIPAPIQQLVTGQSGLFTQYNIQKKAMTVREFRKIANSDKYCTPRYSEFEELERKYWKNLTFNPPIYGADVNGTLYEKHVDEWNIGRLRTILDLVEKESGITIEGVNTPYLYFGMWKTSFAWHTEDMDLYSINYLHFGEPKSWYSVPPEHGKRLERLAKGFFPGSAQSCEAFLRHKMTLISPLMLKKYGIPFDKVTQEAGEFMITFPYGYHAGFNHGFNCAESTNFATRRWIEYGKQAVLCSCRKDMVKISMDVFVRKFQPERYKLWKAGKDNTVIDHTLPTPEAAEFLKESELPPRAGNEEECPEEDMEGVEDGEEGDLKTSLAKHRIGTKRHRVCLEIPQEVSQSELFPKEDLSSEQYEMTECPAALAPVRPTHSSVRQVEDGLTFPDYSDSTEVKFEELKNVKLEEEDEEEEQAAAALDLSVNPASVGGRLVFSGSKKKSSSSLGSGSSRDSISSDSETSEPLSCRAQGQTGVLTVHSYAKGDGRVTVGEPCTRKKGSAARSFSERELAEVADEYMFSLEENKKSKGRRQPLSKLPRHHPLVLQECVSDDETSEQLTPEEEAEETEAWAKPLSQLWQNRPPNFEAEKEFNETMAQQAPHCAVCMIFQTYHQVEFGGFNQNCGNASDLAPQKQRTKPLIPEMCFTSTGCSTDINLSTPYLEEDGTSILVSCKKCSVRVHASCYGVPPAKASEDWMCSRCSANALEEDCCLCSLRGGALQRANDDRWVHVSCAVAILEARFVNIAERSPVDVSKIPLPRFKLKCIFCKKRRKRTAGCCVQCSHGRCPTAFHVSCAQAAGVMMQPDDWPFVVFITCFRHKIPNLERAKGALQSITAGQKVISKHKNGRFYQCEVVRLTTETFYEVNFDDGSFSDNLYPEDIVSQDCLQFGPPAEGEVVQVRWTDGQVYGAKFVASHPIQMYQVEFEDGSQLVVKRDDVYTLDEELPKRVKSRLSVASDMRFNEIFTEKEVKQEKKRQRVINSRYREDYIEPALYRAIME

SEQ ID NO: 2 - protein, 1096 aa, Homo sapiens
MGSEDHGAQNPSCKIMTFRPTMEEFKDFNKYVAYIESQGAHRAGLAKIIPPKEWKPRQTYDDIDDVVIPAPIQQVVTGQSGLFTQYNIQKKAMTVGEYRRLANSEKYCTPRHQDFDDLERKYWKNLTFVSPIYGADISGSLYDDDVAQWNIGSLRTILDMVERECGTIIEGVNTPYLYFGMWKTTFAWHTEDMDLYSINYLHFGEPKSWYAIPPEHGKRLERLAIGFFPGSSQGCDAFLRHKMTLISPIILKKYGIPFSRITQEAGEFMITFPYGYHAGFNHGFNCAESTNFATLRWIDYGKVATQCTCRKDMVKISMDVFVRILQPERYELWKQGKDLTVLDHTRPTALTSPELSSWSASRASLKAKLLRRSHRKRSQPKKPKPEDPKFPGEGTAGAALLEEAGGSVKEEAGPEVDPEEEEEEPQPLPHGREAEGAEEDGRGKLRPTKAKSERKKKSFGLLPPQLPPPPAHFPSEEALWLPSPLEPPVLGPGPAAMEESPLPAPLNVVPPEVPSEELEAKPRPIIPMLYVVPRPGKAAFNQEHVSCQQAFEHFAQKGPTWKEPVSPMELTGPEDGAASSGAGRMETKARAGEGQAPSTFSKLKMEIKKSRRHPLGRPPTRSPLSVVKQEASSDEEASPFSGEEDVSDPDALRPLLSLQWKNRAASFQAERKFNAAAARTEPYCAICTLFYPYCQALQTEKEAPIASLGEGCPATLPSKSRQKTRPLIPEMCFTSGGENTEPLPANSYIGDDGTSPLIACGKCCLQVHASCYGIRPELVNEGWTCSRCAAHAWTAECCLCNLRGGALQMTTDRRWIHVICAIAVPEARFLNVIERHPVDISAIPEQRWKLKCVYCRKRMKKVSGACIQCSYEHCSTSFHVTCAHAAGVLMEPDDWPYVVSITCLKHKSGGHAVQLLRAVSLGQVVITKNRNGLYYRCRVIGAASQTCYEVNFDDGSYSDNLYPESITSRDCVQLGPPSEGELVELRWTDGNLYKAKFISSVTSHIYQVEFEDGSQLTVKRGDIFTLEEELPKRVRSRLSLSTGAPQEPAFSGEEAKAAKRPRVGTPLATEDSGRSQDYVAFVESLLQVQGRPGAPF

SEQ ID NO: 3 - protein, 813 aa, Homo sapiens
MEVAEVESPLNPSCKIMTFRPSMEEFREFNKYLAYMESKGAHRAGLAKVIPPKEWKPRQCYDDIDNLLIPAPIQQMVTGQSGLFTQYNIQKKAMTVKEFRQLANSGKYCTPRYLDYEDLERKYWKNLTFVAPIYGADINGSIYDEGVDEWNIARLNTVLDVVEEECGISIEGVNTPYLYFGMWKTTFAWHTEDMDLYSINYLHFGEPKSWYAIPPEHGKRLERLAQGFFPSSSQGCDAFLRHKMTLISPSVLKKYGIPFDKITQEAGEFMITFPYGYHAGFNHGFNCAESTNFATVRWIDYGKVAKLCTCRKDMVKISMDIFVRKFQPDRYQLWKQGKDIYTIDHTKPTPASTPEVKAWLQRRRKVRKASRSFQCARSTSKRPKADEEEEVSDEVDGAEVPNPDSVTDDLKVSEKSEAAVKLRNTEASSEEESSASRMQVEQNLSDHIKLSGNSCLSTSVTEDIKTEDDKAYAYRSVPSISSEADDSIPLSSGYEKPEKSDPSELSWPKSPESCSSVAESNGVLTEGEESDVESHGNGLEPGEIPAVPSGERNSFKVPSIAEGENKTSKSWRHPLSRPPARSPMTLVKQQAPSDEELPEVLSIEEEVEETESWAKPLIHLWQTKSPNFAAEQEYNATVARMKPHCAICTLLMPYHKPDSSNEENDARWETKLDEVVTSEGKTKPLIPEMCFIYSEENIEYSPPNAFLEEDGTSLLISCAKCCVRVHASCYGIPSHEICDGWLCARCKRNAWTAECCLCNLRGGALKQTKNNKWAHVMCAVAVPEVRFTNVPERTQIDVGRIPLQRLKLGRLGI

SEQ ID NO: 4 - protein, 523 aa, Homo sapiens
METMKSKANCAQNPNCNIMIFHPTKEEFNDFDKYIAYMESQGAHRAGLAKIIPPKEWKARETYDNISEILIATPLQQVASGRAGVFTQYHKKKKAMTVGEYRHLANSKKYQTPPHQNFEDLERKYWKNRIYNSPIYGADISGSLFDENTKQWNLGHLGTIQDLLEKECGVVIEGVNTPYLYFGMWKTTFAWHTEDMDLYSINYLHLGEPKTWYVVPPEHGQRLERLARELFPGSSRGCGAFLRHKVALISPTVLKENGIPFNRITQEAGEFMVTFPYGYHAGFNHGFNCAEAINFATPRWIDYGKMASQCSCGEARVTFSMDAFVRILQPERYDLWKRGQDRAVVDHMEPRVPASQELSTQKEVQLPRRAALGLRQLPSHWARHSPWPMAARSGTRCHTLVCSSLPRRSAVSGTATQPRAAAVHSSKKPSSTPSSTPGPSAQIIHPSNGRRGRGRPPQKLRAQELTLQTPAKRPLLAGTTCTASGPEPEPLPEDGALMDKPVPLSPGLQHPVKASGCSWAPVP

SEQ ID NO: 5 - protein, 1690 aa, Homo sapiens
MAGVGPGGYAAEFVPPPECPVFEPSWEEFTDPLSFIGRIRPLAEKTGICKIRPPKDWQPPFACEVKSFRFTPRVQRLNELEAMTRVRLDFLDQLAKFWELQGSTLKIPVVERKILDLYALSKIVASKGGFEMVTKEKKWSKVGSRLGYLPGKGTGSLLKSHYERILYPYELFQSGVSLMGVQMPNLDLKEKVEPEVLSTDTQTSPEPGTRMNILPKRTRRVKTQSESGDVSRNTELKKLQIFGAGPKVVGLAMGTKDKEDEVTRRRKVTNRSDAFNMQMRQRKGTLSVNFVDLYVCMFCGRGNNEDKLLLCDGCDDSYHTFCLIPPLPDVPKGDWRCPKCVAEECSKPREAFGFEQAVREYTLQSFGEMADNFKSDYFNMPVHMVPTELVEKEFWRLVSSIEEDVIVEYGADISSKDFGSGFPVKDGRRKILPEEEEYALSGWNLNNMPVLEQSVLAHINVDISGMKVPWLYVGMCFSSFCWHIEDHWSYSINYLHWGEPKTWYGVPSHAAEQLEEVMRELAPELFESQPDLLHQLVTIMNPNVLMEHGVPVYRTNQCAGEFVVTFPRAYHSGFNQGYNFAEAVNFCTADWLPIGRQCVNHYRRLRRHCVFSHEELIFKMAADPECLDVGLAAMVCKELTLMTEEETRLRESVVQMGVLMSEEEVFELVPDDERQCSACRTTCFLSALTCSCNPERLVCLYHPTDLCPCPMQKKCLRYRYPLEDLPSLLYGVKVRAQSYDTWVSRVTEALSANFNHKKDLIELRVMLEDAEDRKYPENDLFRKLRDAVKEAETCASVAQLLLSKKQKHRQSPDSGRTRTKLTVEELKAFVQQLFSLPCVISQARQVKNLLDDVEEFHERAQEAMMDETPDSSKLQMLIDMGSSLYVELPELPRLKQELQQARWLDEVRLTLSDPQQVTLDVMKKLIDSGVGLAPHHAVEKAMAELQELLTVSERWEEKAKVCLQARPRHSVASLESIVNEAKNIPAFLPNVLSLKEALQKAREWTAKVEAIQSGSNYAYLEQLESLSAKGRPIPVRLEALPQVESQVAAARAWRERTGRTFLKKNSSHTLLQVLSPRTDIGVYGSGKNRRKKVKELIEKEKEKDLDLEPLSDLEEGLEETRDTAMVVAVFKEREQKEIEAMHSLRAANLAKMTMVDRIEEVKFCICRKTASGFMLQCELCKDWFHNSCVPLPKSSSQKKGSSWQAKEVKFLCPLCMRSRRPRLETILSLLVSLQKLPVRLPEGEALQCLTERAMSWQDRARQALATDELSSALAKLSVLSQRMVEQAAREKTEKIISAELQKAAANPDLQGHLPSFQQSAFNRVVSSVSSSPRQTMDYDDEETDSDEDIRETYGYDMKDTASVKSSSSLEPNLFCDEEIPIKSEEVVTHMWTAPSFCAEHAYSSASKSCSQGSSTPRKQPRKSPLVPRSLEPPVLELSPGAKAQLEELMMVGDLLEVSLDETQHIWRILQATHPPSEDRFLHIMEDDSMEEKPLKVKGKDSSEKKRKRKLEKVEQLFGEGKQKSKELKKMDKPRKKKLKLGADKSKELNKLAKKLAKEEERKKKKEKAAAAKVELVKESTEKKREKKVLDIPSKYDWSGAEESDDENAVCAAQNCQRPCKDKVDWVQCDGGCDEWFHQVCVGVSPEMAENEDYICINCAKKQGPVSPGPAPPPSFIMSYKLPMEDLKETS

SEQ ID NO: 6 - protein, 1580 aa, Homo sapiens
MEAATTLHPGPRPALPLGGPGPLGEFLPPPECPVFEPSWEEFADPFAFIHKIRPIAEQTGICKVRPPPDWQPPFACDVDKLHFTPRIQRLNELEAQTRVKLNFLDQIAKYWELQGSTLKIPHVERKILDLFQLNKLVAEEGGFAVVCKDRKWTKIATKMGFAPGKAVGSHIRGHYERILNPYNLFLSGDSLRCLQKPNLTTDTKDKEYKPHDIPQRQSVQPSETCPPARRAKRMRAERQSLAVLPRLECSGAILAHCNLRLLDSSNSSASASQAMNIKIEPEETTEARTHNLRRRMGCPTPKCENEKEMKSSIKQEPIERKDYIVENEKEKPKSRSKKATNAVDLYVCLLCGSGNDEDRLLLCDGCDDSYHTFCLIPPLHDVPKGDWRCPKCLAQECSKPQEAFGFEQAARDYTLRTFGEMADAFKSDYFNMPVHMVPTELVEKEFWRLVSTIEEDVTVEYGADIASKEFGSGFPVRDGKIKLSPEEEEYLDSGWNLNNMPVMEQSVLAHITADICGMKLPWLYVGMCFSSFCWHIEDHWSYSINYLHWGEPKTWYGVPGYAAEQLENVMKKLAPELFVSQPDLLHQLVTIMNPNTLMTHEVPVYRTNQCAGEFVITFPRAYHSGFNQGFNFAEAVNFCTVDWLPLGRQCVEHYRLLHRYCVFSHDEMICKMASKADVLDVVVASTVQKDMAIMIEDEKALRETVRKLGVIDSERMDFELLPDDERQCVKCKTTCFMSAISCSCKPGLLVCLHHVKELCSCPPYKYKLRYRYTLDDLYPMMNALKLRAESYNEWALNVNEALEAKINKKKSLVSFKALIEESEMKKFPDNDLLRHLRLVTQDAEKCASVAQQLLNGKRQTRYRSGGGKSQNQLTVNELRQFVTQLYALPCVLSQTPLLKDLLNRVEDFQQHSQKLLSEETPSAAELQDLLDVSFEFDVELPQLAEMRIRLEQARWLEEVQQACLDPSSLTLDDMRRLIDLGVGLAPYSAVEKAMARLQELLTVSEHWDDKAKSLLKARPRHSLNSLATAVKEIEEIPAYLPNGAALKDSVQRARDWLQDVEGLQAGGRVPVLDTLIELVTRGRSIPVHLNSLPRLETLVAEVQAWKECAVNTFLTENSPYSLLEVLCPRCDIGLLGLKRKQRKLKEPLPNGKKKSTKLESLSDLERALTESKETASAMATLGEARLREMEALQSLRLANEGKLLSPLQDVDIKICLCQKAPAAPMIQCELCRDAFHTSCVAVPSISQGLRIWLCPHCRRSEKPPLEKILPLLASLQRIRVRLPEGDALRYMIERTVNWQHRAQQLLSSGNLKFVQDRVGSGLLYSRWQASAGQVSDTNKVSQPPGTTSFSLPDDWDNRTSYLHSPFSTGRSCIPLHGVSPEVNELLMEAQLLQVSLPEIQELYQTLLAKPSPAQQTDRSSPVRPSSEKNDCCRGKRDGINSLERKLKRRLEREGLSSERWERVKKMRTPKKKKIKLSHPKDMNNFKLERERSYELVRSAETHSLPSDTSYSEQEDSEDEDAICPAVSCLQPEGDEVDWVQCDGSCNQWFHQVCVGVSPEMAEKEDYICVRCTVKDAPSRK

SEQ ID NO: 7 - protein, 1379 aa, Homo sapiens
MEPGSDDFLPPPECPVFEPSWAEFRDPLGYIAKIRPIAEKSGICKIRPPAIVVEEGGYEAICKDRRWARVAQRLNYPPGKNIGSLLRSHYERIVYPYEMYQSGANLVQCNTRPFDNEEKDKEYKPHSIPLRQSVQPSKFNSYGRRAKRLQPDPEPTEEDIEKNPELKKLQIYGAGPKMMGLGLMAKDKTLRKKDKEGPECPPTVVVKEELGGDVKVESTSPKTFLESKEELSHSPEPCTKMTMRLRRNHSNAQFIESYVCRMCSRGDEDDKLLLCDGCDDNYHIFCLLPPLPEIPKGVWRCPKCVMAECKRPPEAFGFEQATREYTLQSFGEMADSFKADYFNMPVHMVPTELVEKEFWRLVNSIEEDVTVEYGADIHSKEFGSGFPVSDSKRHLTPEEEEYATSGWNLNVMPVLEQSVLCHINADISGMKVPWLYVGMVFSAFCWHIEDHWSYSINYLHWGEPKTWYGVPSLAAEHLEEVMKKLTPELFDSQPDLLHQLVTLMNPNTLMSHGVPVVRTNQCAGEFVITFPRAYHSGFNQGYNFAEAVNFCTADWLPAGRQCIEHYRRLRRYCVFSHEELICKMAACPEKLDLNLAAAVHKEMFIMVQEERRLRKALLEKGITEAEREAFELLPDDERQCIKCKTTCFLSALACYDCPDGLVCLSHINDLCKCSSSRQYLRYRYTLDELPAMLHKLKVRAESFDTWANKVRVALEVEDGRKRSLEELRALESEARERRFPNSELLQQLKNCLSEAEACVSRALGLVSGQEAGPHRVAGLQMTLTELRAFLDQMNNLPCAMHQIGDVKGVLEQVEAYQAEAREALASLPSSPGLLQSLLERGRQLGVEVPEAQQLQRQVEQARWLDEVKRTLAPSARRGTLAVMRGLLVAGASVAPSPAVDKAQAELQELLTIAERWEEKAHLCLEARQKHPPATLEAIIREAENIPVHLPNIQALKEALAKARAWIADVDEIQNGDHYPCLDDLEGLVAVGRDLPVGLEELRQLELQVLTAHSWREKASKTFLKKNSCYTLLEVLCPCADAGSDSTKRSRWMEKELGLYKSDTELLGLSAQDLRDPGSVIVAFKEGEQKEKEGILQLRRTNSAKPSPLASSSTASSTTSICVCGQVLAGAGALQCDLCQDWFHGRCVSVPRLLSSPRPNPTSSPLLAWWEWDTKFLCPLCMRSRRPRLETILALLVALQRLPVRLPEGEALQCLTERAISWQGRARQALASEDVTALLGRLAELRQRLQAEPRPEEPPNYPAAPASDPLREGSGKDMPKVQGLLENGDSVTSPEKVAPEEGSDLELLSSLLPQLTGPVLELPEATRAPLEELMMEGDLLEVTLDENHSIPESLDFCILTPRYCSDLSSWGPAPGVFPPW

SEQ ID NO: 8 - protein, 1570 aa, Homo sapiens
MEPGCDEFLPPPECPVFEPSWAEFQDPLGYIAKIRPIAEKSGICKIRPPADWQPPFAVEVDNFRFTPRVQRLNELEAQTRVKLNYLDQIAKFWEIQGSSLKIPNVERKILDLYSLSKIVIEEGGYEAICKDRRWARVAQRLHYPPGKNIGSLLRSHYERIIYPYEMFQSGANHVQCNTHPFDNEVKDKEYKPHSIPLRQSVQPSKFSSYSRRAKRLQPDPEPTEEDIEKHPELKKLQIYGPGPKMMGLGLMAKDKDKTVHKKVTCPPTVTVKDEQSGGGNVSSTLLKQHLSLEPCTKTTMQLRKNHSSAQFIDSYICQVCSRGDEDDKLLFCDGCDDNYHIFCLLPPLPEIPRGIWRCPKCILAECKQPPEAFGFEQATQEYSLQSFGEMADSFKSDYFNMPVHMVPTELVEKEFWRLVSSIEEDVTVEYGADIHSKEFGSGFPVSNSKQNLSPEEKRQSLTVLTRLISSFWAQAVLPPWPPKVLGLQEYATSGWNLNVMPVLDQSVLCHINADISGMKVPWLYVGMVFSAFCWHIEDHWSYSINYLHWGEPKTWYGVPSLAAEHLEEVMKMLTPELFDSQPDLLHQLVTLMNPNTLMSHGVPVVRTNQCAGEFVITFPRAYHSGFNQGYNFAEAVNFCTADWLPAGRQCIEHYRRLRRYCVFSHEELICKMAAFPETLDLNLAVAVHKEMFIMVQEERRLRKALLEKGVTEAEREAFELLPDDERQCIKCKTTCFLSALACYDCPDGLVCLSHINDLCKCSSSRQYLRYRYTLDELPTMLHKLKIRAESFDTWANKVRVALEVEDGRKRSFEELRALESEARERRFPNSELLQRLKNCLSEVEACIAQVLGLVSGQVARMDTPQLTLTELRVLLEQMGSLPCAMHQIGDVKDVLEQVEAYQAEAREALATLPSSPGLLRSLLERGQQLGVEVPEAHQLQQQVEQAQWLDEVKQALAPSAHRGSLVIMQGLLVMGAKIASSPSVDKARAELQELLTIAERWEEKAHFCLEARQKHPPATLEAIIRETENIPVHLPNIQALKEALTKAQAWIADVDEIQNGDHYPCLDDLEGLVAVGRDLPVGLEELRQLELQVLTAHSWREKASKTFLKKNSCYTLLEVLCPCADAGSDSTKRSRWMEKALGLYQCDTELLGLSAQDLRDPGSVIVAFKEGEQKEKEGILQLRRTNSAKPSPLAPSLMASSPTSICVCGQVPAGVGVLQCDLCQDWFHGQCVSVPHLLTSPKPSLTSSPLLAWWEWDTKFLCPLCMRSRRPRLETILALLVALQRLPVRLPEGEALQCLTERAIGWQDRARKALASEDVTALLRQLAELRQQLQAKPRPEEASVYTSATACDPIREGSGNNISKVQGLLENGDSVTSPENMAPGKGSDLELLSSLLPQLTGPVLELPEAIRAPLEELMMEGDLLEVTLDENHSIWQLLQAGQPPDLDRIRTLLELEKFEHQGSRTRSRALERRRRRQKVDQGRNVENLVQQELQSKRARSSGIMSQVGREEEHYQEKADRENMFLTPSTDHSPFLKGNQNSLQHKDSGSSAACPSLMPLLQLSYSDEQQL